SEQUENCE LISTING (65 sequences)

SEQ ID NO: 1 - protein, 453 aa, Homo sapiens [sequence omitted]

SEQ ID NO: 2 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 3 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 4 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 5 - protein, 764 aa, Homo sapiens
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 6 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 7 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 8 - protein, 137 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 9 - protein, 5 aa, Artificial Sequence
  Description of Artificial Sequence Synthetic peptide
GGGGS

SEQ ID NO: 10 - protein, 10 aa, Artificial Sequence
  Description of Artificial Sequence Synthetic peptide
GGGGSGGGGS

SEQ ID NO: 11 - protein, 15 aa, Artificial Sequence
  Description of Artificial Sequence Synthetic peptide
GGGGSGGGGSGGGGS

SEQ ID NO: 12 - protein, 20 aa, Artificial Sequence
  Description of Artificial Sequence Synthetic peptide
GGGGSGGGGSGGGGSGGGGS

SEQ ID NO: 13 - protein, 25 aa, Artificial Sequence
  Description of Artificial Sequence Synthetic peptide
GGGGSGGGGSGGGGSGGGGSGGGGS

SEQ ID NO: 14 - protein, 125 aa, Artificial Sequence
  Description of Artificial Sequence Synthetic polypeptide
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSS

SEQ ID NO: 15 - protein, 109 aa, Artificial Sequence
  Description of Artificial Sequence Synthetic polypeptide
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 16 - protein, 120 aa, Artificial Sequence [sequence omitted]

SEQ ID NO: 17 - protein, 106 aa, Artificial Sequence [sequence omitted]

SEQ ID NO: 18 - protein, 125 aa, Artificial Sequence
  Description of Artificial Sequence Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSS

SEQ ID NO: 19 - protein, 109 aa, Artificial Sequence
  Description of Artificial Sequence Synthetic polypeptide
QTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVL

SEQ ID NO: 20 - protein, 125 aa, Artificial Sequence; identical to SEQ ID NO: 18
  Description of Artificial Sequence Synthetic polypeptide

SEQ ID NO: 21 - protein, 109 aa, Artificial Sequence
  Description of Artificial Sequence Synthetic polypeptide
QTVVTQEPSLSVSPGGTVTLTCGSSTGAVTTSNYANWVQQTPGQAPRGLIGGTDKRAPGVPDRFSGSLLGDKAALTITGAQAEDEADYYCALWYSDLWVFGGGTKLTVL

SEQ ID NO: 22 - protein, 119 aa, Artificial Sequence
  Description of Artificial Sequence Synthetic polypeptide
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSS

SEQ ID NO: 23 - protein, 112 aa, Artificial Sequence
  Description of Artificial Sequence Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIK

SEQ ID NO: 24 - protein, 119 aa, Artificial Sequence
  Description of Artificial Sequence Synthetic polypeptide
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGRYFYDVWGQGTLVTVSS

SEQ ID NO: 25 - protein, 112 aa, Artificial Sequence
  Description of Artificial Sequence Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYFRRTFGGGTKVEIK

SEQ ID NO: 26 - protein, 119 aa, Artificial Sequence
  Description of Artificial Sequence Synthetic polypeptide
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGQYFYDVWGQGTLVTVSS

SEQ ID NO: 27 - protein, 112 aa, Artificial Sequence
  Description of Artificial Sequence Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCTQSYFRRTFGGGTKVEIK

SEQ ID NO: 28 - protein, 122 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSS

SEQ ID NO: 29 - protein, 112 aa, Artificial Sequence
  Description of Artificial Sequence Synthetic polypeptide
DIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIK

SEQ ID NO: 30 - protein, 585 aa, Homo sapiens
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 31 - protein, 241 aa, Artificial Sequence
  Description of Artificial Sequence Synthetic polypeptide
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIK

SEQ ID NO: 32 - protein, 393 aa, Artificial Sequence
  Description of Artificial Sequence Synthetic polypeptide
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 33 - protein, 393 aa, Artificial Sequence
  Description of Artificial Sequence Synthetic polypeptide
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 34 - protein, 993 aa, Artificial Sequence
  Description of Artificial Sequence Synthetic polypeptide
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 35 - protein, 249 aa, Artificial Sequence
  Description of Artificial Sequence Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVL

SEQ ID NO: 36 - protein, 401 aa, Artificial Sequence
  Description of Artificial Sequence Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 37 - protein, 401 aa, Artificial Sequence
  Description of Artificial Sequence Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 38 - protein, 1001 aa, Artificial Sequence
  Description of Artificial Sequence Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 39 - protein, 249 aa, Artificial Sequence
  Description of Artificial Sequence Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSDLWVFGGGTKLTVL

SEQ ID NO: 40 - protein, 401 aa, Artificial Sequence
  Description of Artificial Sequence Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSDLWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 41 - protein, 401 aa, Artificial Sequence
  Description of Artificial Sequence Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSDLWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 42 - protein, 1001 aa, Artificial Sequence
  Description of Artificial Sequence Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSDLWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 43 - protein, 246 aa, Artificial Sequence
  Description of Artificial Sequence Synthetic polypeptide
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIK

SEQ ID NO: 44 - protein, 398 aa, Artificial Sequence
  Description of Artificial Sequence Synthetic polypeptide
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 45 - protein, 398 aa, Artificial Sequence
  Description of Artificial Sequence Synthetic polypeptide
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 46 - protein, 846 aa, Artificial Sequence
  Description of Artificial Sequence Synthetic polypeptide
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 47 - protein, 246 aa, Artificial Sequence
  Description of Artificial Sequence Synthetic polypeptide
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGRYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYFRRTFGGGTKVEIK

SEQ ID NO: 48 - protein, 398 aa, Artificial Sequence
  Description of Artificial Sequence Synthetic polypeptide
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGRYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYFRRTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 49 - protein, 398 aa, Artificial Sequence
  Description of Artificial Sequence Synthetic polypeptide
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGRYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYFRRTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 50 - protein, 998 aa, Artificial Sequence
  Description of Artificial Sequence Synthetic polypeptide
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGRYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYFRRTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 51 - protein, 246 aa, Artificial Sequence
  Description of Artificial Sequence Synthetic polypeptide
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGQYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCTQSYFRRTFGGGTKVEIK

SEQ ID NO: 52 - protein, 398 aa, Artificial Sequence
  Description of Artificial Sequence Synthetic polypeptide
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGQYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCTQSYFRRTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 53 - protein, 398 aa, Artificial Sequence
  Description of Artificial Sequence Synthetic polypeptide
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGQYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCTQSYFRRTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 54 - protein, 998 aa, Artificial Sequence
  Description of Artificial Sequence Synthetic polypeptide
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGQYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCTQSYFRRTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 55 - protein, 10 aa, Artificial Sequence
  Description of Artificial Sequence Synthetic peptide
GYSFTSYWIG

SEQ ID NO: 56 - protein, 17 aa, Artificial Sequence
  Description of Artificial Sequence Synthetic peptide
IIYPGDSDTRYSPSFQG

SEQ ID NO: 57 - protein, 13 aa, Artificial Sequence
  Description of Artificial Sequence Synthetic peptide
HPSYGSGSPNFDY

SEQ ID NO: 58 - protein, 16 aa, Artificial Sequence
  Description of Artificial Sequence Synthetic peptide
RSSQSLVYSDGNTYLS

SEQ ID NO: 59 - protein, 7 aa, Artificial Sequence
  Description of Artificial Sequence Synthetic peptide
KISNRFS

SEQ ID NO: 60 - protein, 9 aa, Artificial Sequence
  Description of Artificial Sequence Synthetic peptide
VQATQFPLT

SEQ ID NO: 61 - protein, 575 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 62 - protein, 219 aa, Artificial Sequence
  Description of Artificial Sequence Synthetic polypeptide
DIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 452 aa, Artificial Sequence
  Description of Artificial Sequence Synthetic polypeptide
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 64 - protein, 214 aa, Artificial Sequence
  Description of Artificial Sequence Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 448 aa, Artificial Sequence
  Description of Artificial Sequence Synthetic polypeptide
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLG

(19) United States
(12) Patent Application Publication
(10) Pub. No.: US 2024/0002526 A1
(43) Pub. Date: Jan. 4, 2024

(54) USES OF EFFECTOR CELL ENGAGING MOLECULES WITH MOIETIES OF DIFFERING POTENCIES

(71) Applicant: IGM Biosciences, Inc., Mountain View, CA (US)

(72) Inventors: Daniel S. CHEN, Burlingame, CA (US); Wayne R. GODFREY, Bainbridge Island, WA (US); Maria Genevive Hermoso HERNANDEZ, Millbrae, CA (US); Iris SISON, Livermore, CA (US); Ibrahim QAZI, Columbia, MD (US); Kevin Charles HART, Los Gatos, CA (US); Maya Fay KOTTURI, Belmont, CA (US)

(21) Appl. No.: 18/252,759

(22) PCT Filed: Nov. 17, 2021
(86) PCT No.: PCT/US2021/059721

§ 371 Date: May 12, 2023

Related U.S. Application Data
(60) Provisional application No. 63/114,991, filed on Nov. 17, 2020.
(60) Provisional application No. 63/254,508, filed on Oct. 11, 2021.

(51) Int. Cl. C07K 16/28; A61P 35/00

(52) U.S. Cl. CPC C07K 16/2887; C07K 16/2809; A61P 35/00; A61K 2039/545; C07K 2317/31; C07K 2317/622; C07K 2317/35

(57) ABSTRACT

This disclosure provides methods of treating cancer comprising administering effector cell engaging molecules comprising one or more tumor targeting moieties and one or more effector cell engaging moieties. For example, the effector cell engaging molecules can have a greater cumulative potency for the tumor targeting moieties than the effector cell engaging moieties, the tumor targeting moieties can have greater avidity than the effector cell engaging moieties, and/or can be multimeric. The methods comprise, for example, administering different doses of effector cell engaging molecules based on the subject's symptoms and/or administering chimeric antigen receptor expressing cells.

### CROSS-REFERENCE TO RELATED APPLICATIONS

This application claims the benefit of U.S. Provisional Patent Application Ser. No. 63/114,991, filed Nov. 17, 2020 and 63/254,508, filed Oct. 11, 2021, which are each incorporated herein by reference in their entireties.

### SEQUENCE LISTING

The instant application contains a Sequence Listing which has been submitted electronically in ASCII format and is hereby incorporated by reference in its entirety. The ASCII copy was created on Nov. 16, 2021, is named 033WO1-Sequence-Listing, and is 174,378 bytes in size.

### BACKGROUND

Bispecific antibodies that bridge tumor cells, to effector cells, e.g., T cells, have shown promise in treating various malignancies such as B cell malignancies such as lymphoma. See, e.g., Budde L E, et al. Blood 2018; 132(Suppl. 1):399; Bannerji R, et al. Blood 2019; 134(Suppl. 1):762; and Li J, et. al. Sci Transl Med 2019; 11(508): eaax8861. Most T-cell engaging antibodies in preclinical and clinical evaluations, however, are associated with toxicity (especially cytokine release syndrome (CRS)).

IGM-2323 is a novel bispecific antibody, based on an engineered pentameric IgM framework, with a recombinant J-chain that is fused to an anti-CD3 scFv, and also to human serum albumin to increase serum half-life. See, e.g., U.S. Pat. Nos. 10,787,520 and 10,618,978, which are incorporated herein by reference in their entireties. In preclinical studies, IGM-2323 was shown to bind irreversibly to CD20-expressing cells, including cancer cells expressing very low levels of CD20, and eliminate them through cell-dependent (TDCC) and cell-independent mechanisms (CDC). Importantly, IGM-2323 did not induce significant cytokine release in in vitro or in vivo preclinical studies. See, e.g., Baliga, R, et al., Blood (2019) 134 (Supplement_1): 1574.

There remains a need for cancer therapies that can engage effector cells, e.g., T cells, to tumor cells without over stimulating the effector function to allow for tumor cell killing with minimal toxicity.

### SUMMARY

Provided herein is a method of treating cancer in a subject in need thereof, comprising: (a) administering a dosage of 10 mg to 50 mg of a T cell engaging molecule to the subject; (b) administering a dosage of 75 mg to 600 mg of the T cell engaging molecule to the subject at least 5 days after the administration of step (a), where the T cell engaging molecule is a multimeric binding molecule comprising five bivalent binding units and a modified J-chain, where each binding unit comprises two IgM heavy chains, each comprising a heavy chain variable region (VH) and an IgM constant region and two light chains, each comprising a light chain variable region (VL) and a light chain constant region, where an associated VH and VL specifically bind to CD20 or a subunit thereof, where the VH comprises three immunoglobulin complementarity determining regions: HCDR1, HCDR2, and HCDR3, and the VL comprises three immunoglobulin complementarity determining regions: LCDR1, LCDR2, and LCDR3, where the HCDR1, HCDR2, HCDR3, LCDR1, LCDR2, and LCDR3 comprise, respectively, the amino acid sequence of SEQ ID NO: 55; SEQ ID NO: 56; SEQ ID NO: 57; SEQ ID NO: 58; SEQ ID NO: 59; and SEQ ID NO: 60, where the modified J-chain comprises a J-chain or functional fragment or variant thereof and an scFv molecule that specifically binds to CD3, and where the scFv comprises the amino acid sequence of SEQ ID NO: 31.

In some embodiments, the method further comprises (c) administering the dosage of the T cell engaging molecule administered to the subject in step (b) at least 5 days after the administration of step (b). In some embodiments, the dosage of the T cell engaging molecule in step (b) is 100 mg to 300 mg. In some embodiments, the dosage of the T cell engaging molecule in step (b) is 100 mg. In some embodiments, the dosage of the T cell engaging molecule in step (b) is 300 mg.

Also provided herein is a method of treating cancer in a subject in need thereof, comprising administering a dosage of 75 mg to 600 mg of the T cell engaging molecule to the subject, where the T cell engaging molecule is a multimeric binding molecule comprising five bivalent binding units and a modified J-chain, where each binding unit comprises two IgM heavy chains, each comprising a heavy chain variable region (VH) and an IgM constant region and two light chains, each comprising a light chain variable region (VL) and a light chain constant region, where an associated VH and VL specifically bind to CD20 or a subunit thereof, where the VH comprises three immunoglobulin complementarity determining regions: HCDR1, HCDR2, and HCDR3, and the VL comprises three immunoglobulin complementarity determining regions: LCDR1, LCDR2, and LCDR3, where the HCDR1, HCDR2, HCDR3, LCDR1, LCDR2, and LCDR3 comprise, respectively, the amino acid sequence of SEQ ID NO: 55; SEQ ID NO: 56; SEQ ID NO: 57; SEQ ID NO: 58; SEQ ID NO: 59; and SEQ ID NO: 60, where the modified J-chain comprises a J-chain or functional fragment or variant thereof and an scFv molecule that specifically binds to CD3, and where the scFv comprises the amino acid sequence of SEQ ID NO: 31.

In some embodiments, the dosage is 100 mg to 300 mg. In some embodiments, the dosage is 100 mg. In some embodiments, the subject had received a prior dosage of the T cell engaging molecule, where the prior dosage was less than 100 mg. In some embodiments, the prior dosage was 10-50 mg. In some embodiments, the prior dosage comprised two or three prior dosages of the T cell engaging molecule, where the two or three prior dosages were less than 100 mg.

Also provided herein is a method of treating cancer in a subject in need thereof, comprising: (a) administering to the subject a T cell engaging molecule at a determined dosage; (b) monitoring the subject for an administration-related symptom; and (c) (i) administering the T cell engaging molecule to the subject at the same or a reduced dosage relative to the determined dosage if the subject had the administration-related symptom, and (ii) administering the T cell engaging molecule to the subject at an increased dosage relative to the determined dosage if the subject did not have the administration-related symptom, where the T cell engaging molecule is a multimeric binding molecule comprising five bivalent binding units and a modified J-chain, where each binding unit comprises two IgM heavy chains, each comprising a heavy chain variable region (VH) and an IgM constant region and two light chains, each comprising a light chain variable region (VL) and a light chain constant region, where an associated VH and VL specifically bind to CD20 or a subunit thereof, where the VH comprises three immunoglobulin complementarity determining regions: HCDR1, HCDR2, and HCDR3, and the VL comprises three immunoglobulin complementarity determining regions: LCDR1, LCDR2, and LCDR3, where the HCDR1, HCDR2, HCDR3, LCDR1, LCDR2, and LCDR3 comprise, respectively, the amino acid sequence of SEQ ID NO: 55; SEQ ID NO: 56; SEQ ID NO: 57; SEQ ID NO: 58; SEQ ID NO: 59; and SEQ ID NO: 60, where the modified J-chain comprises a J-chain or functional fragment or variant thereof and an scFv molecule that specifically binds to CD3, where the scFv comprises the amino acid sequence of SEQ ID NO: 31.

In some embodiments, the administration-related symptom comprises hypotension, chills, fever, elevated C reactive protein (CRP) level, fatigue, nausea, vomiting, insomnia, back pain, cytokine release, or a combination thereof. In some embodiments, the administration-related symptom comprises hypotension, chills, fever, elevated C reactive protein (CRP) level, or a combination thereof.

In some embodiments, the method further comprises: (d) repeating steps (b) and (c) one or more times. In some embodiments, the increased dosage of the T cell engaging molecule in step (c)(ii) is 25%-1000% greater than the determined dosage of the T cell engaging molecule. In some embodiments, the increased dosage of the T cell engaging molecule in step (c)(ii) is 30 mg to 300 mg. In some embodiments, the increased dosage of the T cell engaging molecule in step (c)(ii) is 100 mg to 300 mg. In some embodiments, the increased dosage of the T cell engaging molecule in step (c)(ii) is 100 mg or 300 mg.

In some embodiments, step (a), step (c)(i), and/or step (c)(ii) further comprises administering dexamethasone.

In some embodiments, the subject had previously received a different cancer therapy. In some embodiments, the different cancer therapy comprises chemotherapy, radiation therapy, or immunotherapy, where the immunotherapy is different from the T cell engaging molecule. In some embodiments, the immunotherapy is rituximab.

Also provided herein is a method of treating cancer in a subject in need thereof, the method comprising administering a T cell engaging molecule to the subject, where the T cell engaging molecule is a multimeric binding molecule comprising five bivalent binding units and a modified J-chain, where each binding unit comprises two IgM heavy chains, each comprising a heavy chain variable region (VH) and an IgM constant region and two light chains, each comprising a light chain variable region (VL) and a light chain constant region, where an associated VH and VL specifically bind to CD20 or a subunit thereof, where the VH comprises three immunoglobulin complementarity determining regions: HCDR1, HCDR2, and HCDR3, and the VL comprises three immunoglobulin complementarity determining regions: LCDR1, LCDR2, and LCDR3, where the HCDR1, HCDR2, HCDR3, LCDR1, LCDR2, and LCDR3 comprise, respectively, the amino acid sequence of SEQ ID NO: 55; SEQ ID NO: 56; SEQ ID NO: 57; SEQ ID NO: 58; SEQ ID NO: 59; and SEQ ID NO: 60, where the modified J-chain comprises a J-chain or functional fragment or variant thereof and an scFv molecule that specifically binds to CD3, where the scFv comprises the amino acid sequence of SEQ ID NO: 31, and where the subject had previously been administered rituximab.

A method of treating cancer in a subject in need thereof, the method comprising administering rituximab to the subject, and administering a T cell engaging molecule to the subject, where the T cell engaging molecule is a multimeric binding molecule comprising five bivalent binding units and a modified J-chain, where each binding unit comprises two IgM heavy chains, each comprising a heavy chain variable region (VH) and an IgM constant region and two light chains, each comprising a light chain variable region (VL) and a light chain constant region, where an associated VH and VL specifically bind to CD20 or a subunit thereof, where the VH comprises three immunoglobulin complementarity determining regions: HCDR1, HCDR2, and HCDR3, and the VL comprises three immunoglobulin complementarity determining regions: LCDR1, LCDR2, and LCDR3, where the HCDR1, HCDR2, HCDR3, LCDR1, LCDR2, and LCDR3 comprise, respectively, the amino acid sequence of SEQ ID NO: 55; SEQ ID NO: 56; SEQ ID NO: 57; SEQ ID NO: 58; SEQ ID NO: 59; and SEQ ID NO: 60, where the modified J-chain comprises a J-chain or functional fragment or variant thereof and an scFv molecule that specifically binds to CD3, and where the scFv comprises the amino acid sequence of SEQ ID NO: 31.

In some embodiments, the administering of the T cell engaging molecule occurs at least 1 week, at least 2 weeks, at least 1 month, at least 2 months, at least 3 months, at least 6 months, at least 9 months, at least 1 year, or at least 2 years after the rituximab was administered. In some embodiments, the subject has rituximab serum levels of 0 μg/mL to 200 μg/mL. In some embodiments, the subject has rituximab serum levels of 0 μg/mL to 100 μg/mL.

In some embodiments, the rituximab administration comprises administering a dose of 375 mg/m 2 of rituximab. In some embodiments, the rituximab administration comprises administering rituximab once weekly for 4 to 8 weeks.

In some embodiments, the immunotherapy is chimeric antigen receptor (CAR)-expressing T cells. In some embodiments, the method further comprises administering chimeric antigen receptor (CAR)-expressing T cells to the subject.

Also provided herein is a method of treating cancer in a subject in need thereof, the method comprising: (1) administering chimeric antigen receptor (CAR)-expressing T cells to the subject; and (2) administering a T cell engaging molecule to the subject, where the T cell engaging molecule is a multimeric binding molecule comprising five bivalent binding units and a modified J-chain, where each binding unit comprises two IgM heavy chains, each comprising a heavy chain variable region (VH) and an IgM constant region and two light chains, each comprising a light chain variable region (VL) and a light chain constant region, where an associated VH and VL specifically bind to CD20 or a subunit thereof, where the VH comprises three immunoglobulin complementarity determining regions: HCDR1, HCDR2, and HCDR3, and the VL comprises three immunoglobulin complementarity determining regions: LCDR1, LCDR2, and LCDR3, where the HCDR1, HCDR2, HCDR3, LCDR1, LCDR2, and LCDR3 comprise, respectively, the amino acid sequence of SEQ ID NO: 55; SEQ ID NO: 56; SEQ ID NO: 57; SEQ ID NO: 58; SEQ ID NO: 59; and SEQ ID NO: 60, where the modified J-chain comprises a J-chain or functional fragment or variant thereof and an scFv molecule that specifically binds to CD3, and where the scFv comprises the amino acid sequence of SEQ ID NO: 31.

Also provided herein is a method of treating cancer in a subject in need thereof, the method comprising administering a T cell engaging molecule to the subject, where the T cell engaging molecule is a multimeric binding molecule comprising five bivalent binding units and a modified J-chain, where each binding unit comprises two IgM heavy chains, each comprising a heavy chain variable region (VH) and an IgM constant region and two light chains, each comprising a light chain variable region (VL) and a light chain constant region, where an associated VH and VL specifically bind to CD20 or a subunit thereof, where the VH comprises three immunoglobulin complementarity determining regions: HCDR1, HCDR2, and HCDR3, and the VL comprises three immunoglobulin complementarity determining regions: LCDR1, LCDR2, and LCDR3, where the HCDR1, HCDR2, HCDR3, LCDR1, LCDR2, and LCDR3 comprise, respectively, the amino acid sequence of SEQ ID NO: 55; SEQ ID NO: 56; SEQ ID NO: 57; SEQ ID NO: 58; SEQ ID NO: 59; and SEQ ID NO: 60, where the modified J-chain comprises a J-chain or functional fragment or variant thereof and an scFv molecule that specifically binds to CD3, where the scFv comprises the amino acid sequence of SEQ ID NO: 31, and where the subject had previously been administered chimeric antigen receptor (CAR)-expressing T cells.

In some embodiments, the CAR-T cells are lisocabtagene maraleucel, axicabtagene ciloleucel, tisagenlecleucel, or brexucabtagene autoleucel.

In some embodiments, the IgM heavy chain constant regions or multimerizing fragments or variants thereof are human IgM constant regions. In some embodiments, the IgM heavy chain constant regions each comprise the amino acid sequence SEQ ID NO: 1, SEQ ID NO: 2, or a multimerizing fragment or variant thereof. In some embodiments, the heavy chain comprises SEQ ID NO: 61 and the light chain comprises SEQ ID NO: 62.

In some embodiments, the J-chain or functional fragment or variant thereof comprises SEQ ID NO: 7. In some embodiments, the modified J-chain further comprises an albumin. In some embodiments, the albumin comprises human serum albumin. In some embodiments, the J-chain or fragment or variant thereof comprises SEQ ID NO: 34.

In some embodiments, the subject is human. In some embodiments, the cancer is a CD20 positive cancer. In some embodiments, the cancer is a leukemia, lymphoma, or myeloma. In some embodiments, the cancer is non-Hodgkin lymphoma (NEIL). In some embodiments, the NHL is diffuse large B-cell lymphoma (DLBCL), follicular lymphoma (FL), mantle cell lymphoma (MCL), or marginal zone lymphoma (MZL). In some embodiments, the cancer is relapsed or refractory cancer.

Provided herein is a method of treating cancer in a subject in need thereof, comprising: (a) administering to the subject an effector cell engaging molecule at a determined dosage; (b) monitoring the subject for an administration-related symptom; and (c)(i) administering the effector cell engaging molecule to the subject at the same or a reduced dosage relative to the determined dosage if the subject had the administration-related symptom, and (ii) administering the effector cell engaging molecule to the subject at an increased dosage relative to the determined dosage if the subject did not have the administration-related symptom, where the effector cell engaging molecule comprises one or more tumor targeting moieties that bind a tumor antigen and one or more effector cell engaging moieties, and where the cumulative potency of the one or more tumor targeting moieties is greater than the cumulative potency of the one or more effector cell engaging moieties.

Also provided herein is a method of treating cancer in a subject in need thereof, comprising: (a) administering to the subject an effector cell engaging molecule at a determined dosage; (b) monitoring the subject for an administration-related symptom; and (c)(i) administering the effector cell engaging molecule to the subject at the same or a reduced dosage relative to the determined dosage if the subject had the administration-related symptom, and (ii) administering the effector cell engaging molecule to the subject at an increased dosage relative to the determined dosage if the subject did not have the administration-related symptom, where the effector cell engaging molecule comprises one or more tumor targeting moieties that bind a tumor antigen and one or more effector cell engaging moieties, and where the total avidity of the one or more tumor targeting moieties is greater than the total avidity of the one or more effector cell engaging moieties.

Also provided herein is a method of treating cancer in a subject in need thereof, comprising: (a) administering to the subject an effector cell engaging molecule at a determined dosage; (b) monitoring the subject for an administration-related symptom; and (c)(i) administering the effector cell engaging molecule to the subject at the same or a reduced dosage relative to the determined dosage if the subject had the administration-related symptom, and (ii) administering the effector cell engaging molecule to the subject at an increased dosage relative to the determined dosage if the subject did not have the administration-related symptom, where the effector cell engaging molecule comprises tumor targeting moieties that bind a tumor antigen and one or more effector cell engaging moieties, where the effector cell engaging molecule is a multimeric binding molecule comprising two to five bivalent binding units or variants or fragments thereof and a J-chain or functional fragment or variant thereof, and where each binding unit comprises two IgA or IgM heavy chain constant regions or multimerizing fragments or variants thereof, each associated with one of the tumor targeting moieties or a subunit thereof.

In some embodiments, the administration-related symptom comprises chills, fever, elevated C reactive protein (CRP) level, fatigue, nausea, vomiting, insomnia, back pain, cytokine release, or a combination thereof. In some embodiments, the administration-related symptom comprises chills, fever, elevated C reactive protein (CRP) level, or a combination thereof.

In some embodiments, the method further comprises (d) repeating steps (b) and (c) one or more times.

In some embodiments, the determined dosage of the effector cell engaging molecule is 30 to 1000 mg. In some embodiments, the determined dosage of the effector cell engaging molecule is 50 mg, 100 mg, 200 mg, or 500 mg. In some embodiments, the increased dosage of the effector cell engaging molecule in step (c)(ii) is 25%-1000% greater than the determined dosage of the effector cell engaging molecule.

In some embodiments, step (a) further comprises administering dexamethasone. In some embodiments, step (c)(i) further comprises administering a lower dosage of dexamethasone or no dexamethasone. In some embodiments, step (c)(i) further comprises administering the same dosage of dexamethasone. In some embodiments, step (c)(ii) further comprises administering the same or a lower dosage of dexamethasone. In some embodiments, the step (a) and the step (c)(i) or (c)(ii) administrations of the effector cell engaging molecule are 3 days to 2 weeks apart.

In some embodiments, the step (a) and the step (c)(i) or (c)(ii) administrations of the effector cell engaging molecule are 1 week apart.

In some embodiments, the subject had previously received a different cancer therapy. In some embodiments, the different cancer therapy comprises chemotherapy, radiation therapy, or immunotherapy, where the immunotherapy is different from the effector cell engaging molecule. In some embodiments, the immunotherapy is chimeric antigen receptor (CAR)-expressing cells. In some embodiments, the method further comprises administering chimeric antigen receptor (CAR)-expressing cells to the subject.

Also provided herein is a method of treating cancer in a subject in need thereof, the method comprising: (1) administering chimeric antigen receptor (CAR)-expressing cells to the subject; and (2) administering an effector cell engaging molecule to the subject, where the effector cell engaging molecule comprises one or more tumor targeting moieties that bind a tumor antigen and one or more effector cell engaging moieties, and where the cumulative potency of the one or more tumor targeting moieties is greater than the cumulative potency of the one or more effector cell engaging moieties.

Also provided herein is a method of treating cancer in a subject in need thereof, the method comprising: (1) administering chimeric antigen receptor (CAR)-expressing cells to the subject; and (2) administering an effector cell engaging molecule to the subject, where the effector cell engaging molecule comprises one or more tumor targeting moieties that bind a tumor antigen and one or more effector cell engaging moieties, and where the total avidity of the one or more tumor targeting moieties is greater than the total avidity of the one or more effector cell engaging moieties.

Also provided herein is a method of treating cancer in a subject in need thereof, the method comprising: (1) administering chimeric antigen receptor (CAR)-expressing cells to the subject; and (2) administering an effector cell engaging molecule to the subject, where effector cell engaging molecule comprises tumor targeting moieties that bind a tumor antigen and one or more effector cell engaging moieties, where the effector cell engaging molecule is a multimeric binding molecule comprising two to five bivalent binding units or variants or fragments thereof and a J-chain or functional fragment or variant thereof, and where each binding unit comprises two IgA or IgM heavy chain constant regions or multimerizing fragments or variants thereof, each associated with one of the tumor targeting moieties or a subunit thereof.

In some embodiments, the CAR-expressing cells are administered prior to the effector cell engaging molecule. In some embodiments, the CAR-expressing cells are administered at least 2 weeks, at least 1 month, at least 2 months, at least 3 months prior to effector cell engaging molecule.

Also provided herein is a method of treating cancer in a subject in need thereof, the method comprising administering an effector cell engaging molecule to the subject, where the effector cell engaging molecule comprises one or more tumor targeting moieties that bind a tumor antigen and one or more effector cell engaging moieties, where the cumulative potency of the one or more tumor targeting moieties is greater than the cumulative potency of the one or more effector cell engaging moieties, and where the subject had previously been administered chimeric antigen receptor (CAR)-expressing cells.

Also provided herein is a method of treating cancer in a subject in need thereof, the method comprising administering an effector cell engaging molecule to the subject, where effector cell engaging molecule comprises one or more tumor targeting moieties that bind a tumor antigen and one or more effector cell engaging moieties, where the total avidity of the one or more tumor targeting moieties is greater than the total avidity of the one or more effector cell engaging moieties, and where the subject had previously been administered chimeric antigen receptor (CAR)-expressing cells.

Also provided herein is a method of treating cancer in a subject in need thereof, the method comprising administering an effector cell engaging molecule to the subject, where effector cell engaging molecule comprises tumor targeting moieties that bind a tumor antigen and one or more effector cell engaging moieties, where the effector cell engaging molecule is a multimeric binding molecule comprising two to five bivalent binding units or variants or fragments thereof and a J-chain or functional fragment or variant thereof, where each binding unit comprises two IgA or IgM heavy chain constant regions or multimerizing fragments or variants thereof, each associated with one of the tumor targeting moieties or a subunit thereof, and where the subject had previously been administered chimeric antigen receptor (CAR)-expressing cells.

In some embodiments, the administering of the effector cell engaging molecule occurs at least 1 week, at least 2 weeks, at least 1 month, at least 2 months, or at least 3 months after the CAR-expressing cells were administered. In some embodiments, the CAR-expressing cells are below the level of detection prior to administering the effector cell engaging molecule.

In some embodiments, the CAR comprises an external targeting domain, where the external targeting domain binds CD19, BCMA, CD20, CD22, CD23, or ROR1. In some embodiments, the CAR expressing cells are CAR-T cells. In some embodiments, the CAR comprises a CD3 signaling domain, a 4-1BB stimulatory domain, and/or a CD28 stimulatory domain. In some embodiments, the CAR-T cells are lisocabtagene maraleucel, axicabtagene ciloleucel, tisagenlecleucel, brexucabtagene autoleucel, idecabtagene vicleucel, ciltacabtagene autoleucel, or orvacabtagene autoleucel.

In some embodiments, the effector cell engaging moieties stimulate effector cells at a more physiologic level than a reference effector cell engaging molecule comprising one or more reference tumor targeting moieties and one or more reference effector cell engaging moieties, where the cumulative potency of the one or more reference tumor targeting moieties is not greater than the cumulative potency of the one or more reference effector cell engaging moieties.

In some embodiments, the ratio of the total avidity of the one or more tumor targeting moieties and the total avidity of the one or more effector cell engaging moieties (avidity T:E) is greater than a reference avidity ratio, where the reference avidity ratio is the ratio of the avidities of the tumor targeting and effector cell engaging moieties in mosunetuzumab. In some embodiments, the avidity T:E is at least 2-fold, four-fold, five-fold, or six-fold greater than the reference avidity ratio.

In some embodiments, the effector cell engaging molecule comprises two, four, eight, or ten tumor targeting moieties. In some embodiments, the effector cell engaging molecule comprises one or two effector cell engaging moieties. In some embodiments, the ratio of the tumor targeting moieties to the effector cell engaging moieties is 2:1, 4:1, 8:1, or 10:1. In some embodiments, the ratio of tumor targeting moieties to effector cell engaging moieties is 4:1 or

In some embodiments, the effector cell is a T cell or a natural killer (NK) cell. In some embodiments, the effector cell is an NK cell. In some embodiments, the one or more NK cell engaging moieties bind CD16.

In some embodiments, the effector cell is a T cell. In some embodiments, the one or more T cell engaging moieties bind CD3. In some embodiments, the one or more T cell engaging moieties comprise a scFv with VH and VL amino acid sequences SEQ ID NO: 14 and SEQ ID NO: 15, SEQ ID NO: 16 and SEQ ID NO: 17, SEQ ID NO: 18 and SEQ ID NO: 19, SEQ ID NO: 20 and SEQ ID NO: 21, SEQ ID NO: 22 and SEQ ID NO: 23, SEQ ID NO: 24 and SEQ ID NO: 25, or SEQ ID NO: 26 and SEQ ID NO: 27, respectively. In some embodiments, the scFv comprises the amino acid sequence of SEQ ID NO: 31, SEQ ID NO: 35, SEQ ID NO: 39, SEQ ID NO: 43, SEQ ID NO: 47, or SEQ ID NO: 51.

In some embodiments, the one or more effector cell engaging moieties comprise an antibody or antigen-binding fragment thereof. In some embodiments, the one or more effector cell engaging moieties comprise a Fab, a Fab′, a F(ab′)2, a Fd, a Fv, a single-chain Fv (scFv), a disulfide-linked Fv (sdFv), or any combination thereof. In some embodiments, the one or more effector cell engaging moieties comprise a scFv.

In some embodiments, the tumor antigen is CD20, CD38, CD123, TROP-2, mesothelin, glypican-3 (GPC-3), PSMA, globo-H, CSPG-4, or CEA. In some embodiments, the tumor antigen is CD20.

In some embodiments, the one or more tumor targeting moieties comprise an antibody or antigen-binding fragment thereof. In some embodiments, the one or more tumor targeting moieties comprise a Fab, a Fab′, a F(ab′)2, a Fd, a Fv, a single-chain Fv (scFv), a disulfide-linked Fv (sdFv), or any combination thereof.

In some embodiments, the effector cell engaging molecule is a multimeric binding molecule comprising two to five bivalent binding units or variants or fragments thereof and a J-chain or functional fragment or variant thereof, where each binding unit comprises two IgA or IgM heavy chain constant regions or multimerizing fragments or variants thereof, each associated with one of the tumor targeting moieties or a subunit thereof.

In some embodiments, the tumor targeting moieties comprise immunoglobulin antigen binding domains comprising a heavy chain variable region (VH) and a light chain variable region (VL).

In some embodiments, the immunoglobulin antigen-binding domains are human or humanized antigen binding domains.

In some embodiments, the tumor antigen is CD20, and the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 28 and SEQ ID NO: 29, respectively with zero, one, or two single amino acid substitutions in one or more of the HCDRs or LCDRs. In some embodiments, the tumor antigen is CD20, and where the VH and VL comprise amino acid sequences at least 80%, at least 85%, at least 90%, at least 95% or 100% identical to SEQ ID NO: 28 and SEQ ID NO: 29, respectively.

In some embodiments, each binding unit comprises two heavy chains comprising the VH and two light chains comprising the VL.

In some embodiments, the multimeric binding molecule comprises two or four bivalent IgA or IgA-like binding units, where each binding unit comprises two IgA heavy chain constant regions or multimerizing fragments or variants thereof, each comprising an IgA Cα3 domain and an IgA tailpiece domain. In some embodiments, the multimeric binding molecule is a dimeric binding molecule comprising two bivalent IgA or IgA-like binding units. In some embodiments, each IgA heavy chain constant region or multimerizing fragment or variant thereof further comprises a Cα1 domain, a Cα2 domain, an IgA hinge region, or any combination thereof. In some embodiments, the IgA heavy chain constant regions or multimerizing fragments or variants thereof are human IgA constant regions. In some embodiments, each binding unit comprises two IgA heavy chains each comprising a VH situated amino terminal to the IgA constant region or multimerizing fragment thereof, and two immunoglobulin light chains each comprising a VL situated amino terminal to an immunoglobulin light chain constant region.

In some embodiments, the multimeric binding molecule comprises five bivalent IgM or IgM-like binding units, where each binding unit comprises two IgM heavy chain constant regions or multimerizing fragments or variants thereof, each comprising an IgM Cμ1 and IgM tailpiece domain. In some embodiments, each IgM heavy chain constant region or multimerizing fragment or variant thereof further comprises a Cμ1 domain, a Cμ2 domain, a Cμ3 domain, or any combination thereof. In some embodiments, the IgM heavy chain constant regions or multimerizing fragments or variants thereof are human IgM constant regions. In some embodiments, the IgM heavy chain constant regions each comprise the amino acid sequence SEQ ID NO: 1, SEQ ID NO: 2, or a multimerizing fragment or variant thereof.

In some embodiments, each binding unit comprises two IgM heavy chains each comprising a VH situated amino terminal to the IgM constant region or fragment thereof, and two immunoglobulin light chains each comprising a VL situated amino terminal to an immunoglobulin light chain constant region.

In some embodiments, the IgM constant regions each comprise one or more amino acid substitutions relative to a wild-type human IgM constant region at position 310, 311, 313, and/or 315 of SEQ ID NO: 1 or SEQ ID NO: 2, and where the multimeric binding molecule exhibits reduced complement-dependent cytotoxicity (CDC) activity to cells in the presence of complement, relative to a reference binding molecule that is identical except for the one or more amino acid substitutions.

In some embodiments, the IgM constant regions each comprise one or more substitutions relative to a wild-type human IgM constant region at positions 46, 209, 272, or 440 of SEQ ID NO: 1 or SEQ ID NO: 2, where the one or more amino acid substitutions prevent asparagine (N)-linked glycosylation.

In some embodiments, the J-chain or functional fragment or variant thereof comprises SEQ ID NO: 7.

In some embodiments, the J-chain or functional fragment or variant thereof comprises a variant J-chain or fragment thereof comprising one or more single amino acid substitutions, deletions, or insertions relative to a wild-type J-chain that can affect serum half-life of the binding molecule; and where the binding molecule exhibits an increased serum half-life upon administration to an animal relative to a reference binding molecule that is identical except for the one or more single amino acid substitutions, deletions, or insertions in the J-chain, and is administered in the same way to the same animal species. In some embodiments, the J-chain or functional fragment or variant thereof comprises an amino acid substitution at the amino acid position corresponding to amino acid Y102 of the mature wild-type human J-chain (SEQ ID NO: 7). In some embodiments, the amino acid corresponding to Y102 of SEQ ID NO: 7 is substituted with alanine (A), serine (S), or arginine (R). In some embodiments, the amino acid corresponding to Y102 of SEQ ID NO: 7 is substituted with alanine (A). In some embodiments, the J-chain or functional fragment or variant thereof is a variant human J-chain and comprises the amino acid sequence SEQ ID NO: 8 (“J*”).

In some embodiments, the J-chain or functional fragment or variant thereof further comprises the one or more effector moieties, where the one or more effector moieties is directly or indirectly fused to the J-chain or functional fragment or variant thereof. In some embodiments, the one or more effector moieties are fused to the J-chain or fragment thereof via a peptide linker. In some embodiments, the peptide linker comprises at least 5 amino acids, but no more than 25 amino acids. In some embodiments, the peptide linker consists of SEQ ID NO: 9, SEQ ID NO: 10, SEQ ID NO: 11, SEQ ID NO: 12, or SEQ ID NO: 13. In some embodiments, the one or more effector moieties are fused to the N-terminus of the J-chain or fragment or variant thereof, the C-terminus of the J-chain or fragment or variant thereof, or to both the N-terminus and C-terminus of the J-chain or fragment or variant thereof.

In some embodiments, the J-chain or fragment or variant thereof comprises SEQ ID NO: 32, SEQ ID NO: 33, SEQ ID NO: 36, SEQ ID NO:37, SEQ ID NO: 40, SEQ ID NO: 41, SEQ ID NO: 44, SEQ ID NO: 45, SEQ ID NO: 48, SEQ ID NO: 49, SEQ ID NO: 52, or SEQ ID NO: 53.

In some embodiments, the J-chain or fragment or variant thereof further comprises a heterologous polypeptide. In some embodiments, the heterologous polypeptide is an albumin or an albumin binding domain. In some embodiments, the heterologous polypeptide comprises human serum albumin. In some embodiments, the J-chain or fragment or variant thereof comprises SEQ ID NO: 34, SEQ ID NO: 38, SEQ ID NO: 42, SEQ ID NO: 46, SEQ ID NO: 50, or SEQ ID NO: 54.

In some embodiments, the subject is human.

### BRIEF DESCRIPTION OF THE DRAWINGS/FIGURES

FIG. 1A models the effector cell stimulation of standard bispecific effector cell engaging antibodies. FIG. 1B models the effector cell stimulation of an exemplary effector cell engaging molecule described herein.

FIG. 2A depicts the dose escalation plan of the phase 1 clinical trial. FIG. 2B shows the number of patients that have continued or discontinued treatment.

FIG. 3 shows the C-reactive protein (CRP) levels over time for a patient treated with mg IGM-2323. The dotted lines indicate when IGM-2323 was administered.

FIG. 4A shows the best response as percent change in sum of the products of tumor diameters (SPD) for each patient as measured by PET-CT scanning. The dosages shown are the patient's assigned dose level. * denotes that patient had received prior CAR-T therapy.

FIGS. 4B-4C show anti-CD3 immunohistochemistry (IHC) images of pre-treatment tumor tissue and a new PET-avid lesion 8 weeks post-treatment.

FIG. 5A shows the IFNγ plasma concentrations obtained during the sampling period among 14 of 16 patients treated in the study, the highest detected levels are shown with circles, the box plots show 1st and 3rd quartile, and the line connects mean values at each timepoint.

FIG. 5B shows the post-infusion peak IFNγ, TNFα, and IL-6 levels for the first and second treatment cycles among 14 of 16 patients treated in the study. The highest detected levels are shown with circles and the box plots show 1st and 3rd quartile.

FIGS. 5C-5E show post-infusion peak IFNγ, TNFα, and IL-6 levels for three different patients dosed with 30 mg IGM-2323.

FIGS. 6A-6B are reproduced figures from Brouwer-Visser et al. (25^{th} European Hematology Association (EHA) Virtual Congress, Jun. 11-21, 2020, poster EP1270) showing peak IFNγ (FIG. 6A) and IL-6 (FIG. 6B) after treatment with odronextamab. The solid black line denotes the mean concentration.

FIGS. 6C-6D are reproduced figures from Hernandez et al. (61^{st} ASH Annual Meeting & Exposition, Dec. 7-10, 2019, Orlando, Florida, USA, poster P-1585) showing peak IFNγ (FIG. 6C) and IL-6 (FIG. 6D) after treatment with mosunetuzumab.

FIG. 7 depicts the dose escalation plan of the phase 1 clinical trial as of Sep. 28, 2021.

FIG. 8 shows the best response as percent change in sum of the products of tumor diameters (SPD) for each patient as measured by PET-CT scanning for all patients.

FIG. 9 shows the best response as percent change in sum of the products of tumor diameters (SPD) for each patient as measured by PET-CT scanning for alternative dosing patients.

FIG. 10 shows response timelines for 11 patients responding to treatment with IGM-2323.

FIGS. 11A-11B show tumor responses as a percentage change from baseline for aggressive (FIG. 11A) and indolent (FIG. 11B) NHL.

FIG. 12A-12D shows the concentration of IGM-2323 in the blood over time for the 30 (FIG. 12A), 50/100 (FIG. 12B), 50/300 (FIG. 12C), and 50/600 (FIG. 12D) cohorts.

FIGS. 13A-13B show peak cytokine levels over cycle 1, 2, and 3+(FIG. 13A) or for cytokine release syndrome (CRS) patients vs. patients without CRS symptoms (FIG. 13B).

FIGS. 14A-14B show IGM-2323 binding to Ramos cells in the presence of various concentrations of rituximab after 5 mins (FIG. 14A) or 1 hour (FIG. 14B).

FIGS. 15A-15B show IGM-2323 binding to rituximab-resistant Ramos cells in the presence of various concentrations of rituximab after 5 mins (FIG. 15A) or 1 hour (FIG. 15B).

FIGS. 16A-16C show the percent inhibition of IGM-2323 in binding Ramos (FIG. 16A), CA46 (FIG. 16B), and rituximab-resistant Ramos (FIG. 16C) cells in the presence of various concentrations of rituximab.

FIG. 17A shows the amount of various concentrations of rituximab binding to Ramos cells with or without the presence of IGM-2323 after 5 min or 1 hour. FIG. 17B shows the % inhibition of various concentrations of rituximab in binding Ramos cells in the presence of IGM-2323.

FIG. 18 shows the percent inhibition of CD20×CD3 IgG in binding Ramos cells in the presence of various concentrations of rituximab.

FIG. 19 shows the percent of cells killed by complement dependent cytotoxicity (CDC) for 200 μg/mL IGM-2323 alone and with 2 μg/mL rituximab and for 2 μg/mL rituximab alone.

FIGS. 20A-20C show the percent of cells killed by complement dependent cytotoxicity (CDC) for 200 μg/mL IGM-2323 with various concentrations of rituximab and for various concentrations of rituximab alone on Ramos (FIG. 20A), CA46 (FIG. 20B), and rituximab-resistant Ramos (FIG. 20C) cells.

FIGS. 21A-21B show the percent of cells killed by complement dependent cytotoxicity (CDC) for 200 μg/mL IGM-2323 alone and with 200 μg/mL rituximab or with 200 μg/mL rituximab alone on CA46 (FIG. 21A) and rituximab-resistant Ramos (FIG. 21B) cells.

FIGS. 22A-22B show the percent of Ramos cells killed by T cell dependent cellular cytotoxicity (TDCC) for T cell donor 0585 (FIG. 22A) and donor 6369 (FIG. 22B) with various concentration of IGM-2323 and rituximab.

FIGS. 23A-23B show the percent of T cell dependent cellular cytotoxicity (TDCC) killing of Ramos cells for T cell donor 0585 (FIG. 22A) and donor 6369 (FIG. 22B) with various concentration of CD20×CD3 IgG and rituximab.

FIG. 24 shows the percent change in TDCC maximum killing activity (Emax) from 0 to 200 ug/mL rituximab for IGM-2323 and CD20×CD3 IgG.

FIGS. 25A-25B shows the percent of CD69 positive CD8 T cells for T cell donor 0585 (FIG. 25A) and donor 6369 (FIG. 25B) with various concentration of IGM-2323 and rituximab.

FIGS. 26A-26C shows the percent change in maximum percentage of CD69 (FIG. 26A), CD25 (FIG. 26B), and PD-1 (FIG. 26C) positive cells (Emax) from 0 to 200 ug/mL rituximab for IGM-2323 and CD20×CD3 IgG.

### DETAILED DESCRIPTION

### Definitions

As used herein, the term “a” or “an” entity refers to one or more of that entity; for example, “a binding molecule,” is understood to represent one or more binding molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various embodiments or embodiments of the disclosure, which can be had by reference to the specification as a whole. Accordingly, the terms defined immediately below are more fully defined by reference to the specification in its entirety.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of any of these terms. The term “polypeptide” is also intended to refer to the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology but is not necessarily translated from a designated nucleic acid sequence. It can be generated in any manner, including by chemical synthesis.

A polypeptide as disclosed herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt many different conformations and are referred to as unfolded. As used herein, the term glycoprotein refers to a protein coupled to at least one carbohydrate moiety that is attached to the protein via an oxygen-containing or a nitrogen-containing side chain of an amino acid, e.g., a serine or an asparagine. Asparagine (N)-linked glycans are described in more detail elsewhere in this disclosure.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are native or recombinant polypeptides which have been separated, fractionated, or partially or substantially purified by any suitable technique.

As used herein, the term “a non-naturally occurring polypeptide” or any grammatical variants thereof, is a conditional definition that explicitly excludes, but only excludes, those forms of the polypeptide that are, or might be, determined or interpreted by a judge or an administrative or judicial body, to be “naturally-occurring.”

Other polypeptides disclosed herein are fragments, derivatives, analogs, or variants of the foregoing polypeptides, and any combination thereof. The terms “fragment,” “variant,” “derivative” and “analog” as disclosed herein include any polypeptides which retain at least some of the properties of the corresponding native antibody or polypeptide, for example, specifically binding to an antigen. Fragments of polypeptides include, for example, proteolytic fragments, as well as deletion fragments, in addition to specific antibody fragments discussed elsewhere herein. Variants of, e.g., a polypeptide include fragments as described above, and also polypeptides with altered amino acid sequences due to amino acid substitutions, deletions, or insertions. In certain embodiments, variants can be non-naturally occurring. Non-naturally occurring variants can be produced using art-known mutagenesis techniques. Variant polypeptides can comprise conservative or non-conservative amino acid substitutions, deletions, or additions. Derivatives are polypeptides that have been altered so as to exhibit additional features not found on the original polypeptide. Examples include fusion proteins. As used herein a “derivative” of a polypeptide can also refer to a subject polypeptide having one or more amino acids chemically derivatized by reaction of a functional side group. Also included as “derivatives” are those polypeptides that contain one or more derivatives of the twenty standard amino acids. For example, 4-hydroxyproline can be substituted for proline; can be substituted for lysine; 3-methylhistidine can be substituted for histidine; homoserine can be substituted for serine; and ornithine can be substituted for lysine.

As used herein, the term “binding molecule” refers in its broadest sense to a molecule that specifically binds to a receptor or target, e.g., an epitope or an antigenic determinant. As described further herein, a binding molecule can comprise one of more “binding domains,” e.g., “antigen-binding domains” described herein. A non-limiting example of a binding molecule is an antibody or antibody-like molecule as described in detail herein that retains antigen-specific binding. In certain embodiments a “binding molecule” comprises an antibody or antibody-like or antibody-derived molecule as described in detail herein.

As used herein, the terms “binding domain” or “antigen-binding domain” (can be used interchangeably) refer to a region of a binding molecule, e.g., an antibody or antibody-like, or antibody-derived molecule, that is necessary and sufficient to specifically bind to a target, e.g., an epitope, a polypeptide, a cell, or an organ. For example, an “Fv,” e.g., a heavy chain variable region and a light chain variable region of an antibody, either as two separate polypeptide subunits or as a single chain, is considered to be a “binding domain.” Other antigen-binding domains include, without limitation, a single domain heavy chain variable region (VHH) of an antibody derived from a camelid species, or six immunoglobulin complementarity determining regions (CDRs) expressed in a fibronectin scaffold. A “binding molecule,” e.g., an “antibody” as described herein can include one, two, three, four, five, six, seven, eight, nine, ten, eleven, twelve, or more “antigen-binding domains.”

The terms “antibody” and “immunoglobulin” can be used interchangeably herein. An antibody (or a fragment, variant, or derivative thereof as disclosed herein, e.g., an IgM-like antibody) includes at least the variable domain of a heavy chain (e.g., from a camelid species) or at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988). Unless otherwise stated, the term “antibody” encompasses anything ranging from a small antigen-binding fragment of an antibody to a full sized antibody, e.g., an IgG antibody that includes two complete heavy chains and two complete light chains, an IgA antibody that includes four complete heavy chains and four complete light chains and includes a J-chain and/or a secretory component, or an IgM-derived binding molecule, e.g., an IgM antibody or IgM-like antibody, that includes ten or twelve complete heavy chains and ten or twelve complete light chains and optionally includes a J-chain or functional fragment or variant thereof.

The term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4 or α1-α2)). It is the nature of this chain that determines the “isotype” of the antibody as IgG, IgM, IgA IgD, or IgE, respectively. The immunoglobulin subclasses (subtypes) e.g., IgG₁, IgG₂, IgG₃, IgG₄, IgA₁, IgA₂, etc. are well characterized and are known to confer functional specialization. Modified versions of each of these immunoglobulins are readily discernible to the skilled artisan in view of the instant disclosure and, accordingly, are within the scope of this disclosure.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are expressed, e.g., by hybridomas, B cells or genetically engineered host cells. In the heavy chain, the amino acid sequences run from an N-terminus at the forked ends of the Y configuration to the C-terminus at the bottom of each chain. The basic structure of certain antibodies, e.g., IgG antibodies, includes two heavy chain subunits and two light chain subunits covalently connected via disulfide bonds to form a “Y” structure, also referred to herein as an “H2L2” structure, or a “binding unit.”

The term “binding unit” is used herein to refer to the portion of a binding molecule, e.g., an antibody, antibody-like molecule, or antibody-derived molecule, antigen-binding fragment thereof, or multimerizing fragment thereof, which corresponds to a standard “H2L2” immunoglobulin structure, i.e., two heavy chains or fragments thereof and two light chains or fragments thereof. In certain embodiments, e.g., where the binding molecule is a bivalent IgG antibody or antigen-binding fragment thereof, the terms “binding molecule” and “binding unit” are equivalent. Such binding molecules are also referred to herein as “monomeric.” In other embodiments, e.g., where the binding molecule is a “multimeric binding molecule,” e.g., a dimeric or tetrameric IgA antibody, a dimeric or tetrameric IgA-like antibody, a dimeric or tetrameric IgA-derived binding molecule, a pentameric or hexameric IgM antibody, a pentameric or hexameric IgM-like antibody, or a pentameric or hexameric IgM-derived binding molecule or any derivative thereof, the binding molecule comprises two or more “binding units.” Two in the case of an IgA dimer, four in the case of an IgA tetramer, or five or six in the case of an IgM pentamer or hexamer, respectively. A binding unit need not include full-length antibody heavy and light chains, but will typically be bivalent, i.e., will include two “antigen-binding domains,” as defined above. As used herein, certain binding molecules provided in this disclosure are “dimeric,” and include two bivalent binding units that include IgA constant regions or multimerizing fragments thereof. Certain binding molecules provided in this disclosure are “pentameric” or “hexameric,” and include five or six bivalent binding units that include IgM constant regions or multimerizing fragments or variants thereof. A binding molecule, e.g., an antibody or antibody-like molecule or antibody-derived binding molecule, comprising two or more, e.g., two, five, or six binding units, is referred to herein as “multimeric.”

The term “J-chain” as used herein refers to the J-chain of IgM or IgA antibodies of any animal species, any functional fragment thereof, derivative thereof, and/or variant thereof, including a mature human J-chain, the amino acid sequence of which is presented as SEQ ID NO: 7. Various J-chain variants and modified J-chain derivatives are disclosed herein. As persons of ordinary skill in the art will recognize, “a functional fragment” or “a functional variant” includes those fragments and variants that can associate with IgM heavy chain constant regions to form a pentameric IgM antibody or can associate with IgA heavy chain constant regions to form a dimeric IgA antibody.

The term “modified J-chain” is used herein to refer to a derivative of a J-chain polypeptide comprising a heterologous moiety, e.g., a heterologous polypeptide, e.g., an extraneous binding domain or functional domain introduced into or attached to the J-chain sequence. The introduction can be achieved by any means, including direct or indirect fusion of the heterologous polypeptide or other moiety or by attachment through a peptide or chemical linker. The term “modified human J-chain” encompasses, without limitation, a native sequence human J-chain comprising the amino acid sequence of SEQ ID NO: 7 or functional fragment thereof, or functional variant thereof, modified by the introduction of a heterologous moiety, e.g., a heterologous polypeptide, e.g., an extraneous binding domain. In certain embodiments the heterologous moiety does not interfere with efficient polymerization of IgM into a pentamer or IgA into a multimer, e.g., a dimer or tetramer, and binding of such polymers to a target. Exemplary modified J-chains can be found, e.g., in U.S. Pat. Nos. 9,951,134 and 10,400,038, and in U.S. Patent Application Publication Nos. US-2019-0185570 and US-2018-0265596, each of which is incorporated herein by reference in its entirety.

As used herein the term “IgM-derived binding molecule” refers collectively to native IgM antibodies, IgM-like antibodies, as well as other IgM-derived binding molecules comprising non-antibody binding and/or functional domains instead of an antibody antigen binding domain or subunit thereof, and any fragments, e.g., multimerizing fragments, variants, or derivatives thereof.

As used herein, the term “IgM-like antibody” refers generally to a variant antibody or antibody-derived binding molecule that still retains the ability to form hexamers or pentamers, e.g., in association with a J-chain. An IgM-like antibody or other IgM-derived binding molecule typically includes at least the Cμ4-tp domains of the IgM constant region but can include heavy chain constant region domains from other antibody isotypes, e.g., IgG, from the same species or from a different species. An IgM-like antibody or other IgM-derived binding molecule can likewise be an antibody fragment in which one or more constant regions are deleted, as long as the IgM-like antibody is capable of forming hexamers and/or pentamers. Thus, an IgM-like antibody or other IgM-derived binding molecule can be, e.g., a hybrid IgM/IgG antibody or can be a “multimerizing fragment” of an IgM antibody.

As used herein the term “IgA-derived binding molecule” refers collectively to native IgA antibodies, IgA-like antibodies, as well as other IgA-derived binding molecules comprising non-antibody binding and/or functional domains instead of an antibody antigen binding domain or subunit thereof, and any fragments, e.g., multimerizing fragments, variants, or derivatives thereof.

As used herein, the term “IgA-like antibody” refers generally to a variant antibody or antibody-derived binding molecule that still retains the ability to form multimers, e.g., dimers, trimers, tetramers, and/or pentamers e.g., dimers and/or tetramers, e.g., in association with a J-chain. An IgA-like antibody or other IgA-derived binding molecule typically includes at least the Cα3-tp domains of the IgA constant region but can include heavy chain constant region domains from other antibody isotypes, e.g., IgG, from the same species or from a different species. An IgA-like antibody or other IgA-derived binding molecule can likewise be an antibody fragment in which one or more constant regions are deleted, as long as the IgA-like antibody is capable of forming multimers, e.g., dimers and/or tetramers. Thus, an IgA-like antibody or other IgA-derived binding molecule can be, e.g., a hybrid IgA/IgG antibody or can be a “multimerizing fragment” of an IgA antibody.

The terms “valency,” “bivalent,” “multivalent” and grammatical equivalents, refer to the number of binding domains, e.g., antigen-binding domains in given binding molecule, e.g., antibody, antibody-derived, or antibody-like molecule, or in a given binding unit. As such, the terms “bivalent”, “tetravalent”, and “hexavalent” in reference to a given binding molecule, e.g., an IgM antibody, IgM-like antibody, other IgM-derived binding molecule, or multimerizing fragment thereof, denote the presence of two antigen-binding domains, four antigen-binding domains, and six antigen-binding domains, respectively. A typical IgM antibody, IgM-like antibody, or other IgM-derived binding molecule, where each binding unit is bivalent, can have 10 or 12 valencies. A bivalent or multivalent binding molecule, e.g., antibody or antibody-derived molecule, can be monospecific, i.e., all of the antigen-binding domains are the same, or can be bispecific or multispecific, e.g., where two or more antigen-binding domains are different, e.g., bind to different epitopes on the same antigen, or bind to entirely different antigens.

The term “epitope” includes any molecular determinant capable of specific binding to an antigen-binding domain of an antibody, antibody-like, or antibody-derived molecule. In certain embodiments, an epitope can include chemically active surface groupings of molecules such as amino acids, sugar side chains, phosphoryl groups, or sulfonyl groups, and, in certain embodiments, can have three-dimensional structural characteristics, and or specific charge characteristics. An epitope is a region of a target that is bound by an antigen-binding domain of an antibody.

The term “target” is used in the broadest sense to include substances that can be bound by a binding molecule, such as an effector cell engaging molecule, e.g., antibody or antibody-like molecule. A target can be, e.g., a polypeptide, a nucleic acid, a carbohydrate, a lipid, or other molecule. Moreover, a “target” can, for example, be a cell, an organ, or an organism that comprises an epitope that can be bound by a binding molecule, such as an effector cell engaging molecule, e.g., antibody or antibody-like molecule. As used herein, a “target antigen” is a target molecule, e.g., a polypeptide, a nucleic acid, a carbohydrate, a lipid, or other molecule that can be bound by a binding molecule, such as an effector cell engaging molecule, e.g., an antibody or antibody-like molecule as provided herein. In certain embodiments a target antigen can appear on the surface of a cell, e.g., a tumor cell. A “tumor-specific antigen” as used herein is a protein or other cell surface target antigen that is unique to tumor cells, at least at later stages of development of the organism. As used herein, a “tumor-associated antigen” is a protein or other cell surface target antigen that is not necessarily unique to tumor cells but is typically expressed much more abundantly and/or at higher density on tumor cells than on normal, healthy cells.

Both the light and heavy chains of antibodies, antibody-like, or antibody-derived molecules are divided into regions of structural and functional homology. The terms “constant” and “variable” are used functionally. In this regard, it will be appreciated that the variable domains of both the variable light (VL) and variable heavy (VH) chain portions determine antigen recognition and specificity. Conversely, the constant region domains of the light chain (CL) and the heavy chain (e.g., CH1, CH2, CH3, or CH4) confer biological properties such as secretion, transplacental mobility, Fc receptor binding, complement binding, and the like. By convention, the numbering of the constant region domains increases as they become more distal from the antigen-binding site or amino-terminus of the antibody. The N-terminal portion is a variable region and at the C-terminal portion is a constant region; the CH3 (or CH4, e.g., in the case of IgM) and CL domains actually comprise the carboxy-terminus of the heavy and light chain, respectively.

A “full length IgM antibody heavy chain” is a polypeptide that includes, in N-terminal to C-terminal direction, an antibody heavy chain variable domain (VH), an antibody heavy chain constant domain 1 (CM1 or Cμ1), an antibody heavy chain constant domain 2 (CM2 or Cμ2), an antibody heavy chain constant domain 3 (CM3 or Cμ3), and an antibody heavy chain constant domain 4 (CM4 or Cμ4) that can include a tailpiece.

A “full length IgA antibody heavy chain” is a polypeptide that includes, in N-terminal to C-terminal direction, an antibody heavy chain variable domain (VH), an antibody heavy chain constant domain 1 (CA1 or Cα1), an IgA hinge region, an antibody heavy chain constant domain 2 (CA2 or Cα2), and an antibody heavy chain constant domain 3 (CA3 or Cα3) that can include an IgA tailpiece.

As indicated above, variable region(s) allow a binding molecule, e.g., antibody, antibody-like, or antibody-derived molecule, to selectively recognize and specifically bind epitopes on antigens. That is, the VL domain and VH domain, or subset of the complementarity determining regions (CDRs), of a binding molecule, e.g., an antibody, antibody-like, or antibody-derived molecule, combine to form the antigen-binding domain. More specifically, an antigen-binding domain can be defined by three CDRs on each of the VH and VL chains. Certain antibodies form larger structures. For example, IgA can form a molecule that includes two H2L2 binding units and a J-chain covalently connected via disulfide bonds, which can be further associated with a secretory component, and IgM can form a pentameric or hexameric molecule that includes five or six H2L2 binding units and optionally a J-chain covalently connected via disulfide bonds.

The six “complementarity determining regions” or “CDRs” present in an antibody antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domain, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids that make up the CDRs and the framework regions, respectively, can be readily identified for any given heavy or light chain variable region by one of ordinary skill in the art, since they have been defined in various different ways (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. These particular regions have been described, for example, by Kabat et al., U.S. Dept. of Health and Human Services, “Sequences of Proteins of Immunological Interest” (1983) and by Chothia et al., J. Mol. Biol. 196:901-917 (1987), which are incorporated herein by reference. The Kabat and Chothia definitions include overlapping or subsets of amino acids when compared against each other. Nevertheless, application of either definition (or other definitions known to those of ordinary skill in the art) to refer to a CDR of an antibody or variant thereof is intended to be within the scope of the term as defined and used herein, unless otherwise indicated. The appropriate amino acids which encompass the CDRs as defined by each of the above cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody.

TABLE 1
CDR Definitions*

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

*Numbering of all CDR definitions in Table 1 is according to the numbering conventions set forth by Kabat et al. (see below).

Antibody variable domains can also be analyzed, e.g., using the IMGT information system (imgt_dot_cines_dot_fr/) (IMGT®/V-Quest) to identify variable region segments, including CDRs. (See, e.g., Brochet et al., Nucl. Acids Res. 36:W503-508, 2008).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless use of the Kabat numbering system is explicitly noted, however, consecutive numbering is used for all amino acid sequences in this disclosure.

The Kabat numbering system for the human IgM constant domain can be found in Kabat, et. al. “Tabulation and Analysis of Amino acid and nucleic acid Sequences of Precursors, V-Regions, C-Regions, J-Chain, T-Cell Receptors for Antigen, T-Cell Surface Antigens, β-2 Microglobulins, Major Histocompatibility Antigens, Thy-1, Complement, C-Reactive Protein, Thymopoietin, Integrins, Post-gamma Globulin, α-2 Macroglobulins, and Other Related Proteins,” U.S. Dept. of Health and Human Services (1991). IgM constant regions can be numbered sequentially (i.e., amino acid #1 starting with the first amino acid of the constant region, or by using the Kabat numbering scheme. A comparison of the numbering of two alleles of the human IgM constant region sequentially (presented herein as SEQ ID NO: 1 (allele IGHM*03) and SEQ ID NO: 2 (allele IGHM*04)) and by the Kabat system is set out below. The underlined amino acid residues are not accounted for in the Kabat system (“X,” double underlined below, can be serine (S) (SEQ ID NO: 1) or glycine (G) (SEQ ID NO: 2)):

| Sequential (SEQ ID NO: 1 or SEQ ID NO: 2)/KABAT numbering key | |
|---|---|
| for IgM heavy chain | |
| 1/127 | GSASAPTLFP LVSCENSPSD TSSVAVGCLA QDELPDSITF SWKYKNNSDI |
| 51/176 | SSTRGFPSVL RGGKYAATSQ VLLPSKDVMQ GTDEHVVCKV QHPNGNKEKN |
| 101/226 | VPLPVIAELP PKVSVFVPPR DGFFGNPRKS KLICQATGES PRQIQVSWLR |
| 151/274 | EGKQVGSGVT TDQVQAEAKE SGPTTYKVTS TLTIKESDWL XQSMFTCRVD |
| 201/324 | HRGLTFQQNA SSMCVPDQDT AIRVFAIPPS FASIFLTKST KLTCLVTDLT |
| 251/374 | TYDSVTISWT RQNGEAVKTH TNISESHPNA TFSAVGEASI CEDDWNSGER |
| 301/424 | FTCTVTHTDL PSPLKQTISR PKGVALHRPD VYLLPPAREQ LNLRESATIT |
| 351/474 | CLVTGFSPAD VEVQWMQRGQ PLSPEKYVTS APMPEPQAPG RYFAHSILTV |
| 401/524 | SEEEWNTGET YTCVVAHEAL PNRVTERTVD KSTGKPTLYN VSLVMSDTAG |
| 451/574 | TCY |

Binding molecules, e.g., antibodies, antibody-like, or antibody-derived molecules, antigen-binding fragments, variants, or derivatives thereof, and/or multimerizing fragments thereof include, but are not limited to, polyclonal, monoclonal, human, humanized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019.

By “specifically binds,” it is generally meant that a binding molecule, e.g., an antibody or fragment, variant, or derivative thereof binds to an epitope via its antigen-binding domain, and that the binding entails some complementarity between the antigen-binding domain and the epitope. According to this definition, a binding molecule, e.g., antibody, antibody-like, or antibody-derived molecule, is said to “specifically bind” to an epitope when it binds to that epitope, via its antigen-binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

As used herein, the term “affinity” refers to a measure of the strength of the binding of an individual epitope with one or more antigen-binding domains, e.g., of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of antigen-binding domains and an antigen. See, e.g., Harlow at pages 29-34. Avidity is related to both the affinity of individual antigen-binding domains in the population with specific epitopes, and also the valencies of the immunoglobulins and the antigen. For example, the interaction between a bivalent monoclonal antibody and an antigen with a highly repeating epitope structure, such as a polymer, would be one of high avidity. An interaction between a bivalent monoclonal antibody with a receptor present at a high density on a cell surface would also be of high avidity.

Binding molecules, e.g., antibodies or fragments, variants, or derivatives thereof as disclosed herein can also be described or specified in terms of their cross-reactivity. As used herein, the term “cross-reactivity” refers to the ability of a binding molecule, e.g., an antibody or fragment, variant, or derivative thereof, specific for one antigen, to react with a second antigen; a measure of relatedness between two different antigenic substances. Thus, a binding molecule is cross reactive if it binds to an epitope other than the one that induced its formation. The cross-reactive epitope generally contains many of the same complementary structural features as the inducing epitope, and in some cases, can actually fit better than the original.

“Antigen-binding antibody fragments” including single-chain antibodies or other antigen-binding domains can exist alone or in combination with one or more of the following: hinge region, CH1, CH2, CH3, or CH4 domains, J-chain, or secretory component. Also included are antigen-binding fragments that can include any combination of variable region(s) with one or more of a hinge region, CH1, CH2, CH3, or CH4 domains, a J-chain, or a secretory component. Binding molecules, e.g., antibodies, or antigen-binding fragments thereof can be from any animal origin including birds and mammals. The antibodies can be human, murine, donkey, rabbit, goat, guinea pig, camel, llama, horse, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and can in some instances express endogenous immunoglobulins and some not, as described infra and, for example in, U.S. Pat. No. 5,939,598 by Kucherlapati et al. According to embodiments of the present disclosure, an IgM antibody, IgM-like antibody, or other IgM-derived binding molecule as provided herein can include an antigen-binding fragment of an antibody, e.g., a scFv fragment, so long as the IgM antibody, IgM-like antibody, or other IgM-derived binding molecule is able to form a multimer, e.g., a hexamer or a pentamer, and an IgA antibody, IgA-like antibody, or other IgA-derived binding molecule as provided herein can include an antigen-binding fragment of an antibody, e.g., a scFv fragment, so long as the IgA antibody, IgA-like antibody, or other IgA-derived binding molecule is able to form a multimer, e.g., a dimer and/or a tetramer. As used herein such a fragment comprises a “multimerizing fragment.”

As used herein, the term “heavy chain subunit” includes amino acid sequences derived from an immunoglobulin heavy chain, a binding molecule, e.g., an antibody, antibody-like, or antibody-derived molecule comprising a heavy chain subunit can include at least one of: a VH domain, a CH1 domain, a hinge (e.g., upper, middle, and/or lower hinge region) domain, a CH2 domain, a CH3 domain, a CH4 domain, or a variant or fragment thereof. For example, a binding molecule, e.g., an antibody, antibody-like, or antibody-derived molecule, or fragment, e.g., multimerizing fragment, variant, or derivative thereof can include without limitation, in addition to a VH domain: a CH1 domain; a CH1 domain, a hinge, and a CH2 domain; a CH1 domain and a CH3 domain; a CH1 domain, a hinge, and a CH3 domain; or a CH1 domain, a hinge domain, a CH2 domain, and a CH3 domain. In certain embodiments a binding molecule, e.g., an antibody, antibody-like, or antibody-derived molecule, or fragment, e.g., multimerizing fragment, variant, or derivative thereof can include, in addition to a VH domain, a CH3 domain and a CH4 domain; or a CH3 domain, a CH4 domain, and a J-chain. Further, a binding molecule, e.g., an antibody, antibody-like, or antibody-derived molecule, for use in the disclosure can lack certain constant region portions, e.g., all or part of a CH2 domain. It will be understood by one of ordinary skill in the art that these domains (e.g., the heavy chain subunit) can be modified such that they vary in amino acid sequence from the original immunoglobulin molecule. According to embodiments of the present disclosure, an IgM antibody, IgM-like antibody, or other IgM-derived binding molecule as provided herein comprises sufficient portions of an IgM heavy chain constant region to allow the IgM antibody, IgM-like antibody, or other IgM-derived binding molecule to form a multimer, e.g., a hexamer or a pentamer. As used herein such a fragment comprises a “multimerizing fragment.” According to embodiments of the present disclosure, an IgA antibody, IgA-like antibody, or other IgA-derived binding molecule as provided herein comprises sufficient portions of an IgA heavy chain constant region to allow the IgA antibody, IgA-like antibody, or other IgA-derived binding molecule to form a multimer, e.g., a dimer or a tetramer. As used herein such a fragment comprises a “multimerizing fragment.”

As used herein, the term “light chain subunit” includes amino acid sequences derived from an immunoglobulin light chain. The light chain subunit includes at least a VL, and can further include a CL (e.g., Cκ or Cλ) domain.

Binding molecules, e.g., antibodies, antibody-like molecules, antibody-derived molecules, antigen-binding fragments, variants, or derivatives thereof, or multimerizing fragments thereof can be described or specified in terms of the epitope(s) or portion(s) of a target, e.g., a target antigen that they recognize or specifically bind. The portion of a target antigen that specifically interacts with the antigen-binding domain of an antibody is an “epitope,” or an “antigenic determinant.” A target antigen can comprise a single epitope or at least two epitopes, and can include any number of epitopes, depending on the size, conformation, and type of antigen.

As used herein, the term “chimeric antibody” refers to an antibody in which the immunoreactive region or site is obtained or derived from a first species and the constant region (which can be intact, partial, or modified) is obtained from a second species. In some embodiments the target binding region or site will be from a non-human source (e.g., mouse or primate) and the constant region is human.

The terms “multispecific antibody” or “bispecific antibody” refer to an antibody, antibody-like, or antibody-derived molecule that has antigen-binding domains for two or more different epitopes within a single antibody molecule. Other binding molecules in addition to the canonical antibody structure can be constructed with two binding specificities. Epitope binding by bispecific or multispecific antibodies can be simultaneous or sequential. Triomas and hybrid hybridomas are two examples of cell lines that can secrete bispecific antibodies. Bispecific antibodies can also be constructed by recombinant means. (Strohlein and Heiss, Future Oncol. 6:1387-94 (2010); Mabry and Snavely, IDrugs. 13:543-9 (2010)). A bispecific antibody can also be a diabody.

As used herein, the term “engineered antibody” refers to an antibody in which a variable domain, constant region, and/or J-chain is altered by at least partial replacement of one or more amino acids. In certain embodiments entire CDRs from an antibody of known specificity can be grafted into the framework regions of a heterologous antibody. Although alternate CDRs can be derived from an antibody of the same class or even subclass as the antibody from which the framework regions are derived, CDRs can also be derived from an antibody of different class, e.g., from an antibody from a different species. An engineered antibody in which one or more “donor” CDRs from a non-human antibody of known specificity are grafted into a human heavy or light chain framework region is referred to herein as a “humanized antibody.” In certain embodiments not all of the CDRs are replaced with the complete CDRs from the donor variable region and yet the antigen-binding capacity of the donor can still be transferred to the recipient variable domains. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing, to obtain a functional engineered or humanized antibody.

As used herein the term “engineered” includes manipulation of nucleic acid or polypeptide molecules by synthetic means (e.g., by recombinant techniques, in vitro peptide synthesis, by enzymatic or chemical coupling of peptides, nucleic acids, or glycans, or some combination of these techniques).

As used herein, the terms “linked,” “fused” or “fusion” or other grammatical equivalents can be used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence. For example, polynucleotides encoding the CDRs of an immunoglobulin variable region can be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which amino acids that neighbor each other in the sequence are contiguous in the primary structure of the polypeptide. A portion of a polypeptide that is “amino-terminal” or “N-terminal” to another portion of a polypeptide is that portion that comes earlier in the sequential polypeptide chain. Similarly, a portion of a polypeptide that is “carboxy-terminal” or “C-terminal” to another portion of a polypeptide is that portion that comes later in the sequential polypeptide chain. For example, in a typical antibody, the variable domain is “N-terminal” to the constant region, and the constant region is “C-terminal” to the variable domain.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into RNA, e.g., messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

The term “determined dosage” as used herein means a dosage of an effector cell engaging molecule that has been set by existing or predetermined criteria. A “determined dosage” can be, for example, the minimal or initial effective dosage of a given effector cell engaging molecule as determined in preclinical or clinical trials, or as noted on a product label. Alternatively, if a subject has been previously treated or is currently being treated with the effector cell engaging molecule, the “determined dosage” can be the most recent dosage administered to the subject.

The term “chimeric antigen receptor” or “CAR” refers to a recombinant molecule expressed on the surface of an effector cell, e.g., a T cell or a natural killer (NK) cell, that provides the effector cell with an ability to target a specific antigen, e.g., a tumor antigen, thereby activating the effector cell. A CAR typically includes an extracellular antigen-binding domain (e.g., an antibody scFv or a ligand), a transmembrane domain, and an intracellular effector cell signaling domain. See, e.g., Chandran, S S, and C A Klebanoff, Immunol. Rev. 290:127-147 (2019). The transmembrane domain can be derived, e.g., from CD28, CD8α, or CD3ζ. The intracellular region is derived from a protein capable of triggering effector cell activation, e.g., T cell activation. In T cells, this intracellular domain typically includes the CD3ζ endodomain and a costimulatory region, e.g., from CD28, CD27, CD134 (OX40), CD137 (4-1BB), CD154 (CD40L), CD278 (ICOS), or CD244 (2B4). In certain embodiments the CAR further includes a spacer or hinge region between the antigen-binding domain and the transmembrane domain to provide greater stability and flexibility to the antigen-binding domain. This spacer region can be derived from the membrane-proximal domain of a number of different cell-surface expressed proteins, e.g., CD8, CD28, or an immunoglobulin constant region. To produce CAR-expressing effector cells, typically a transgene encoding the CAR is introduced into cells isolated from the subject, e.g., by retroviral transduction. The recombinant cells are then introduced into the subject.

As used herein, the term “effector cell” refers to cells in the innate or adaptive immune system that are capable of responding to a stimulus, e.g., by differentiating, activating, and/or proliferating. The stimulus can be, e.g., association of the effector cell with a ligand. Effector cells include, without limitation, effector T cells, e.g., cytotoxic T cells (CTL) or T helper cells, plasma cells, and natural killer cells.

As used herein, the term “effector cell engaging molecule” refers to a binding molecule, e.g., a bispecific antibody, that binds to both an effector cell, e.g., a T cell or an NK cell, and to a target antigen on a target cell, e.g., a tumor antigen expressed on a tumor cell, thereby bringing the effector cell in close proximity to the target cell, allowing the effector cell to act on the target cell, e.g., killing the target cell.

As used herein, the term “avidity” refers to the overall stability of the complex between a population of antigen-binding domains and an antigen, or a complex of antigens. See, e.g., Harlow et al., Antibodies: A Laboratory Manual, (Cold Spring Harbor Laboratory Press, 2nd ed. 1988) at pages 29-34. Avidity is related to both the affinity of the individual antigen-binding domains in the population with specific epitopes, and the valencies of immunoglobulins and the antigen. For example, a decavalent IgM antibody would bind to an antigen with higher avidity than a bivalent IgG antibody with antigen-binding domains having equivalent binding affinities. Avidity can also be affected by the antigen—for example, the interaction between a bivalent IgG antibody and an antigen with a highly repeating epitope structure, such as a polymer, would be one of high avidity. An interaction between a bivalent, tetravalent, or decavalent antibody with a receptor present at a high density on a cell surface would also be of high avidity. Where an antibody is binding to a target antigen present on the surface of a cell, e.g., a tumor-specific or tumor-associated antigen on a tumor cell, at least three levers can affect avidity: (a) the valency of the antibody, e.g., a bivalent IgG antibody will have a different avidity than a decavalent IgM antibody with equivalent antigen-binding domains; (b) an increase or decrease in the affinity of individual antigen-binding domains of the antibody, e.g., two, three, four, five, six, seven, eight, nine, ten, eleven, or twelve antigen-binding domain, and/or (c) the density of the target antigen of the surface of the cell, e.g., the difference between a tumor cell which expresses a tumor-associated antigen at high density versus a normal healthy cell that expresses the target antigen at lower density. This disclosure provides methods that employ effector cell engaging molecules in which levers (a) and (b) can be manipulated to provide a more physiologic effector response.

As used herein, the “potency” of a binding molecule or a subunit thereof, e.g., an effector cell engaging molecule or an individual binding domain of an effector cell engaging molecule, is a measure of the molecule's activity expressed in terms of the amount required to produce a desired effect, either in vitro or in vivo. For example, a bivalent antibody specific for CD20 can have a specific potency for killing 50% of CD-20-expressing cells in an in vitro assay, expressed as an EC₅₀ measured either by mass/volume (e.g., in μg/ml) or as a molar concentration. Two different anti-CD20 antibodies can have vastly different potencies, meaning the EC₅₀s of the molecules in a given assay differ significantly. Potency can also refer to the amount of an immunotherapeutic molecule required to achieve a desired result in a subject treated with the molecule, e.g., the amount of the molecule required to slow or stop tumor progression, to reduce tumor volume, or to affect a complete anti-tumor response. An effector cell engaging molecule as provided herein can have one or more tumor targeting moieties and one or more effector cell engaging moieties, and therefore the molecule as a whole can have at least two potency measurements—one related to the ability of the molecule to bind to and kill tumor cells, and one related to the ability of the molecule to engage and activate effector cells. While the two potencies are related, e.g., CTL engagement can result in enhanced tumor cell killing, they can also be measured and compared separately, e.g., the ability of the effector cell engaging molecule to cause CTL to activate, e.g., produce cytokines, or proliferate.

As used herein, the term “cumulative potency” refers to the total potency of a given effector cell engaging molecule for a given target, where the effector cell engaging molecule can have one, two, three, four, five, six, seven, eight, nine, or ten or more binding domains (targeting moieties) that bind to the given target. An effector cell engaging molecule as provided herein can have one or more, e.g., up to ten tumor targeting moieties or binding domains and one or more effector cell engaging moieties or binding domains. Each tumor targeting moiety can have a given potency, e.g., each individual binding domain in the effector cell engaging molecule that targets the tumor cell, and each individual binding domain in the effector cell engaging molecule that targets the effector cell. The potencies of the two or more individual binding domains of an effector cell engaging molecule that targets the same antigen can result in an additive or a greater than additive potency of the effector cell engaging molecule for that antigen. For example, an exemplary pentameric IgM antibody as provided herein has ten individual binding domains that target CD20 on B cells and one binding domain that targets CD3 on T cells. The potency of a pentameric IgM antibody specific for CD20 much greater than five-fold the potency of a bivalent IgG antibody comprising two of the same CD20 binding domains in a variety of different in vitro and in vivo assays. See, e.g., U.S. Pat. No. 10,787,520. The used herein, the term “cumulative potency” refers to the total potency of a given effector cell engaging molecule for a given target. As noted above, a single effector cell engaging molecule can possess two different potencies, one for the tumor target and one for effector cell activation. These two potencies can be separately tuned, e.g., by increasing the avidity and/or affinity of the tumor targeting moieties or decreasing the affinity and/or avidity of the effector cell engaging moieties. The relative potencies could also be adjusted, e.g., by sterically hindering access of the effector cell targeting moieties for the effector cell or increasing or decreasing the distance or topology between the tumor cell and the effector cell.

As used herein, a “physiologic level” or “physiological level,” as it applies to effector cell activity denotes a level of activity that would be expected in a subject who is not receiving effector cell-targeting immunotherapy. For example, a “physiologic level” of CTL activity would be the level of CTL proliferation and cytokine expression that would be observed in a human subject during a mild rhinovirus infection. Such a response is effective to contain the virus infection, resulting, e.g., in a mild fever and aches, but quickly resolves. In contrast, treatment with certain immunotherapy drugs, e.g., CAR-T cells or certain bispecific antibodies, can result in a pathologic effector cell response, for example, cytokine release syndrome, which can include severe and even life-threatening symptoms.

The use of a generic name of a biologic therapeutic is understood to include any and all biosimilars bearing the generic name, unless specifically stated otherwise. For example, use of the term “rituximab” would include e.g., rituximab, rituximab-pvvr, and rituximab-abbs.

Terms such as “treating” or “treatment” or “to treat” or “alleviating” or “to alleviate” refer to therapeutic measures that cure, slow down, lessen symptoms of, lessen the severity of symptoms of, and/or halt or slow the progression of an existing diagnosed pathologic condition or disorder. Terms such as “prevent,” “prevention,” “avoid,” “deterrence,” “prophylactic,” and the like refer to prophylactic or preventative measures that prevent the development of an undiagnosed targeted pathologic condition or disorder. Thus, “those in need of treatment” can include those already with the disorder and/or those prone to have the disorder.

As used herein the terms “serum half-life” or “plasma half-life” refer to the time it takes (e.g., in minutes, hours, or days) following administration for the serum or plasma concentration of a drug, e.g., a binding molecule such as an antibody, antibody-like, or antibody-derived molecule or fragment, e.g., multimerizing fragment thereof as described herein, to be reduced by 50%. Two half-lives can be described: the alpha half-life, α half-life, or t_{1/2}α, which is the rate of decline in plasma concentrations due to the process of drug redistribution from the central compartment, e.g., the blood in the case of intravenous delivery, to a peripheral compartment (e.g., a tissue or organ), and the beta half-life, β half-life, or t_{1/2}β which is the rate of decline due to the processes of excretion or metabolism.

As used herein the term “area under the plasma drug concentration-time curve” or “AUC” reflects the actual body exposure to drug after administration of a dose of the drug and is expressed in mg*h/L. This area under the curve can be measured, e.g., from time 0 (t₀) to infinity (∞) and is dependent on the rate of elimination of the drug from the body and the dose administered.

As used herein, the term “mean residence time” or “MRT” refers to the average length of time the drug remains in the body.

By “subject” or “individual” or “animal” or “patient” or “mammal,” is meant any subject. In certain embodiments the subject is a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, swine, cows, bears, and so on.

As used herein, as the term “a subject that would benefit from therapy” refers to a subset of subjects, from amongst all prospective subjects, which would benefit from administration of a given therapeutic agent, e.g., a binding molecule such as an antibody, comprising one or more antigen-binding domains. Such binding molecules, e.g., antibodies, can be used, e.g., for a diagnostic procedure and/or for treatment or prevention of a disease.

As used herein, the terms “fever” and “pyrexia” are used interchangeably and refer to a symptom characterized by an elevation of a subject's body temperature above normal. For human subjects a “normal” body temperature is about 37.0° C. A grade 1 fever is an elevation in body temperature to about 38.0° C. to about 39.0° C. in a human subject, a grade 2 fever is an elevation in body temperature of greater than about 39.0° C. to about 40.0° C. in a human subject, a grade 3 fever is an elevation in body temperature greater than about 40.0° C. for less than 24 hours in a human subject, and a grade 4 fever is an elevation in body temperature greater than about 40.0° C. for more than 24 hours in a human subject.

As used herein, the term “chills” refers to a symptom characterized by a sensation of cold experienced by a subject. Grade 1 chills can include a mild sensation of cold with shivering and chattering of teeth, grade 2 chills can include moderate tremor of the entire body, controllable by narcotics, and grade 3 chills can include a severe or prolonged sensation of cold with tremors of the entire body, that is not responsive to narcotics.

As used herein, the term “symptom” includes both “signs” and “symptoms,” and refers to a departure from a normal function or feeling in a subject which is either apparent to a subject (symptom) or can be measured or observed in a subject (sign). Exemplary symptoms according to this disclosure include, without limitation, fever, chills, rash, pain, fatigue, nausea, C-reactive protein (CRP) elevation, or cytokine release.

As used herein, the term “healthcare provider” refers to individuals or institutions that directly interact and/or administer therapies to living subjects, e.g., human subjects. Non-limiting examples of healthcare providers include doctors, nurses, technicians, therapists, pharmacists, counselors, alternative medicine practitioners, medical facilities, doctor's offices, hospitals, emergency rooms, clinics, urgent care centers, alternative medicine clinics/facilities, and any other entity providing general and/or specialized treatment, assessment, maintenance, therapy, medication, and/or advice relating to all, or any portion of, a patient's state of health, including but not limited to general medical, specialized medical, surgical, and/or any other type of treatment, assessment, maintenance, therapy, medication and/or advice.

As used herein, the term “clinical laboratory” refers to a facility for obtaining data and/or the examination, and/or the processing of data obtained from a living subject and/or materials derived from a living subject, e.g., a human subject. Non-limiting examples of processing include radiographic (e.g., X-rays), fluorographic, tomographic (e.g., Positron Emission Tomography or PET-scans), or magnetic resonance (MRI) imaging of a subject, biological, biochemical, serological, chemical, immunohematological, hematological, biophysical, cytological, pathological, genetic, or other examination of materials derived from the human body, for the purpose of providing data or information, e.g., for the diagnosis, prevention, or treatment of any disease or impairment of, or the assessment of the health of living subjects, e.g., humans. These examinations can include procedures to obtain imaging data of the subject, to collect or otherwise obtain a sample, prepare, determine, measure, or otherwise describe the presence or absence of various substances in the body of a living subject, e.g., a human subject, or a sample obtained from the body of a living subject, e.g., a human subject.

As used herein, the term “healthcare benefits provider” encompasses individual parties, organizations, or groups providing, presenting, offering, paying for in whole or in part, or being otherwise associated with giving a patient access to one or more healthcare benefits, benefit plans, health insurance, and/or healthcare expense account programs.

In some embodiments, a healthcare provider can administer or instruct another healthcare provider to administer a therapy to treat a particular disease, disorder, or injury. A healthcare provider can implement or instruct another healthcare provider or patient, both under the first healthcare provider's control, to perform the following actions: submit to an imaging study or perform an imaging study on a patient, obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, administer a therapy (e.g., comparing baseline imaging results with results obtained following a treatment regimen), commence the administration of a therapy, cease the administration of a therapy, continue the administration of a therapy, temporarily interrupt the administration of a therapy, increase the amount of an administered therapeutic agent, decrease the amount of an administered therapeutic agent, continue the administration of an amount of a therapeutic agent, increase the frequency of administration of a therapeutic agent, decrease the frequency of administration of a therapeutic agent, maintain the same dosing frequency on a therapeutic agent, replace a therapy or therapeutic agent by at least another therapy or therapeutic agent, combine a therapy or therapeutic agent with at least another therapy or additional therapeutic agent.

In some aspects, a healthcare benefits provider can authorize or deny, for example, imaging studies, collection of a sample, processing of a sample, submission of a sample, receipt of a sample, transfer of a sample, analysis or measurement a sample, quantification a sample, provision of results obtained after analyzing/measuring/quantifying a sample, transfer of results obtained after analyzing/measuring/quantifying a sample, comparison/scoring of results obtained after analyzing/measuring/quantifying one or more samples, transfer of the comparison/score from one or more samples, administration of a therapy or therapeutic agent, commencement of the administration of a therapy or therapeutic agent, cessation of the administration of a therapy or therapeutic agent, continuation of the administration of a therapy or therapeutic agent, temporary interruption of the administration of a therapy or therapeutic agent, increase of the amount of administered therapeutic agent, decrease of the amount of administered therapeutic agent, continuation of the administration of an amount of a therapeutic agent, increase in the frequency of administration of a therapeutic agent, decrease in the frequency of administration of a therapeutic agent, maintain the same dosing frequency on a therapeutic agent, replace a therapy or therapeutic agent by at least another therapy or therapeutic agent, or combine a therapy or therapeutic agent with at least another therapy or additional therapeutic agent. In certain embodiments, a healthcare benefits provider can authorize or deny treatment based on the results of a companion diagnostic assay, e.g., imaging studies that show whether a certain therapy is effective in a given individual patient.

In some embodiments, a clinical laboratory can, for example, perform imaging studies on a patient under orders from a healthcare provider, compare baseline and follow-on imaging studies after a given therapy is administered, collect or obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, or other related activities. A clinical laboratory typically performs tests ordered by a healthcare provider or a healthcare benefits provider, and typically works under the healthcare provider's and/or healthcare benefits provider's control, or in a joint enterprise with healthcare provider and/or healthcare benefits provider.

### Methods of Treating Cancer

Provided herein is a method of treating cancer in a subject, the method comprising administering to the subject an effector cell engaging molecule, such as an effector cell engaging molecule disclosed herein. In some embodiments, the effector cell engaging molecule comprises one or more tumor targeting moieties and one or more effector cell engaging moieties, where the cumulative potency of the one or more tumor targeting moieties is greater than the cumulative potency of the one or more effector cell engaging moieties. In some embodiments, the effector cell engaging molecule comprises one or more tumor targeting moieties and one or more effector cell engaging moieties, where the total avidity of the one or more tumor targeting moieties is greater than the total avidity of the one or more effector cell engaging moieties. In some embodiments, the effector cell engaging molecule comprises tumor targeting moieties and one or more effector cell engaging moieties, where the effector cell engaging molecule is a multimeric binding molecule comprising two to five bivalent binding units or variants or fragments thereof and a J-chain or functional fragment or variant thereof, where each binding unit comprises two IgA or IgM heavy chain constant regions or multimerizing fragments or variants thereof, each associated with one of the tumor targeting moieties or a subunit thereof.

In some embodiments, the method comprises administering to the subject a therapeutically effective amount of an effector cell engaging molecule as provided herein. By “therapeutically effective dose or amount” or “effective amount” is intended an amount of a multimeric binding molecule that when administered brings about a positive therapeutic response with respect to treatment of subject.

Effective doses of compositions for treatment of a disease or disorder vary depending upon many different factors, including means of administration, target site, physiological state of the subject, whether the subject is human or an animal, other medications administered, and whether treatment is prophylactic or therapeutic. Usually, the subject is a human, but non-human mammals including transgenic mammals can also be treated.

In some embodiments, the method comprises (a) administering to the subject an effector cell engaging molecule at a determined dosage; and (b) monitoring the subject for an administration-related symptom. In some embodiments, the method comprises first administering to the subject an effector cell engaging molecule at a determined dosage; and second monitoring the subject for an administration-related symptom. In some embodiments, the method further comprises (c)(i) administering the effector cell engaging molecule to the subject at the same or a reduced dosage relative to the determined dosage if the subject had the administration-related symptom. In some embodiments, the method further comprises (c)(i) administering the effector cell engaging molecule to the subject at the determined dosage if the subject had the administration-related symptom. In some embodiments, the method further comprises (c)(ii) administering the effector cell engaging molecule to the subject at an increased dosage relative to the determined dosage if the subject did not have the administration-related symptom. In some embodiments, the method further comprises (d) repeating step (b) and (c). In some embodiments, steps (b) and (c) are repeated 1, 2, 3, 4, 5, 6, 7, 8, 9, or 10 times.

In some embodiments, the step (a) and the step (c)(i) or (c)(ii) administrations of the effector cell engaging molecule are 3 days to 2 weeks apart such as 3 days to 10 days, 3 days to 7 days, 3 days to 5 days, 5 days to 14 days, 5 days to 10 days, 5 days to 7 days, 7 days to 14 days, 7 days to 10 days, 3 days, 5 days, 7 days, 10 days, or 14 days apart.

In some embodiments, the determined dosage of the effector cell engaging molecule is 30 to 1000 mg, such as 30 to 50 mg, 30 to 100 mg, 30 to 200 mg, 30 to 500 mg, 50 to 100 mg, 50 to 200 mg, 50 to 500 mg, 50 to 750 mg, 50 to 1000 mg, 100 to 200 mg, 100 to 500 mg, 100 to 750 mg, 100 to 1000 mg, 200 mg to 500 mg, 200 to 750 mg, 200 to 1000 mg, 500 to 750 mg, 500 to 1000 mg, or 750 mg to 1000 mg. In some embodiments, the determined dosage of step (a) of the effector cell engaging molecule is 30 to 1000 mg, such as 30 to 50 mg, 30 to 100 mg, 30 to 200 mg, 30 to 500 mg, 50 to 100 mg, 50 to 200 mg, 50 to 500 mg, 50 to 750 mg, 50 to 1000 mg, 100 to 200 mg, 100 to 500 mg, 100 to 750 mg, 100 to 1000 mg, 200 mg to 500 mg, 200 to 750 mg, 200 to 1000 mg, 500 to 750 mg, 500 to 1000 mg, or 750 mg to 1000 mg. In some embodiments, the determined dosage of the effector cell engaging molecule is 30 mg, 50 mg, 100 mg, 200 mg, 500 mg, 750 mg, or 1000 mg. In some embodiments, the determined dosage of step (a) of the effector cell engaging molecule is 30 mg, 50 mg, 100 mg, 200 mg, 500 mg, 750 mg, or 1000 mg.

In some embodiments, the increased dosage of the effector cell engaging molecule in step (c)(ii) is 25%-1000% greater than the determined dosage of the effector cell engaging molecule, such as 25%-900%, 25%-800%, 25%-700%, 25%-600%, 25%-500%, 25%-400%, 25%-300%, 25%-200%, 25%-150%, 25%-100%, 25%-75%, 25%-50%, 50%-1000%, 50%-900%, 50%-800%, 50%-700%, 50%-600%, 50%-500%, 50%-400%, 50%-300%, 50%-200%, 50%-150%, 50%-100%, 50%-75%, 75%-1000%, 75%-900%, 75%-800%, 75-700%, 75%-600%, 75%-500%, 75%-400%, 75%-300%, 75%-200%, 75%-150%, 75%-100%, 100%-1000%, 100%-900%, 100%-800%, 100-700%, 100%-600%, 100%-500%, 100%-400%, 100%-300%, 100%-200%, 100%-150%, 150%-1000%, 150%-900%, 150%-800%, 150%-700%, 150%-600%, 150%-500%, 150%-400%, 150%-300%, 150%-200%, 200%-1000%, 200%-900%, 200%-800%, 200%-700%, 200%-600%, 200%-500%, 200%-400%, 200%-300%, 300%-1000%, 300%-900%, 300%-800%, 300%-700%, 300%-600%, 300%-500%, 300%-400%, 400%-1000%, 400%-900%, 400%-800%, 400%-700%, 400%-600%, 400%-500%, 500%-1000%, 500%-900%, 500%-800%, 500%-700%, 500%-600%, 600%-1000%, 600%-900%, 600%-800%, 600%-700%, 700%-1000%, 700%-900%, 700%-800%, 800%-1000%, 800%-900%, or 900%-1000% greater than the determined dosage of the effector cell engaging molecule.

Also provided herein is a method of treating cancer in a subject in need thereof, comprising: (a) administering a dosage of 10 mg to 50 mg of a T cell engaging molecule to the subject; (b) administering a dosage of 75 mg to 600 mg of the T cell engaging molecule to the subject at least 5 days after the administration of step (a), where the T cell engaging molecule is a multimeric binding molecule comprising five bivalent binding units and a modified J-chain, and where each binding unit comprises two IgM heavy chains, each comprising a heavy chain variable region (VH) and an IgM constant region and two light chains, each comprising a light chain variable region (VL) and a light chain constant region, where an associated VH and VL specifically bind to CD20 or a subunit thereof, where the VH comprises three immunoglobulin complementarity determining regions: HCDR1, HCDR2, and HCDR3, and the VL comprises three immunoglobulin complementarity determining regions: LCDR1, LCDR2, and LCDR3, where the HCDR1, HCDR2, HCDR3, LCDR1, LCDR2, and LCDR3 comprise, respectively, the amino acid sequence of SEQ ID NO: 55; SEQ ID NO: 56; SEQ ID NO: 57; SEQ ID NO: 58; SEQ ID NO: 59; and SEQ ID NO: 60, where the modified J-chain comprises a J-chain or functional fragment or variant thereof and an scFv molecule that specifically binds to CD3, and where the scFv comprises the amino acid sequence of SEQ ID NO: 31.

In some embodiments, the method further comprises (c) administering the dosage of the effector cell engaging molecule administered to the subject in step (b). In some embodiments, the method further comprises (a′) administering a dosage of the effector cell engaging molecule the same or greater than step (a) and lower than step (b), where step (a′) is after step (a) and before step (b). In some embodiments, the method further comprises (a″) administering a dosage of the effector cell engaging molecule the same or greater than step (a′) and lower than step (b), where step (a″) is after step (a′) and before step (b). In some embodiments, step (c) is performed 5 days to 8 weeks after step (b), such as 1 week to 3 weeks, 1 week, 2 weeks, or 3 weeks. In some embodiments, step (c) is performed 5 days to 8 weeks after step (a′), such as 1 week to 3 weeks, 1 week, 2 weeks, or 3 weeks. In some embodiments, step (c) is performed 5 days to 8 weeks after step (a″), such as 1 week to 3 weeks, 1 week, 2 weeks, or 3 weeks. In some embodiments, step (a′) is performed 5 days to 8 weeks after step (a), such as 1 week to 3 weeks, 1 week, 2 weeks, or 3 weeks. In some embodiments, step (a″) is performed 5 days to 8 weeks after step (a″), such as 1 week to 3 weeks, 1 week, 2 weeks, or 3 weeks.

Also provided herein is a method of treating cancer in a subject in need thereof, comprising administering a dosage of 75 mg to 600 mg of the T cell engaging molecule to the subject, where the T cell engaging molecule is a multimeric binding molecule comprising five bivalent binding units and a modified J-chain, where each binding unit comprises two IgM heavy chains, each comprising a heavy chain variable region (VH) and an IgM constant region and two light chains, each comprising a light chain variable region (VL) and a light chain constant region, where an associated VH and VL specifically bind to CD20 or a subunit thereof, where the VH comprises three immunoglobulin complementarity determining regions: HCDR1, HCDR2, and HCDR3, and the VL comprises three immunoglobulin complementarity determining regions: LCDR1, LCDR2, and LCDR3, where the HCDR1, HCDR2, HCDR3, LCDR1, LCDR2, and LCDR3 comprise, respectively, the amino acid sequence of SEQ ID NO: 55; SEQ ID NO: 56; SEQ ID NO: 57; SEQ ID NO: 58; SEQ ID NO: 59; and SEQ ID NO: 60, where the modified J-chain comprises a J-chain or functional fragment or variant thereof and an scFv molecule that specifically binds to CD3, and where the scFv comprises the amino acid sequence of SEQ ID NO: 31. In some embodiments, the subject had received a prior dosage of the T cell engaging molecule. In some embodiments, the prior dosage was lower than the dosage. In some embodiments, the prior dosage comprises two or three prior dosages. In some embodiments, the prior dosage comprises more than three prior dosages. In some embodiments, the first prior dosage was lower than the second prior dosage.

In some embodiments, a method provided herein comprises a series of dosages, such as a determined dosage and an increased dosage, a dosage and previously administered doses, or a method comprising administering different dosages in ordered steps. Exemplary, non-limiting dosage series are shown in Table 2.

TABLE 2
Exemplary Dosage Series (mg)

| Exemplary Series #1 | 10 to 50 | 75 to 600 | | |
|---|---|---|---|---|
| Exemplary Series #2 | 10 to 50 | 100 to 300 | | |
| Exemplary Series #3 | 10 to 50 | 100 | | |
| Exemplary Series #4 | 10 to 50 | 300 | | |
| Exemplary Series #5 | 10 to 50 | 50 to 100 | | |
| Exemplary Series #6 | 10 to 30 | 100 to 300 | | |
| Exemplary Series #7 | 10 to 30 | 100 | | |
| Exemplary Series #8 | 10 to 30 | 300 | | |
| Exemplary Series #9 | 10 to 25 | 100 to 300 | | |
| Exemplary Series #10 | 10 to 25 | 35 to 75 | 75 to 200 | |
| Exemplary Series #11 | 10 to 25 | 35 to 75 | 100 | |
| Exemplary Series #12 | 25 to 75 | 25 to 75 | 75 to 200 | |
| Exemplary Series #13 | 25 to 75 | 75 to 150 | 150 to 250 | |
| Exemplary Series #14 | 25 to 75 | 75 to 150 | 150 to 300 | |
| Exemplary Series #15 | 15 | 30 to 50 | 100 | |
| Exemplary Series #16 | 15 | 50 to 100 | 300 | |
| Exemplary Series #17 | 15 | 50 | 100 | |
| Exemplary Series #18 | 50 | 50 | 100 | |
| Exemplary Series #19 | 50 | 100 | 200 | |
| Exemplary Series #20 | 50 | 100 | 300 | |
| Exemplary Series #21 | 10 to 25 | 25 to 45 | 45 to 65 | 75 to 200 |
| Exemplary Series #22 | 10 to 25 | 25 to 45 | 45 to 65 | 100 |
| Exemplary Series #23 | 10 to 25 | 35 to 75 | 75 to 200 | 200 to 300 |
| Exemplary Series #24 | 25 to 75 | 75 to 150 | 150 to 250 | 250 to 300 |
| Exemplary Series #25 | 25 to 75 | 75 to 150 | 150 to 250 | 300 |
| Exemplary Series #26 | 15 | 30 | 50 | 100 |
| Exemplary Series #27 | 15 | 50 | 100 | 300 |
| Exemplary Series #28 | 50 | 100 | 200 | 300 |

In some embodiments, the next dosage in the series is administered 5 days to 3 weeks after the previous dosage. In some embodiments, the listed doses are each space 5 days to 3 weeks apart, such as 1 week apart or 3 weeks apart. In some embodiments, a dosage in the series is repeated. In some embodiments, the dosage is the series is repeated due to a symptom, such as a symptom described herein. In some embodiments, the last dosage listed in the series is repeated 1, 2, 3, 4, 5, 6, or more times. In some embodiments, the repeated last dosage is administered every three weeks and the previous dosages were administered every one week.

In some embodiments, the administering in steps, e.g., steps (a) and (c) of some embodiments, are carried out by a healthcare provider, e.g., a physician or personnel working under a physician's direction.

In some embodiments, monitoring of symptoms, e.g., in step (b) of some embodiments, is carried out by a healthcare provider, e.g., a physician or personnel working under a physician's direction. In some embodiments, the administration-related symptom comprises hypotension, chills, fever, elevated C reactive protein (CRP) level, fatigue, hypophosphatemia, anaemia, nausea, vomiting, insomnia, back pain, cytokine release, or a combination thereof. In some embodiments, the administration-related symptom comprises chills, fever, elevated C reactive protein (CRP) level, or a combination thereof. In some embodiments, elevated CRP level is defined as at or above 50 mg/dL CRP, such as at or above 60, 65, 70, 75, 80, 85, 90, 95, or 100 mg/dL.

In some embodiments, the method further comprises administering dexamethasone. In some embodiments, one or more administering steps further comprises administering dexamethasone. In some embodiments, the method comprises administering 10-30 mg of dexamethasone, such as 10 mg, 15 mg, 20 mg, 25 mg, or 30 mg of dexamethasone. In some embodiments, step (a) further comprises administering dexamethasone. In some embodiments, step (c) further comprises administering dexamethasone. In some embodiments, step (c) further comprises administering no dexamethasone. In some embodiments, step (c)(i) further comprises administering dexamethasone. In some embodiments, step (c)(i) further comprises administering no dexamethasone. In some embodiments, step (c)(ii) further comprises administering dexamethasone. In some embodiments, step (c)(ii) further comprises administering no dexamethasone. In some embodiments, the method further comprises administering dexamethasone. In some embodiments, step (a) further comprises administering dexamethasone, and step (c) further comprises administering dexamethasone. In some embodiments, step (a) further comprises administering dexamethasone, and step (c) further comprises administering no dexamethasone. In some embodiments, step (a) further comprises administering dexamethasone and step (c) further comprises administering a higher dosage of dexamethasone. In some embodiments, step (a) further comprises administering dexamethasone and step (c) further comprises administering lower dosage of dexamethasone.

In some embodiments, step (a) further comprises administering dexamethasone, and step (c)(i) further comprises administering dexamethasone. In some embodiments, step (a) further comprises administering dexamethasone, and step (c)(i) further comprises administering no dexamethasone. In some embodiments, step (a) further comprises administering dexamethasone and step (c)(i) further comprises administering a higher dosage of dexamethasone. In some embodiments, step (a) further comprises administering dexamethasone and step (c)(i) further comprises administering a lower dosage of dexamethasone.

In some embodiments, step (a) further comprises administering dexamethasone, and step (c)(ii) further comprises administering dexamethasone. In some embodiments, step (a) further comprises administering dexamethasone, and step (c)(ii) further comprises administering no dexamethasone. In some embodiments, step (a) further comprises administering dexamethasone and step (c)(ii) further comprises administering a higher dosage of dexamethasone. In some embodiments, step (a) further comprises administering dexamethasone and step (c)(ii) further comprises administering a lower dosage of dexamethasone.

In some embodiments, step (a) further comprises administering dexamethasone, step (c)(i) further comprises administering dexamethasone, and step (c)(ii) further comprises administering no dexamethasone. In some embodiments, step (a) further comprises administering dexamethasone, step (c)(i) further comprises administering no dexamethasone, and step (c)(ii) further comprises administering dexamethasone. In some embodiments, step (a) further comprises administering dexamethasone, step (c)(i) further comprises administering a higher dosage of dexamethasone, and step (c)(ii) further comprises administering no dexamethasone. In some embodiments, step (a) further comprises administering dexamethasone, step (c)(i) further comprises administering a higher dosage of dexamethasone, and step (c)(ii) further comprises administering a lower dosage of dexamethasone. In some embodiments, step (a) further comprises administering dexamethasone, step (c)(i) further comprises administering a lower dosage of dexamethasone, and step (c)(ii) further comprises administering no dexamethasone. In some embodiments, step (a) further comprises administering dexamethasone, step (c)(i) further comprises administering a lower dosage of dexamethasone, and step (c)(ii) further comprises administering a higher dosage of dexamethasone. In some embodiments, the subject is human.

In some embodiments, the subject did not receive any prior cancer therapies. In some embodiments, the subject had previously received a different cancer therapy. In some embodiments, the subject was resistant and/or refractory to the different cancer therapy. In some embodiments, the different cancer therapy comprises chemotherapy, radiation therapy, or immunotherapy, where the immunotherapy is different from the effector cell engaging molecule. In some embodiments, the immunotherapy is an anti-CD20 antibody. In some embodiments, the immunotherapy is rituximab. In some embodiments, the immunotherapy is an anti-CD20 and anti-CD3 multispecific antibody. In some embodiments, the immunotherapy is chimeric antigen receptor (CAR)-expressing cells, such as any CAR-expressing cells described herein.

In some embodiments, a method provided herein comprises administering rituximab. For example, in some embodiments, a method of treating cancer in a subject in need thereof comprises administering a T cell engaging molecule to the subject and administering rituximab to the subject.

In some embodiments, the subject previously received rituximab. For example, in some embodiments, a method of treating cancer in a subject in need thereof comprises administering a T cell engaging molecule to the subject, where the subject had previously been administered rituximab.

In some embodiments, the administering of the T cell engaging molecule occurs at least 1 week, at least 2 weeks, at least 1 month, at least 2 months, at least 3 months, at least 6 months, at least 9 months, at least 1 year, or at least 2 years after the rituximab was administered. In some embodiments, the administering of the T cell engaging molecule occurs less than 2 years, e.g., less than 1 year, less than 9 months, less than 6 months, less than 3 months, less than two months, or less than 1 month after the rituximab was administered. In some embodiments, the administering of the T cell engaging molecule occurs 1 week to 2 years after the rituximab was administered, such as 2 weeks to 1 year, 1 month to 1 year, 2 months to 1 year, 3 months to 1 year, 6 months to 1 year, or 9 months to 1 year.

In some embodiments, the subject has rituximab serum levels of 0 μg/mL to 200 μg/mL when the administering of the T cell engager occurs, such as 0 μg/mL to 100 μg/mL, 0 μg/mL to 70 μg/mL, 0 μg/mL to 67 μg/mL, 0 μg/mL to 25 μg/mL, 0 μg/mL to 23 μg/mL, 0 μg/mL to 7.5 μg/mL, 0 μg/mL to 2.5 μg/mL, 2.4 μg/mL to 200 μg/mL, 2.4 μg/mL to 100 μg/mL, 2.4 μg/mL to 70 μg/mL, 2.4 μg/mL to 67 μg/mL, 2.4 μg/mL to 25 μg/mL, 2.4 μg/mL to 23 μg/mL, 2.4 μg/mL to 7.5 μg/mL, 7.4 μg/mL to 200 μg/mL, 7.4 μg/mL to 100 μg/mL, 7.4 μg/mL to 70 μg/mL, 7.4 μg/mL to 67 μg/mL, 7.4 μg/mL to 25 μg/mL, or 7.4 μg/mL to 23 μg/mL.

In some embodiments, the rituximab administration comprises administering a dose of 375 mg/m² of rituximab. In some embodiments, the rituximab administration comprises administering rituximab once weekly for 4 to 8 weeks, such as for 4 weeks or for 8 weeks.

In some embodiments, the method of treating cancer further comprises administering CAR-expressing cells to the subject. In some embodiments, the method comprises administering CAR-expressing cells to the subject; and administering an effector cell engaging molecule to the subject. In some embodiments, the method comprises administering the CAR-expressing cells prior to administering the effector cell engaging molecule. In some embodiments, the method comprises administering the effector cell engaging molecule prior to administering the CAR-expressing cells. In some embodiments, the method comprises administering CAR-expressing cells to the subject, where the subject has previously been administered an effector cell engaging molecule. In some embodiments, the method comprises administering an effector cell engaging molecule to the subject, where the subject has previously been administered CAR-expressing cells.

In some embodiments, the CAR-expressing cells in the subject are below the level of detection prior to administering the effector cell engaging molecule. In some embodiments, the administering of the effector cell engaging molecule occurs at least 1 week, at least 2 weeks, at least 1 month, at least 2 months, at least 3 months, at least 6 months, at least 1 year, at least 2 years after the CAR-expressing cells were administered.

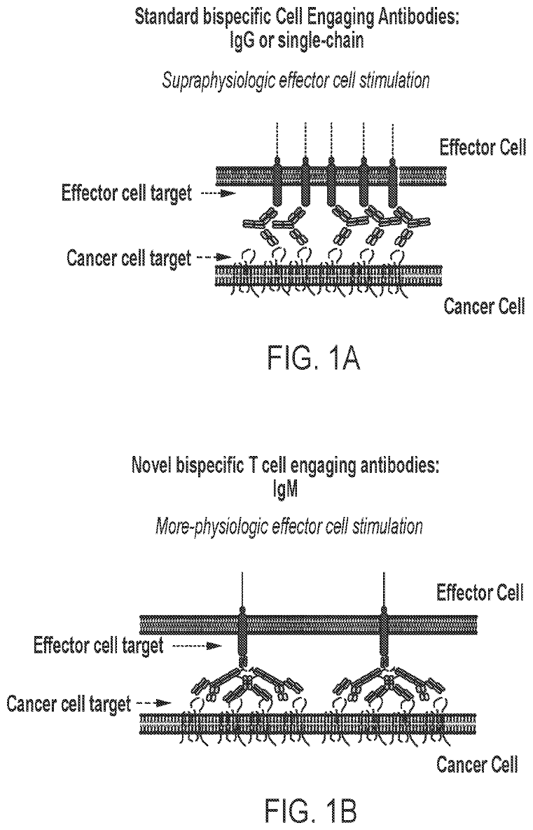

In certain embodiments, administration of an effector cell engaging molecule as provided herein to a subject stimulates effector cells at a more physiologic level of effector cell engagement than administration of an equivalent amount of a reference effector cell engaging molecule. An example of how an exemplary effector cell engaging molecules can generate a more physiologic level of effector cell engagement compared to a standard bispecific cell engaging antibody is depicted in FIGS. 1A-1B. By “an equivalent amount” is meant, e.g., an amount measured by molecular weight, e.g., in total milligrams, or alternatively, a molar equivalent, e.g., where equivalent numbers of molecules are administered. In certain embodiments the effector cell engaging molecule as provided herein comprises five, six, seven, eight, nine, ten, or eleven antigen-binding domains, where the sum total of tumor-specific binding domains is greater than the sum total of effector cell binding domain. In certain embodiments the effector cell engaging molecule as provided herein is IGM-2323. In certain embodiments the reference effector cell engaging molecule comprises exactly two antigen binding domains, where one antigen binding domain specifically binds to a tumor antigen, e.g., CD20, and one binding domain binds to an effector cell, e.g., a T cell. In certain embodiments the reference effector cell engaging molecule is mosunetuzumab (U.S. Pat. No. 10,174,1124) or odronextamab (U.S. Pat. No. 9,657,102).

In some embodiments, the reference effector cell engaging molecule comprises one or more reference tumor targeting moieties and one or more reference effector cell engaging moieties, where the cumulative potency of the one or more reference tumor targeting moieties is not greater than the cumulative potency of the one or more reference effector cell engaging moieties, and where the one or more reference tumor targeting moieties and the one or more tumor targeting moieties bind to the same tumor target and the one or more reference effector cell engaging moieties and the one or more effector cell engaging moieties bind to the same effector cell engaging target. In some embodiments, reference effector cell engaging molecule comprises one or more reference tumor targeting moieties and one or more reference effector cell engaging moieties, where the total avidity of the one or more reference tumor targeting moieties is not greater than the total avidity of the one or more reference effector cell engaging moieties, and where the one or more reference tumor targeting moieties and the one or more tumor targeting moieties bind to the same tumor target and the one or more reference effector cell engaging moieties and the one or more effector cell engaging moieties bind to the same effector cell engaging target. In certain embodiments, the reference effector cell engaging molecule is a monomeric binding polypeptide, such as an IgG, comprising one reference tumor targeting moiety and one reference effector cell engaging moiety, and where the reference tumor targeting moiety and the one or more tumor targeting moieties bind to the same tumor target and the reference effector cell engaging moieties and the one or more effector cell engaging moieties bind to the same effector cell engaging target. In some embodiments, the one or more (e.g., one) reference tumor targeting moieties and the one or more tumor targeting moieties comprise the same binding motifs. In some embodiments, the one or more (e.g., one) reference tumor targeting moieties and the one or more tumor targeting moieties comprise the same binding motifs.

In certain embodiments, administration of an effector cell engaging molecule as disclosed herein results in a greater release of interferon-γ (IFNγ) than administration of an equivalent amount of a reference effector cell engaging molecule. In some embodiments, the method comprises administering the effector cell engaging molecule two or more times, and the 2^{nd}, 3^{rd}, 4^{th}, or 5^{th} etc. administration results in a greater release of IFNγ than the 2^{nd}, 3^{rd}, 4^{th}, or 5^{th} etc. administration of an equivalent amount of a reference effector cell engaging molecule described herein.

In certain embodiments, administration of an effector cell engaging molecule as disclosed herein results in a lesser release of interleukin-6 (IL-6) than administration of an equivalent amount of a reference effector cell engaging molecule. In some embodiments, the method comprises administering the effector cell engaging molecule two or more times, and the 2^{nd}, 3^{rd}, 4^{th}, or 5^{th} etc. administration results in a lesser release of IL-6 than the 2^{nd}, 3^{rd}, 4^{th}, or 5^{th} etc. administration of an equivalent amount of a reference effector cell engaging molecule described herein.

The subject can be any animal, e.g., a mammal, in need of treatment or prevention, in certain embodiments, the subject is a human subject.

In its simplest form, a preparation to be administered to a subject is an effector cell engaging molecule as described herein administered in a conventional dosage form, which can be combined with a pharmaceutical excipient, carrier or diluent as described elsewhere herein, such as in a composition described herein.

An effector cell engaging molecule as described herein can be administered by any suitable method, e.g., parenterally, intraventricularly, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

Also provided herein is a method of preparing a subject for administration of chimeric antigen receptor (CAR)-expressing cells, such as any CAR-expressing cells disclosed herein, the method comprising administering to the subject an effector cell engaging molecule as provided herein, where the administration occurs prior to the administration of the CAR-expressing cells. In some embodiments, the method further comprises administering the CAR-expressing cells to the subject.

Also provided herein is a method of treating cancer in a subject in need thereof, comprising administering CAR-expressing cells, such as any CAR-expressing cells disclosed herein, to the subject, where the subject had previously been administered an effector cell engaging molecule as provided herein.

In some embodiments, the method of treating cancer, such as any method of treating cancer provided herein, is a method of treating a tumor antigen positive cancer, where the one or more tumor targeting moieties of the effector cell engaging molecule bind the tumor antigen. In some embodiments, the cancer is a CD20, CD38, CD123, tumor-associated calcium signal transducer 2 (TROP-2), mesothelin, glypican-3 (GPC-3), prostate-specific membrane antigen (PSMA), globo-H, chondroitin sulfate proteoglycan 4 (CSPG-4), or carcinoembryonic antigen (CEA) positive cancer. In some embodiments, the cancer is a CD20 positive cancer. In some embodiments, the method of treating cancer, such as any method of treating cancer provided herein, is a method of treating a leukemia, lymphoma, or myeloma, e.g., non-Hodgkin lymphoma (NHL), e.g., diffuse large B-cell lymphoma (DLBCL), follicular lymphoma (FL), mantle cell lymphoma (MCL), or marginal zone lymphoma (MZL). In some embodiments, the method of treating cancer, such as any method of treating a relapsed or refractory cancer, such as relapsed or refractory NHL.

### Pharmaceutical Compositions and Administration Methods

In certain embodiments, the molecules or cells administered in the methods disclosed herein are part of a composition, e.g., a pharmaceutical composition. A composition as provided herein can further include a pharmaceutically acceptable carrier and/or excipient and can be formulated so as to be suitable for a desired mode of administration.

Methods of preparing molecules or cells employed in the methods as provided herein can be determined by a skilled person in view of this disclosure. A suitable pharmaceutical composition can include a buffer (e.g., acetate, phosphate, or citrate buffer), and/or a surfactant (e.g., polysorbate), optionally a stabilizer agent (e.g., human albumin), etc.

As discussed herein, a molecule or cells, such as dexamethasone, CAR-expressing cells, or an effector engaging molecule, such as a multimeric binding molecule, as provided herein can be administered in a pharmaceutically effective amount for the treatment of a subject in need thereof. In this regard, it will be appreciated that the molecule or cells can be formulated so as to facilitate administration and promote stability of the active agent. Pharmaceutical compositions accordingly can include a pharmaceutically acceptable, non-toxic, sterile carrier such as physiological saline, non-toxic buffers, preservatives, and the like. A pharmaceutically effective amount of a multimeric binding molecule as provided herein means an amount sufficient to achieve effective binding to a target and to achieve a therapeutic benefit. Suitable formulations are described in Remington's Pharmaceutical Sciences (Mack Publishing Co.) 16th ed. (1980).

Certain pharmaceutical compositions provided herein can be orally administered in an acceptable dosage form including, e.g., capsules, tablets, aqueous suspensions, or solutions.

Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents. In some embodiments, the pharmaceutical composition is administered by nasal aerosol. In some embodiments, the pharmaceutical composition is for administration by nasal aerosol. In some embodiments, the pharmaceutical composition, such as a pharmaceutical composition for administration by nasal aerosol, comprises a pH adjuster, such as HCl; a buffer; an emulsifier, such as polysorbate or carbomer; sugar or mono- or polyol, such as a monosaccharide (e.g., glucose, dextrose, or fructose), disaccharide (e.g., sucrose, lactose, or maltose), ribose, glycerine, sorbitol, xylitol, inositol, propylene glycol, galactose, mannose, xylose, rhamnose, glutaraldehyde, ethanol, mannitol, polyethylene glycol, glycerol, chitosal, phenylethyl alcohol; a preservative; cellulose, such as microcrystalline cellulose or carboxymethylcellulose; or mixtures thereof.

In some embodiments, the pharmaceutical composition is administered by inhalation. In some embodiments, the pharmaceutical composition is for administration by inhalation. In some embodiments, the pharmaceutical composition, such as a pharmaceutical composition for administration by inhalation, is a dry powder, such as for a dry powder inhaler, or a liquid, such as for a nebulizer, such as an airjet-compressor nebulizer or a mesh-based nebulizer. In some embodiments, the pharmaceutical composition, such as a pharmaceutical composition for administration by inhalation, comprises sugar or mono- or polyol, such as lactose, trelose, mannitol, sorbitol; buffer, such as histidine, proline, or arginine buffer; saline; polysorbate; or mixtures thereof.

The amount of a molecule or cells that can be combined with carrier materials to produce a single dosage form will vary depending, e.g., upon the subject treated and the particular mode of administration. The composition can be administered as a single dose, multiple doses or over an established period of time in an infusion. Dosage regimens also can be adjusted to provide the optimum desired response (e.g., a therapeutic or prophylactic response).

In keeping with the scope of the present disclosure, a molecule or cells as described herein can be administered to a subject in need of therapy in an amount sufficient to produce a therapeutic effect. A molecule or cells as provided herein can be administered to the subject in a conventional dosage form prepared by combining the molecule or cells described herein with a conventional pharmaceutically acceptable carrier or diluent according to known techniques. The form and character of the pharmaceutically acceptable carrier or diluent can be dictated by the amount of active ingredient with which it is to be combined, the route of administration and other well-known variables.

This disclosure also provides for the use of a molecule or CAR-expressing cells as described herein in the manufacture of a medicament for treating cancer for administration according to a method provided herein. This disclosure also provides pharmaceutical compositions, such as any pharmaceutical composition described herein for use in a method described herein.

### Chimeric Antigen Receptors (CARs) and CAR-Expressing Cells

Methods of manufacturing and administering CAR-expressing cells are known in the art. See, e.g., Chandran, SS, and CA Klebanoff, Immunol. Rev. 290:127-147 (2019) and U.S. Pat. Nos. 7,446,190, 8,399,645, and 7,638,325.

In some embodiments, the CAR comprises an external targeting domain that binds CD19, BCMA, CD20, CD22, CD23, or ROR1. In some embodiments, the CAR expressing cells are CAR-T cells or CAR-NK cells. In some embodiments, the CAR expressing cells are CAR-T cells or CAR-NK cells. In some embodiments, the CAR comprises a CD3 signaling domain, a 4-1BB stimulatory domain, and/or CD28 stimulatory domain. In some embodiments, the CAR-T cells are lisocabtagene maraleucel, axicabtagene ciloleucel, tisagenlecleucel, brexucabtagene autoleucel, idecabtagene vicleucel, ciltacabtagene autoleucel, or orvacabtagene autoleucel. In some embodiments, the CAR-T cells are lisocabtagene maraleucel, axicabtagene ciloleucel, tisagenlecleucel, or brexucabtagene autoleucel.

### Effector Cell Engaging Molecules

In some embodiments, the effector cell engaging molecule comprises one or more tumor targeting moieties and one or more effector cell engaging moieties, where the cumulative potency of the one or more tumor targeting moieties is greater than the cumulative potency of the one or more effector cell engaging moieties. The cumulative potency can be affected by multiple factors, e.g., the number of binding moieties, the avidity or affinity of the binding moieties, the ability of binding moieties to reach target, e.g., whether there is steric hinderance. In some embodiments, a greater affinity of the tumor targeting moiety/moieties than the effector cell engaging moiety/moieties contributes to the greater cumulative potency of the one or more effector cell engaging moieties. In some embodiments, a greater avidity of the tumor targeting moiety/moieties than the effector cell engaging moiety/moieties contributes to the greater cumulative potency of the one or more effector cell engaging moieties. In some embodiments, a greater size of the tumor targeting moiety/moieties or the portion of the molecule adjacent to the tumor targeting moiety/moieties than the effector cell engaging moiety/moieties contributes to the greater cumulative potency of the one or more effector cell engaging moieties.

In some embodiments, the effector cell engaging molecule comprises one or more tumor targeting moieties and one or more effector cell engaging moieties, where the total avidity of the one or more tumor targeting moieties is greater than the total avidity of the one or more effector cell engaging moieties. In some embodiments, the ratio of the total avidity of the one or more tumor targeting moieties and the total avidity of the one or more effector cell engaging moieties (avidity T:E) is greater than a reference avidity ratio. In some embodiments, the reference avidity ratio is the ratio of the avidities of the tumor targeting and effector cell engaging moieties in mosunetuzumab or odronextamab. In some embodiments, the avidity T:E is at least 2-fold greater than the reference avidity ratio, such as four-fold, five-fold, or six-fold greater than the reference avidity ratio.

In some embodiments, the effector cell engaging molecule comprises one, two, four, eight, or ten tumor targeting moieties. In some embodiments, the effector cell engaging molecule comprises one or two effector cell engaging moieties. In some embodiments, the effector cell engaging molecule comprises only one effector cell engaging moiety. In some embodiments, the ratio of the tumor targeting moieties to the effector cell engaging moieties is 2:1, 4:1, 8:1, or 10:1. In some embodiments, the ratio of the tumor targeting moieties to the effector cell engaging moieties is 4:1 or 10:1. In some embodiments, the ratio of tumor targeting moieties to effector cell engaging moieties is 10:1.

In some embodiments, the effector cell is a T cell, e.g., a cytotoxic T cell (CTL) or a T helper cell, a plasma cell, or a natural killer (NK) cell. In certain embodiments, the effector cell is a T cell or a NK cell. In some embodiments, the effector cell is an NK cell and the antigen binding domain on the effector cell engaging molecule specifically binds to CD16. In some embodiments, the effector cell is a T cell and the antigen binding domain on the effector cell engaging molecule specifically binds to CD3.

In some embodiments, the one or more effector cell engaging moieties comprise an antibody or antigen-binding fragment thereof, such as a Fab, a Fab′, a F(ab′)2, a Fd, a Fv, a single-chain Fv (scFv), a disulfide-linked Fv (sdFv), or any combination thereof. In some embodiments, the one or more effector cell engaging moieties comprise a scFv.

In some embodiments, the one or more effector cell engaging moieties bind CD16 or CD3. In some embodiments, the one or more effector cell engaging moieties bind CD3. In some embodiments, the one or more effector cell engaging moieties comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 SEQ ID NO: 14 and SEQ ID NO: 15, SEQ ID NO: 16 and SEQ ID NO: 17, SEQ ID NO: 18 and SEQ ID NO: 19, SEQ ID NO: 20 and SEQ ID NO: 21, SEQ ID NO: 22 and SEQ ID NO: 23, SEQ ID NO: 24 and SEQ ID NO: 25, or SEQ ID NO: 26 and SEQ ID NO: 27, respectively with zero, one, or two single amino acid substitutions in one or more of the HCDRs or LCDRs. In some embodiments, the one or more effector cell engaging moieties comprise a heavy chain variable region (VH) and a light chain variable region (VL), where the VH and VL comprise amino acid sequences at least 80%, at least 85%, at least 90%, at least 95% or 100% identical to SEQ ID NO: 14 and SEQ ID NO: 15, SEQ ID NO: 16 and SEQ ID NO: 17, SEQ ID NO: 18 and SEQ ID NO: 19, SEQ ID NO: 20 and SEQ ID NO: 21, SEQ ID NO: 22 and SEQ ID NO: 23, SEQ ID NO: 24 and SEQ ID NO: 25, or SEQ ID NO: 26 and SEQ ID NO: 27, respectively. In some embodiments, the one or more effector cell engaging moieties comprise an scFv comprising an amino acid sequence at least 80%, at least 85%, at least 90%, at least 95% or 100% identical to SEQ ID NO: 31, SEQ ID NO: 35, SEQ ID NO: 39, SEQ ID NO: 43, SEQ ID NO: 47, or SEQ ID NO: 51.

The effector cell engaging molecules employed in the methods provided herein comprise one or more tumor targeting moieties that bind a tumor antigen. In certain embodiments, the tumor targeting molecules comprise a ligand that binds the tumor antigen, such as an antigen receptor, or an antibody or antigen-binding fragment thereof, such as a Fab, a Fab′, a F(ab′)2, a Fd, a Fv, a single-chain Fv (scFv), a disulfide-linked Fv (sdFv), or any combination thereof.

The tumor antigen can be any tumor target. In some embodiments, the tumor antigen is a tumor-specific antigen. In some embodiments, is a tumor-associated antigen. In some embodiments, the tumor antigen is a polypeptide, a nucleic acid, a carbohydrate, or a lipid. Exemplary tumor targets are disclosed in U.S. Pat. Nos. 10,400,038 and 10,618,978, which are incorporated herein in their entireties. In some embodiments, the tumor antigen is CD20, CD38, CD123, tumor-associated calcium signal transducer 2 (TROP-2), mesothelin, glypican-3 (GPC-3), prostate-specific membrane antigen (PSMA), globo-H, chondroitin sulfate proteoglycan 4 (CSPG-4), or carcinoembryonic antigen (CEA). In some embodiments, the tumor antigen is CD20.

### Multimeric Binding Molecules

Antibodies and antibody-like molecules that can multimerize, such as IgA and IgM antibodies, have emerged as promising drug candidates, e.g., in the fields of immuno-oncology and infectious diseases, allowing for improved specificity, improved avidity, and the ability to bind to multiple binding targets. See, e.g., U.S. Pat. Nos. 9,951,134, 9,938,347, and 10,400,038, U.S. Patent Application Publication Nos. US 2019-0100597, US 2018-0009897, US 2019-0330374, US 2019-0330360, US 2019-0338040, US 2019-0338041, US 2019-0185570, US 2018-0265596, US 2018-0118816, US 2018-0118814, and US 2019-0002566, and PCT Publication Nos. WO 2018/187702, WO 2019/165340, and WO 2019/169314, the contents of which are incorporated herein by reference in their entireties.

Provided herein are methods that employ effector cell engaging molecules that are multimeric binding molecules comprising two to five bivalent binding units or variants or fragments thereof, and a J-chain or functional fragment or variant thereof, where each binding unit comprises two IgA or IgM heavy chain constant regions or multimerizing fragments or variants thereof, each associated with one of the tumor targeting moieties or a subunit thereof.

In some embodiments, the multimeric binding molecules are dimeric and comprise two bivalent binding units or variants or fragments thereof. In some embodiments, the multimeric binding molecules are dimeric, comprise two bivalent binding units or variants or fragments thereof, where each binding unit comprises two IgA heavy chain constant regions or multimerizing fragments or variants thereof.

In some embodiments, the multimeric binding molecules are tetrameric and comprise four bivalent binding units or variants or fragments thereof. In some embodiments, the multimeric binding molecules are tetrameric, comprise four bivalent binding units or variants or fragments thereof, where each binding unit comprises two IgA heavy chain constant regions or multimerizing fragments or variants thereof.

In some embodiments, the multimeric binding molecules are pentameric and comprise five bivalent binding units or variants or fragments thereof. In some embodiments, the multimeric binding molecules are pentameric and comprise five bivalent binding units or variants or fragments thereof, where each binding unit comprises two IgM heavy chain constant regions or multimerizing fragments or variants thereof.

In certain embodiments, heavy chain constant regions in the provided binding molecules are each associated with a tumor targeting moiety or subunit thereof, such as a binding domain, e.g., a ligand or an antibody antigen-binding domain, e.g., a scFv, a VHH or the VH subunit of an antibody antigen-binding domain.

In certain embodiments, the binding domains are antibody-derived antigen-binding domains, e.g., a scFv associated with the heavy chain constant regions or a VH subunit of an antibody binding domain associated with the heavy chain constant regions.

In certain embodiments, each binding unit comprises two heavy chains each comprising a VH situated amino terminal to the heavy chain constant region, and two immunoglobulin light chains each comprising a light chain variable domain (VL) situated amino terminal to an immunoglobulin light chain constant region, e.g., a kappa or lambda constant region. The provided VH and VL combine to form a tumor targeting moiety that specifically binds to the target.

In certain embodiments, the tumor antigen is CD20 and the tumor targeting moieties comprise a heavy chain variable region (VH) and a light chain variable region (VL). In certain embodiments, the tumor targeting moieties comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 28 and SEQ ID NO: 29, with zero, one, or two single amino acid substitutions in one or more of the HCDRs or LCDRs.

In certain embodiments, the tumor targeting moieties comprise a heavy chain variable region (VH) and a light chain variable region (VL), where the VH and VL comprise amino acid sequences at least 80%, at least 85%, at least 90%, at least 95% or 100% identical to SEQ ID NO: 28 and SEQ ID NO: 29.

### IgM Antibodies, IgM-Like Antibodies, Other IgM-Derived Binding Molecules

IgM is the first immunoglobulin produced by B cells in response to stimulation by antigen. IgM is naturally present at around 1.5 mg/ml in serum with a half-life of about 5 days. IgM is a pentameric or hexameric molecule and thus includes five or six binding units. An IgM binding unit typically includes two light and two heavy chains. While an IgG heavy chain constant region contains three heavy chain constant domains (CH1, CH2 and CH3), the heavy (μ) constant region of IgM additionally contains a fourth constant domain (CH4) and includes a C-terminal “tailpiece.” The human IgM constant region typically comprises the amino acid sequence SEQ ID NO: 1 (identical to, e.g., GenBank Accession Nos. pir∥S37768, CAA47708.1, and CAA47714.1, allele IGHM*03) or SEQ ID NO: 2 (identical to, e.g., GenBank Accession No. sp|01871.4, allele IGHM*04). The human Cμ1 region ranges from about amino acid 5 to about amino acid 102 of SEQ ID NO: 1 or SEQ ID NO: 2; the human Cμ2 region ranges from about amino acid 114 to about amino acid 205 of SEQ ID NO: 1 or SEQ ID NO: 2, the human Cμ3 region ranges from about amino acid 224 to about amino acid 319 of SEQ ID NO: 1 or SEQ ID NO: 2, the Cμ4 region ranges from about amino acid 329 to about amino acid 430 of SEQ ID NO: 1 or SEQ ID NO: 2, and the tailpiece ranges from about amino acid 431 to about amino acid 453 of SEQ ID NO: 1 or SEQ ID NO: 2.

Other forms and alleles of the human IgM constant region with minor sequence variations exist, including, without limitation, GenBank Accession Nos. CAB37838.1, and pir∥MHHU. The amino acid substitutions, insertions, and/or deletions at positions corresponding to SEQ ID NO: 1 or SEQ ID NO: 2 described and claimed elsewhere in this disclosure can likewise be incorporated into alternate human IgM sequences, as well as into IgM constant region amino acid sequences of other species.

Each IgM heavy chain constant region can be associated with a binding domain, e.g., an antigen-binding domain, e.g., a scFv or VHH, or a subunit of an antigen-binding domain, e.g., a VH region. Exemplary antigen-binding domains are described elsewhere herein. In certain embodiments the binding domain can be a non-antibody binding domain.

Five IgM binding units can form a complex with an additional small polypeptide chain (the J-chain), or a functional fragment, variant, or derivative thereof, to form a pentameric IgM antibody or IgM-like antibody, as discussed elsewhere herein. The precursor form of the human J-chain is presented as SEQ ID NO: 6. The signal peptide extends from amino acid 1 to about amino acid 22 of SEQ ID NO: 6, and the mature human J-chain extends from about amino acid 23 to amino acid 159 of SEQ ID NO: 6. The mature human J-chain includes the amino acid sequence SEQ ID NO: 7.

Exemplary variant and modified J-chains are provided elsewhere herein. Without the J-chain, an IgM antibody or IgM-like antibody typically assembles into a hexamer, comprising up to twelve antigen-binding domains. With a J-chain, an IgM antibody or IgM-like antibody typically assembles into a pentamer, comprising up to ten antigen-binding domains, or more, if the J-chain is a modified J-chain comprising one or more heterologous polypeptides comprising additional antigen-binding domain(s). The assembly of five or six IgM binding units into a pentameric or hexameric IgM antibody or IgM-like antibody is thought to involve the Cμ4 and tailpiece domains. See, e.g., Braathen, R., et al., J. Biol. Chem. 277:42755-42762 (2002). Accordingly, a pentameric or hexameric IgM antibody typically includes at least the Cμ4 and tailpiece domains (also referred to herein collectively as Cμ4-tp). A “multimerizing fragment” of an IgM heavy chain constant region thus includes at least the Cμ4-tp domains. An IgM heavy chain constant region can additionally include a Cμ3 domain or a fragment thereof, a Cμ2 domain or a fragment thereof, a Cμ1 domain or a fragment thereof, and/or other IgM heavy chain domains. In certain embodiments, an IgM-derived binding molecule, e.g., an IgM antibody, IgM-like antibody, or other IgM-derived binding molecule as provided herein can include a complete IgM heavy (μ) chain constant domain, e.g., SEQ ID NO: 1 or SEQ ID NO: 2, or a variant, derivative, or analog thereof, e.g., as provided herein.

In certain embodiments, the disclosure provides a multimeric binding molecule, e.g., pentameric binding molecule, where the binding molecule includes ten IgM-derived heavy chains, and where the IgM-derived heavy chains comprise IgM heavy chain constant regions each associated with one of the tumor targeting moieties or a subunit thereof. In certain embodiments, the disclosure provides an IgM antibody, IgM-like antibody, or IgM-derived binding molecule that includes five bivalent binding units, where each binding unit includes two IgM or IgM-like heavy chain constant regions or multimerizing fragments or variants thereof, each associated with one of the tumor targeting moieties or a subunit thereof. In certain embodiments, the two IgM heavy chain constant regions included in each binding unit are human heavy chain constant regions. In some embodiments, the heavy chains are glycosylated. In some embodiments, the heavy chains can be mutated to affect glycosylation.

Where the IgM antibody, IgM-like antibody, or other IgM-derived binding molecule provided in this disclosure is pentameric, the IgM antibody, IgM-like antibody, or other IgM-derived binding molecule typically further include a J-chain, or functional fragment or variant thereof. In certain embodiments, the J-chain is a modified J-chain or variant thereof that further comprises one or more heterologous moieties attached to the J-chain, as described elsewhere herein. In certain embodiments, the J-chain can be mutated to affect, e.g., enhance, the serum half-life of the IgM antibody, IgM-like antibody, or other IgM-derived binding molecule provided herein, as discussed elsewhere in this disclosure. In certain embodiments the J-chain can be mutated to affect glycosylation, as discussed elsewhere in this disclosure.

An IgM heavy chain constant region can include one or more of a Cμ1 domain or fragment or variant thereof, a Cμ2 domain or fragment or variant thereof, a Cμ3 domain or fragment or variant thereof, and/or a Cμ4 domain or fragment or variant thereof, provided that the constant region can serve a desired function in the IgM antibody, IgM-like antibody, or other IgM-derived binding molecule, e.g., associate with second IgM constant region to form a binding unit with one, two, or more antigen-binding domain(s), and/or associate with other binding units (and in the case of a pentamer, a J-chain) to form a hexamer or a pentamer. In certain embodiments the two IgM heavy chain constant regions or fragments or variants thereof within an individual binding unit each comprise a Cμ4 domain or fragment or variant thereof, a tailpiece (tp) or fragment or variant thereof, or a combination of a Cμ4 domain and a TP or fragment or variant thereof. In certain embodiments the two IgM heavy chain constant regions or fragments or variants thereof within an individual binding unit each further comprise a Cμ3 domain or fragment or variant thereof, a Cμ2 domain or fragment or variant thereof, a Cμ1 domain or fragment or variant thereof, or any combination thereof.

In some embodiments, the binding units of the IgM antibody, IgM-like antibody, or other IgM-derived binding molecule comprise two light chains. In some embodiments, the binding units of the IgM antibody, IgM-like antibody, or other IgM-derived binding molecule comprise two fragments light chains. In some embodiments, the light chains are kappa light chains. In some embodiments, the light chains are lambda light chains. In some embodiments, each binding unit comprises two immunoglobulin light chains each comprising a VL situated amino terminal to an immunoglobulin light chain constant region.

### IgA Antibodies, IgA-Like Antibodies, Other IgA-Derived Binding Molecules

IgA plays a critical role in mucosal immunity and comprises about 15% of total immunoglobulin produced. IgA can be monomeric or multimeric, forming primarily dimeric molecules, but can also assemble as trimers, tetramers, and/or pentamers. See, e.g., de Sousa-Pereira, P., and J. M. Woof, Antibodies 8:57 (2019). An IgA binding unit includes two light and two heavy chains. IgA contains three heavy chain constant domains (Cα1, Cα2 and Cα3), a hinge region between Cα1 and Cα2, and includes a C-terminal “tailpiece.” Human IgA has two subtypes, IgA1 and IgA2. The human IgA1 constant region typically includes the amino acid sequence SEQ ID NO: 3. The human Cα1 domain extends from about amino acid 6 to about amino acid 98 of SEQ ID NO: 3; the human IgA1 hinge region extends from about amino acid 102 to about amino acid 124 of SEQ ID NO: 3, the human Cα3 domain extends from about amino acid 228 to about amino acid 330 of SEQ ID NO: 3, and the tailpiece extends from about amino acid 331 to about amino acid 352 of SEQ ID NO: 3. The human IgA2 constant region typically includes the amino acid sequence SEQ ID NO: 4. The human Cα1 domain extends from about amino acid 6 to about amino acid 98 of SEQ ID NO: 4; the human IgA2 hinge region extends from about amino acid 102 to about amino acid 111 of SEQ ID NO: 4, the human Ca2 domain extends from about amino acid 113 to about amino acid 206 of SEQ ID NO: 4, the human Ca3 domain extends from about amino acid 215 to about amino acid 317 of SEQ ID NO: 4, and the tailpiece extends from about amino acid 318 to about amino acid 340 of SEQ ID NO: 4.

Two IgA binding units can form a complex with two additional polypeptide chains, the J-chain (e.g., the mature human J-chain of SEQ ID NO: 7) and the secretory component (precursor, SEQ ID NO: 5, mature: amino acids 19 to 603 of SEQ ID NO: 5) to form a secretory IgA (sIgA) antibody. The assembly of IgA binding units into a dimeric sIgA antibody is thought to involve the Cα3 and tailpiece domains (also referred to herein collectively as the Caβ-tp domain). Accordingly, a dimeric sIgA antibody provided in this disclosure typically includes IgA constant regions that include at least the Cα3 and tailpiece domains. Four IgA binding unites can likewise form a tetramer complex with a J-chain. A sIgA antibody can also form as a higher order multimer, e.g., a tetramer.

An IgA heavy chain constant region can additionally include a Cα2 domain or a fragment thereof, an IgA hinge region, a Cα1 domain or a fragment thereof, and/or other IgA heavy chain domains. In certain aspects, an IgA antibody or IgA-like binding molecule as provided herein can include a complete IgA heavy (α) chain constant domain (e.g., SEQ ID NO: 3 or SEQ ID NO: 4), or a variant, derivative, or analog thereof. In some embodiments, the IgA heavy chain constant regions or multimerizing fragments thereof are human IgA constant regions.

In some embodiments, each binding unit of an IgA antibody, IgA-like antibody, or other IgA-derived binding molecule comprises two light chains. In some embodiments, each binding unit of an IgA antibody, IgA-like antibody, or other IgA-derived binding molecule comprises two fragments light chains. In some embodiments, the light chains are kappa light chains. In some embodiments, the light chains are lambda light chains. In some embodiments the light chains are chimeric kappa-lambda light chains. In some embodiments, each binding unit comprises two immunoglobulin light chains each comprising a VL situated amino terminal to an immunoglobulin light chain constant region.

### J-Chains and Functional Fragments or Variants Thereof

In certain embodiments, the multimeric binding molecule provided herein comprises a J-chain or functional fragment or variant thereof. In certain embodiments, the multimeric binding molecule provided herein is pentameric and comprises a J-chain or functional fragment or variant thereof. In certain embodiments, the multimeric binding molecule provided herein is a dimeric IgA molecule or a pentameric IgM molecule and comprises a J-chain or functional fragment or variant thereof. In some embodiments, the multimeric binding molecule can comprise a naturally occurring J-chain sequence, such as a mature human J-chain sequence (e.g., SEQ ID NO: 7). Alternatively, in some embodiments, the multimeric binding molecule can comprise a variant J-chain sequence, such as a variant sequence described herein with reduced glycosylation or reduced binding to one or more polymeric Ig receptors (e.g., pIgR, Fc alpha-mu receptor (FcαμR), or Fc mu receptor (FcμR)). See, e.g., US Patent Application Publication No. US20200239572, which is incorporated herein by reference in its entirety. In some embodiments, the multimeric binding molecule can comprise a functional fragment of a naturally occurring or variant J-chain. As persons of ordinary skill in the art will recognize, “a functional fragment” or a “functional variant” in this context includes those fragments and variants that can associate with binding units, e.g., IgM or IgA heavy chain constant regions, to form a pentameric IgM antibody, IgM-like antibody, or IgM-derived binding molecule or a dimeric IgA antibody, IgA-like antibody, or IgA-derived binding molecule, and/or can associate with certain immunoglobulin receptors, e.g., pIgR.

In certain embodiments, the J-chain can be modified, e.g., by introduction of a heterologous moiety, or two or more heterologous moieties, e.g., polypeptides, without interfering with the ability of binding molecule to assemble and bind to its binding target(s). See U.S. Pat. Nos. 9,951,134 and 10,400,038, and U.S. Patent Application Publication Nos. US-2019-0185570 and US-2018-0265596, each of which is incorporated herein by reference in its entirety.

Accordingly, a binding molecule described herein, including multispecific IgA, IgA-like, IgM, or IgM-like antibodies as described elsewhere herein, can comprise a modified J-chain or functional fragment or variant thereof comprising a heterologous moiety, e.g., a heterologous polypeptide, introduced, e.g., fused or chemically conjugated, into the J-chain or fragment or variant thereof. In certain embodiments, the heterologous moiety comprises the effector cell moiety. In certain embodiments, the heterologous polypeptide can be fused to the N-terminus of the J-chain or functional fragment or variant thereof, the C-terminus of the J-chain or functional fragment or variant thereof, or to both the N-terminus and C-terminus of the J-chain or functional fragment or variant thereof. In certain embodiments the heterologous polypeptide can be fused internally within the J-chain or functional fragment or variant thereof. In some embodiments, the heterologous polypeptide can be introduced into the J-chain at or near a glycosylation site. In some embodiments, the heterologous polypeptide can be introduced into the J-chain within about 10 amino acid residues from the C-terminus, or within about 10 amino acids from the N-terminus. In certain embodiments, the heterologous polypeptide can be introduced into the mature human J-chain of SEQ ID NO: 7 between cysteine residues 92 and 101 of SEQ ID NO: 7, or an equivalent location in a J-chain sequence, e.g., a J-chain variant or functional fragment of a J-chain. In a further embodiment, the heterologous polypeptide can be introduced into the mature human J-chain of SEQ ID NO: 7 at or near a glycosylation site. In a further embodiment, the heterologous polypeptide can be introduced into the mature human J-chain of SEQ ID NO: 7 within about 10 amino acid residues from the C-terminus, or within about 10 amino acids from the N-terminus.

In certain embodiments the heterologous moiety can be a peptide or polypeptide sequence fused in frame to the J-chain or chemically conjugated to the J-chain or fragment or variant thereof. In certain embodiments, the heterologous polypeptide is fused to the J-chain or functional fragment thereof via a peptide linker. Any suitable linker can be used, for example the peptide linker can include at least 5 amino acids, at least ten amino acids, and least 20 amino acids, at least 30 amino acids or more, and so on. In certain embodiments, the peptide linker includes least 5 amino acids, but no more than 25 amino acids. In certain embodiments the peptide linker can consist of 5 amino acids, 10 amino acids, 15 amino acids, amino acids, or 25 amino acids. In certain embodiments, the peptide linker consists of GGGGS (SEQ ID NO: 9), GGGGSGGGGS (SEQ ID NO: 10), GGGGSGGGGSGGGGS (SEQ ID NO: 11), GGGGS GGGGS GGGGS GGGGS (SEQ ID NO: 12), or GGGGS GGGGS GGGGS GGGGS GGGGS (SEQ ID NO: 13).

Heterologous moieties to be attached to a J-chain, such as a variant J-chain or fragment thereof disclosed herein, can include, without limitation, a binding moiety, e.g., an antibody or antigen-binding fragment thereof, e.g., a single chain Fv (scFv) molecule, a stabilizing peptide that can increase the half-life of the binding molecule, e.g., human serum albumin (HSA) or an HSA binding molecule, or a heterologous chemical moiety such as a polymer. In some embodiments, two heterologous moieties are attached to the J-chain, e.g., an effector cell engaging moiety and a half-life extending moiety, or two effector cell engaging moieties.

In some embodiments, a modified J-chain can comprise an antigen-binding domain that can include without limitation a polypeptide capable of specifically binding to a target antigen. In certain embodiments, an antigen-binding domain associated with a modified J-chain can be an antibody or an antigen-binding fragment thereof. In certain embodiments the antigen-binding domain can be a scFv antigen-binding domain or a single-chain antigen-binding domain derived, e.g., from a camelid or condricthoid antibody. In certain embodiments, the target is a target epitope, a target antigen, a target cell, or a target organ. In some embodiments, a J-chain-associated antigen binding domain specifically binds to an immune effector cell, e.g., a T cell such as a CD4+ T cell or a CD8+ cytotoxic T cell or an NK cell.

In certain embodiments, the binding domain, e.g., scFv fragment can specifically bind to CD3. In some embodiments, the binding domain, e.g., scFv fragment comprises a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 14 and SEQ ID NO: 15, SEQ ID NO: 16 and SEQ ID NO: 17, SEQ ID NO: 18 and SEQ ID NO: 19, SEQ ID NO: 20 and SEQ ID NO: 21, SEQ ID NO: 22 and SEQ ID NO: 23, SEQ ID NO: 24 and SEQ ID NO: 25, or SEQ ID NO: 26 and SEQ ID NO: 27, respectively with zero, one, or two single amino acid substitutions in one or more of the HCDRs or LCDRs.

In some embodiments, the binding domain, e.g., scFv fragment comprises an antibody VH and a VL, where the VH and VL comprise amino acid sequences at least 80%, at least 85%, at least 90%, at least 95% or 100% identical to SEQ ID NO: 14 and SEQ ID NO: 15, SEQ ID NO: 16 and SEQ ID NO: 17, SEQ ID NO: 18 and SEQ ID NO: 19, SEQ ID NO: 20 and SEQ ID NO: 21, SEQ ID NO: 22 and SEQ ID NO: 23, SEQ ID NO: 24 and SEQ ID NO: 25, or SEQ ID NO: 26 and SEQ ID NO: 27, respectively.

In some embodiments, the J-chain or fragment or variant thereof comprises an scFv fragment that can specifically bind to CD3 and comprises the amino acid sequence of SEQ ID NO: 32, SEQ ID NO: 33, SEQ ID NO: 36, SEQ ID NO:37, SEQ ID NO: 40, SEQ ID NO: 41, SEQ ID NO: 44, SEQ ID NO: 45, SEQ ID NO: 48, SEQ ID NO: 49, SEQ ID NO: 52, or SEQ ID NO: 53.

The antigen-binding domain can be introduced into the J-chain at any location that allows the binding of the antigen-binding domain to its binding target without interfering with J-chain function or the function of an associated multimeric binding molecule, e.g., a pentameric IgM or a dimeric or tetrameric IgA antibody. Insertion locations include but are not limited to at or near the C-terminus, at or near the N-terminus or at an internal location that, based on the three-dimensional structure of the J-chain, is accessible.

In some embodiments, the modified J-chain comprises an effector cell engaging moiety and a half-life extending moiety. In some embodiments, the effector cell engaging moiety is an scFv that binds CD3. In some embodiments, the half-life extending moiety is an albumin, such as a human serum albumin (HSA). In some embodiments, the J-chain or fragment or variant thereof comprises SEQ ID NO: 34, SEQ ID NO: 38, SEQ ID NO: 42, SEQ ID NO: 46, SEQ ID NO: 50, or SEQ ID NO: 54.

Variant J-Chains that Confer Increased Serum Half-Life

In certain embodiments, the J-chain is a functional variant J-chain that includes one or more single amino acid substitutions, deletions, or insertions relative to a reference J-chain identical to the variant J-chain except for the one or more single amino acid substitutions, deletions, or insertions. For example, certain amino acid substitutions, deletions, or insertions can result in the IgM-derived binding molecule exhibiting an increased serum half-life upon administration to a subject animal relative to a reference IgM-derived binding molecule that is identical except for the one or more single amino acid substitutions, deletions, or insertions in the variant J-chain, and is administered using the same method to the same animal species. In certain embodiments the variant J-chain can include one, two, three, or four single amino acid substitutions, deletions, or insertions relative to the reference J-chain.

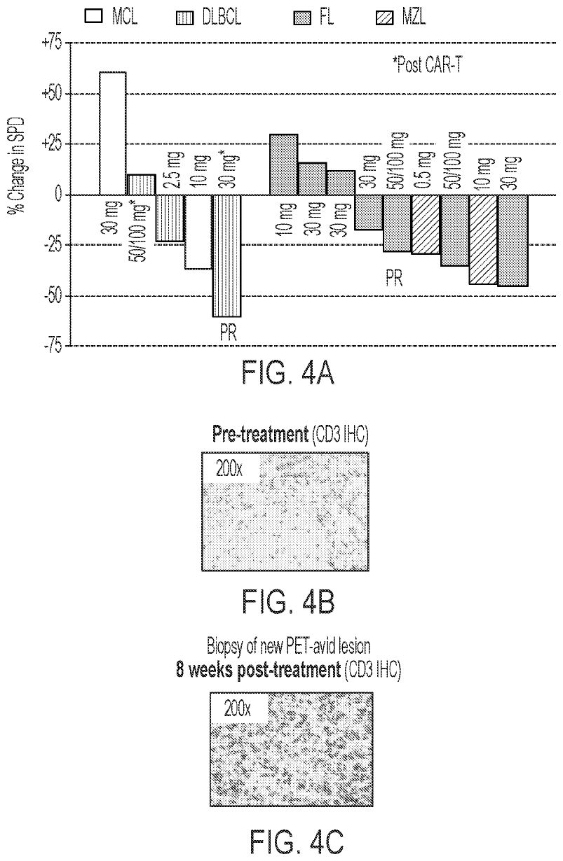

In certain embodiments, the J-chain, such as a modified J-chain, comprises an amino acid substitution at the amino acid position corresponding to amino acid Y102 of the mature wild-type human J-chain (SEQ ID NO: 7). By “an amino acid corresponding to amino acid Y102 of the mature wild-type human J-chain” is meant the amino acid in the sequence of the J-chain, which is homologous to Y102 in the human J-chain. For example, see US Patent Application Publication No. US20200239572, which is incorporated herein by reference in its entirety. The position corresponding to Y102 in SEQ ID NO: 7 is conserved in the J-chain amino acid sequences of at least 43 other species. See FIG. 4 of U.S. Pat. No. 9,951,134, which is incorporated by reference herein. Certain mutations at the position corresponding to Y102 of SEQ ID NO: 7 can inhibit the binding of IgM pentamers comprising the variant J-chain to certain immunoglobulin receptors, e.g., the human or murine Fcαμt receptor, the murine Fcμ, receptor, and/or the human or murine polymeric Ig receptor (pIgR).

A multimeric binding molecule comprising a mutation at the amino acid corresponding to Y102 of SEQ ID NO: 7 has an improved serum half-life when administered to an animal than a corresponding multimeric binding molecule that is identical except for the substitution, and which is administered to the same species in the same manner. In certain embodiments, the amino acid corresponding to Y102 of SEQ ID NO: 7 can be substituted with any amino acid. In certain embodiments, the amino acid corresponding to Y102 of SEQ ID NO: 7 can be substituted with alanine (A), serine (S) or arginine (R). In a particular embodiment, the amino acid corresponding to Y102 of SEQ ID NO: 7 can be substituted with alanine. In a particular embodiment the J-chain or functional fragment or variant thereof is a variant human J-chain referred to herein as “J*,” and comprises the amino acid sequence SEQ ID NO: 8.

Wild-type J-chains typically include one N-linked glycosylation site. In certain embodiments, a variant J-chain or functional fragment thereof of a multimeric binding molecule as provided herein includes a mutation within the asparagine(N)-linked glycosylation motif N-X₁-S/T, e.g., starting at the amino acid position corresponding to amino acid 49 (motif N6) of the mature human J-chain (SEQ ID NO: 7) or J* (SEQ ID NO: 8), where N is asparagine, X₁ is any amino acid except proline, and S/T is serine or threonine, and where the mutation prevents glycosylation at that motif. As demonstrated in see US Patent Application Publication No. US20200239572, mutations preventing glycosylation at this site can result in the multimeric binding molecule as provided herein, exhibiting an increased serum half-life upon administration to a subject animal relative to a reference multimeric binding molecule that is identical except for the mutation or mutations preventing glycosylation in the variant J-chain, and is administered in the same way to the same animal species.

For example, in certain embodiments the variant J-chain or functional fragment thereof of a pentameric IgM-derived or dimeric IgA-derived binding molecule as provided herein can include an amino acid substitution at the amino acid position corresponding to amino acid N49 or amino acid S51 of SEQ ID NO: 7 or SEQ ID NO: 8, provided that the amino acid corresponding to S51 is not substituted with threonine (T), or where the variant J-chain comprises amino acid substitutions at the amino acid positions corresponding to both amino acids N49 and S51 of SEQ ID NO: 7 or SEQ ID NO: 8. In certain embodiments, the position corresponding to N49 of SEQ ID NO: 7 or SEQ ID NO: 8 is substituted with any amino acid, e.g., alanine (A), glycine (G), threonine (T), serine (S) or aspartic acid (D). In a particular embodiment, the position corresponding to N49 of SEQ ID NO: 7 or SEQ ID NO: 8 can be substituted with alanine (A). In another embodiment, the position corresponding to N49 of SEQ ID NO: 7 or SEQ ID NO: 8 can be substituted with aspartic acid (D). In some embodiments, the position corresponding to S51 of SEQ ID NO: 7 or SEQ ID NO: 8 is substituted with alanine (A) or glycine (G). In some embodiments, the position corresponding to S51 of SEQ ID NO: 7 or SEQ ID NO: 8 is substituted with alanine (A).

### Variant IgM Constant Regions

IgM heavy chain constant regions of a multimeric binding molecule as described herein can be engineered to confer certain desirable properties to the multimeric binding molecules provided herein. For example, in certain embodiments, IgM heavy chain constant regions can be engineered to confer enhanced serum half-life to multimeric binding molecules as provided herein. Exemplary IgM heavy chain constant region mutations that can enhance serum half-life of an IgM-derived binding molecule are disclosed in see US Patent Application Publication No. US20200239572, which is incorporated by reference herein in its entirety. For example, a variant IgM heavy chain constant region of the IgM antibody, IgM-like antibody, or IgM-derived binding molecule as provided herein can include an amino acid substitution at a position corresponding to amino acid S401, E402, E403, 8344, and/or E345 of a wild-type human IgM constant region (e.g., SEQ ID NO: 1 or SEQ ID NO: 2). By “an amino acid corresponding to amino acid S401, E402, E403, 8344, and/or E345 of a wild-type human IgM constant region” is meant the amino acid in the sequence of the IgM constant region of any species which is homologous to S401, E402, E403, 8344, and/or E345 in the human IgM constant region. In certain embodiments, the amino acid corresponding to S401, E402, E403, 8344, and/or E345 of SEQ ID NO: 1 or SEQ ID NO: 2 can be substituted with any amino acid, e.g., alanine.

In certain embodiments, an IgM antibody, IgM-like antibody, or other IgM-derived binding molecule as provided herein, can be engineered to exhibit reduced complement-dependent cytotoxicity (CDC) activity to cells in the presence of complement, relative to a reference IgM antibody, IgM-like antibody, or other IgM-derived binding molecule with corresponding reference human IgM constant regions identical, except for the mutations conferring reduced CDC activity. These CDC mutations can be combined with any of the mutations to confer increased serum half-life as provided herein. By “corresponding reference human IgM constant region” is meant a human IgM constant region that is identical to the variant IgM constant region except for the modification or modifications in the constant region affecting CDC activity. In certain embodiments, the variant human IgM constant region includes one or more amino acid substitutions, e.g., in the Cμ3 domain, relative to a wild-type human IgM constant region as described, e.g., in PCT Publication No. WO/2018/187702, which is incorporated herein by reference in its entirety. Assays for measuring CDC are well known to those of ordinary skill in the art, and exemplary assays are described e.g., in PCT Publication No. WO/2018/187702.

In certain embodiments, a variant human IgM constant region conferring reduced CDC activity includes an amino acid substitution corresponding to the wild-type human IgM constant region at position L310, P311, P313, and/or K315 of SEQ ID NO: 1 (human IgM constant region allele IGHM*03) or SEQ ID NO: 2 (human IgM constant region allele IGHM*04). In certain embodiments, a variant human IgM constant region conferring reduced CDC activity includes an amino acid substitution corresponding to the wild-type human IgM constant region at position P311 of SEQ ID NO: 1 or SEQ ID NO: 2. In other embodiments the variant IgM constant region as provided herein contains an amino acid substitution corresponding to the wild-type human IgM constant region at position P313 of SEQ ID NO: 1 or SEQ ID NO: 2. In other embodiments the variant IgM constant region as provided herein contains a combination of substitutions corresponding to the wild-type human IgM constant region at positions P311 of SEQ ID NO: 1 or SEQ ID NO: 2 and P313 of SEQ ID NO: 1 or SEQ ID NO: 2. These proline residues can be independently substituted with any amino acid, e.g., with alanine, serine, or glycine. In certain embodiments, a variant human IgM constant region conferring reduced CDC activity includes an amino acid substitution corresponding to the wild-type human IgM constant region at position K315 of SEQ ID NO: 1 or SEQ ID NO: 2. The lysine residue can be independently substituted with any amino acid, e.g., with alanine, serine, glycine, or aspartic acid. In certain embodiments, a variant human IgM constant region conferring reduced CDC activity includes an amino acid substitution corresponding to the wild-type human IgM constant region at position K315 of SEQ ID NO: 1 or SEQ ID NO: 2 with aspartic acid.

Human and certain non-human primate IgM constant regions typically include five (5) naturally-occurring asparagine (N)-linked glycosylation motifs or sites. As used herein “an N-linked glycosylation motif” comprises or consists of the amino acid sequence N-X₁-S/T, where N is asparagine, X₁ is any amino acid except proline (P), and S/T is serine (S) or threonine (T). The glycan is attached to the nitrogen atom of the asparagine residue. See, e.g., Drickamer K, Taylor M E (2006), Introduction to Glycobiology (2nd ed.). Oxford University Press, USA. N-linked glycosylation motifs occur in the human IgM heavy chain constant regions of SEQ ID NO: 1 or SEQ ID NO: 2 starting at positions 46 (“N1”), 209 (“N2”), 272 (“N3”), 279 (“N4”), and 440 (“N5”). These five motifs are conserved in non-human primate IgM heavy chain constant regions, and four of the five are conserved in the mouse IgM heavy chain constant region. Accordingly, in some embodiments, IgM heavy chain constant regions of a multimeric binding molecule as provided herein comprise 5 N-linked glycosylation motifs: N1, N2, N3, N4, and N5. In some embodiments, at least three of the N-linked glycosylation motifs (e.g., N1, N2, and N3) on each IgM heavy chain constant region are occupied by a complex glycan.

In certain embodiments, at least one, at least two, at least three, or at least four of the N-X₁-S/T motifs can include an amino acid insertion, deletion, or substitution that prevents glycosylation at that motif. In certain embodiments, the IgM-derived multimeric binding molecule can include an amino acid insertion, deletion, or substitution at motif N1, motif N2, motif N3, motif N5, or any combination of two or more, three or more, or all four of motifs N1, N2, N3, or N5, where the amino acid insertion, deletion, or substitution prevents glycosylation at that motif. In some embodiment, the IgM constant region comprises one or more substitutions relative to a wild-type human IgM constant region at positions 46, 209, 272, or 440 of SEQ ID NO: 1 (human IgM constant region allele IGHM*03) or SEQ ID NO: 2 (human IgM constant region allele IGHM*04). See, e.g., PCT Application No. PCT/US2020/047495, filed Aug. 21, 2020, which is incorporated herein by reference in its entirety.

### Producing Multimeric Binding Molecules

The multimeric binding molecules used in certain methods disclosed herein can be produced from a host cell comprising one or more vectors encoding the polypeptide subunits of a multimeric binding molecule described herein using techniques known in the art. One vector can comprise nucleotide sequences encoding the heavy chain, light chain, and J-chain. Alternatively, two or three vectors can encode any combination of the heavy chain, light chain, and J-chain, such as one vector encoding the heavy chain and light chain, and a second vector encoding the J-chain or three vectors, one encoding the heavy chain, one encoding the light chain, and one encoding the J-chain.

This disclosure employs, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Green and Sambrook, ed. (2012) Molecular Cloning A Laboratory Manual (4th ed.; Cold Spring Harbor Laboratory Press); Sambrook et al., ed. (1992) Molecular Cloning: A Laboratory Manual, (Cold Springs Harbor Laboratory, NY); D. N. Glover and B. D. Hames, eds., (1995) DNA Cloning 2d Edition (IRL Press), Volumes 1-4; Gait, ed. (1990) Oligonucleotide Synthesis (IRL Press); Mullis et al. U.S. Pat. No. 4,683,195; Hames and Higgins, eds. (1985) Nucleic Acid Hybridization (IRL Press); Hames and Higgins, eds. (1984) Transcription And Translation (IRL Press); Freshney (2016) Culture Of Animal Cells, 7th Edition (Wiley-Blackwell); Woodward, J., Immobilized Cells And Enzymes (IRL Press) (1985); Perbal (1988) A Practical Guide To Molecular Cloning; 2d Edition (Wiley-Interscience); Miller and Calos eds. (1987) Gene Transfer Vectors For Mammalian Cells, (Cold Spring Harbor Laboratory); S. C. Makrides (2003) Gene Transfer and Expression in Mammalian Cells (Elsevier Science); Methods in Enzymology, Vols. 151-155 (Academic Press, Inc., N.Y.); Mayer and Walker, eds. (1987) Immunochemical Methods in Cell and Molecular Biology (Academic Press, London); Weir and Blackwell, eds.; and in Ausubel et al. (1995) Current Protocols in Molecular Biology (John Wiley and Sons).

General principles of antibody engineering are set forth, e.g., in Strohl, W. R., and L. M. Strohl (2012), Therapeutic Antibody Engineering (Woodhead Publishing). General principles of protein engineering are set forth, e.g., in Park and Cochran, eds. (2009), Protein Engineering and Design (CDC Press). General principles of immunology are set forth, e.g., in: Abbas and Lichtman (2017) Cellular and Molecular Immunology 9th Edition (Elsevier). Additionally, standard methods in immunology known in the art can be followed, e.g., in Current Protocols in Immunology (Wiley Online Library); Wild, D. (2013), The Immunoassay Handbook 4th Edition (Elsevier Science); Greenfield, ed. (2013), Antibodies, a Laboratory Manual, 2d Edition (Cold Spring Harbor Press); and Ossipow and Fischer, eds., (2014), Monoclonal Antibodies: Methods and Protocols (Humana Press).

All of the references cited above, as well as all references cited herein, are incorporated herein by reference in their entireties.

### EXEMPLARY EMBODIMENTS

Among the provided embodiments are:

Embodiment 1. A method of treating cancer in a subject in need thereof, comprising:
(a) administering a dosage of 10 mg to 50 mg of a T cell engaging molecule to the subject;
(b) administering a dosage of 75 mg to 600 mg of the T cell engaging molecule to the subject at least 5 days after the administration of step (a),
wherein the T cell engaging molecule is a multimeric binding molecule comprising five bivalent binding units and a modified J-chain, wherein each binding unit comprises two IgM heavy chains, each comprising a heavy chain variable region (VH) and an IgM constant region and two light chains, each comprising a light chain variable region (VL) and a light chain constant region,
wherein an associated VH and VL specifically bind to CD20 or a subunit thereof,
wherein the VH comprises three immunoglobulin complementarity determining regions: HCDR1, HCDR2, and HCDR3, and the VL comprises three immunoglobulin complementarity determining regions: LCDR1, LCDR2, and LCDR3, wherein the HCDR1, HCDR2, HCDR3, LCDR1, LCDR2, and LCDR3 comprise, respectively, the amino acid sequence of SEQ ID NO: 55, SEQ ID NO: 56; SEQ ID NO: 57; SEQ ID NO: 58; SEQ ID NO: 59; and SEQ ID NO: 60,
wherein the modified J-chain comprises a J-chain or functional fragment or variant thereof and an scFv molecule that specifically binds to CD3,
and wherein the scFv comprises the amino acid sequence of SEQ ID NO: 31.

Embodiment 2. The method of embodiment 1, further comprising (c) administering the dosage of the T cell engaging molecule administered to the subject in step (b) at least 5 days after the administration of step (b).

Embodiment 3. The method of embodiment 1 or embodiment 2, wherein the dosage of the T cell engaging molecule in step (b) is 100 mg to 300 mg.

Embodiment 4. The method of embodiment 3, wherein the dosage of the T cell engaging molecule in step (b) is 100 mg.

Embodiment 5. The method of embodiment 3, wherein the dosage of the T cell engaging molecule in step (b) is 300 mg.

Embodiment 6. A method of treating cancer in a subject in need thereof, comprising administering a dosage of 75 mg to 600 mg of the T cell engaging molecule to the subject,
wherein the T cell engaging molecule is a multimeric binding molecule comprising five bivalent binding units and a modified J-chain, wherein each binding unit comprises two IgM heavy chains, each comprising a heavy chain variable region (VH) and an IgM constant region and two light chains, each comprising a light chain variable region (VL) and a light chain constant region,
wherein an associated VH and VL specifically bind to CD20 or a subunit thereof,
wherein the VH comprises three immunoglobulin complementarity determining regions: HCDR1, HCDR2, and HCDR3, and the VL comprises three immunoglobulin complementarity determining regions: LCDR1, LCDR2, and LCDR3, wherein the HCDR1, HCDR2, HCDR3, LCDR1, LCDR2, and LCDR3 comprise, respectively, the amino acid sequence of SEQ ID NO: 55, SEQ ID NO: 56; SEQ ID NO: 57; SEQ ID NO: 58; SEQ ID NO: 59; and SEQ ID NO: 60,
wherein the modified J-chain comprises a J-chain or functional fragment or variant thereof and an scFv molecule that specifically binds to CD3,
and wherein the scFv comprises the amino acid sequence of SEQ ID NO: 31.

Embodiment 7. The method of embodiment 6, wherein the dosage is 100 mg to 300 mg.

Embodiment 8. The method of embodiment 7, wherein the dosage is 100 mg.

Embodiment 9. The method of embodiment 7 or embodiment 8, wherein the subject had received a prior dosage of the T cell engaging molecule, wherein the prior dosage was less than 100 mg.

Embodiment 10. The method of embodiment 9, wherein the prior dosage was 10-50 mg.

Embodiment 11. The method of embodiment 9, wherein the prior dosage comprised two or three prior dosages of the T cell engaging molecule, wherein the two or three prior dosages were less than 100 mg.

Embodiment 12. A method of treating cancer in a subject in need thereof, comprising:
(a) administering to the subject a T cell engaging molecule at a determined dosage;
(b) monitoring the subject for an administration-related symptom; and
(c)(i) administering the T cell engaging molecule to the subject at the same or a reduced dosage relative to the determined dosage if the subject had the administration-related symptom, and
(ii) administering the T cell engaging molecule to the subject at an increased dosage relative to the determined dosage if the subject did not have the administration-related symptom,
wherein the T cell engaging molecule is a multimeric binding molecule comprising five bivalent binding units and a modified J-chain, wherein each binding unit comprises two IgM heavy chains, each comprising a heavy chain variable region (VH) and an IgM constant region and two light chains, each comprising a light chain variable region (VL) and a light chain constant region,
wherein an associated VH and VL specifically bind to CD20 or a subunit thereof,
wherein the VH comprises three immunoglobulin complementarity determining regions: HCDR1, HCDR2, and HCDR3, and the VL comprises three immunoglobulin complementarity determining regions: LCDR1, LCDR2, and LCDR3, wherein the HCDR1, HCDR2, HCDR3, LCDR1, LCDR2, and LCDR3 comprise, respectively, the amino acid sequence of SEQ ID NO: 55; SEQ ID NO: 56; SEQ ID NO: 57; SEQ ID NO: 58; SEQ ID NO: 59; and SEQ ID NO: 60,
wherein the modified J-chain comprises a J-chain or functional fragment or variant thereof and an scFv molecule that specifically binds to CD3,
wherein the scFv comprises the amino acid sequence of SEQ ID NO: 31.

Embodiment 13. The method of embodiment 12, wherein the administration-related symptom comprises hypotension, chills, fever, elevated C reactive protein (CRP) level, fatigue, nausea, vomiting, insomnia, back pain, cytokine release, or a combination thereof.

Embodiment 14. The method of embodiment 13, wherein the administration-related symptom comprises hypotension, chills, fever, elevated C reactive protein (CRP) level, or a combination thereof.

Embodiment 15. The method of any one of embodiments 12 to 14, further comprising: (d) repeating steps (b) and (c) one or more times.

Embodiment 16. The method of any one of embodiments 12 to 15, wherein the increased dosage of the T cell engaging molecule in step (c)(ii) is 25%-1000% greater than the determined dosage of the T cell engaging molecule.

Embodiment 17. The method of any one of embodiments 12 to 15, wherein the increased dosage of the T cell engaging molecule in step (c)(ii) is 30 mg to 300 mg.

Embodiment 18. The method of embodiment 17, wherein the increased dosage of the T cell engaging molecule in step (c)(ii) is 100 mg to 300 mg.

Embodiment 19. The method of any one of embodiments 18, wherein the increased dosage of the T cell engaging molecule in step (c)(ii) is 100 mg or 300 mg.

Embodiment 20. The method of any one of embodiments 12 to 19, wherein step (a), step (c)(i), and/or step (c)(ii) further comprises administering dexamethasone.

Embodiment 21. The method of any one of embodiments 1 to 20, wherein the subject had previously received a different cancer therapy.

Embodiment 22. The method of embodiment 21, wherein the different cancer therapy comprises chemotherapy, radiation therapy, or immunotherapy, wherein the immunotherapy is different from the T cell engaging molecule.

Embodiment 23. The method of embodiment 22, wherein the immunotherapy is rituximab.

Embodiment 24. A method of treating cancer in a subject in need thereof, the method comprising administering a T cell engaging molecule to the subject,
wherein the T cell engaging molecule is a multimeric binding molecule comprising five bivalent binding units and a modified J-chain, wherein each binding unit comprises two IgM heavy chains, each comprising a heavy chain variable region (VH) and an IgM constant region and two light chains, each comprising a light chain variable region (VL) and a light chain constant region,
wherein an associated VH and VL specifically bind to CD20 or a subunit thereof,
wherein the VH comprises three immunoglobulin complementarity determining regions: HCDR1, HCDR2, and HCDR3, and the VL comprises three immunoglobulin complementarity determining regions: LCDR1, LCDR2, and LCDR3, wherein the HCDR1, HCDR2, HCDR3, LCDR1, LCDR2, and LCDR3 comprise, respectively, the amino acid sequence of SEQ ID NO: 55, SEQ ID NO: 56; SEQ ID NO: 57; SEQ ID NO: 58; SEQ ID NO: 59; and SEQ ID NO: 60,
wherein the modified J-chain comprises a J-chain or functional fragment or variant thereof and an scFv molecule that specifically binds to CD3,
wherein the scFv comprises the amino acid sequence of SEQ ID NO: 31, and
wherein the subject had previously been administered rituximab.

Embodiment 25. A method of treating cancer in a subject in need thereof, the method comprising
administering rituximab to the subject, and
administering a T cell engaging molecule to the subject,
wherein the T cell engaging molecule is a multimeric binding molecule comprising five bivalent binding units and a modified J-chain, wherein each binding unit comprises two IgM heavy chains, each comprising a heavy chain variable region (VH) and an IgM constant region and two light chains, each comprising a light chain variable region (VL) and a light chain constant region,
wherein an associated VH and VL specifically bind to CD20 or a subunit thereof,
wherein the VH comprises three immunoglobulin complementarity determining regions: HCDR1, HCDR2, and HCDR3, and the VL comprises three immunoglobulin complementarity determining regions: LCDR1, LCDR2, and LCDR3, wherein the HCDR1, HCDR2, HCDR3, LCDR1, LCDR2, and LCDR3 comprise, respectively, the amino acid sequence of SEQ ID NO: 55, SEQ ID NO: 56; SEQ ID NO: 57; SEQ ID NO: 58; SEQ ID NO: 59; and SEQ ID NO: 60,
wherein the modified J-chain comprises a J-chain or functional fragment or variant thereof and an scFv molecule that specifically binds to CD3, and
wherein the scFv comprises the amino acid sequence of SEQ ID NO: 31.

Embodiment 26. The method of any one of embodiments 23 to 25, wherein the administering of the T cell engaging molecule occurs at least 1 week, at least 2 weeks, at least 1 month, at least 2 months, at least 3 months, at least 6 months, at least 9 months, at least 1 year, or at least 2 years after the rituximab was administered.

Embodiment 27. The method of any one of embodiments 23 to 25, wherein the subject has rituximab serum levels of 0 ng/mL to 200 ng/mL.

Embodiment 28. The method of embodiment 27, wherein the subject has rituximab serum levels of 0 ng/mL to 100 ng/mL.

Embodiment 29. The method of any one of embodiments 23 to 28, wherein the rituximab administration comprises administering a dose of 375 mg/m 2 of rituximab.

Embodiment 30. The method of any one of embodiments 23 to 29, wherein the rituximab administration comprises administering rituximab once weekly for 4 to 8 weeks.

Embodiment 31. The method of embodiment 22, wherein the immunotherapy is chimeric antigen receptor (CAR)-expressing T cells.

Embodiment 32. The method of any one of embodiments 1 to 31, wherein the method further comprises administering chimeric antigen receptor (CAR)-expressing T cells to the subject.

Embodiment 33. A method of treating cancer in a subject in need thereof, the method comprising:
administering chimeric antigen receptor (CAR)-expressing T cells to the subject; and
administering a T cell engaging molecule to the subject,
wherein the T cell engaging molecule is a multimeric binding molecule comprising five bivalent binding units and a modified J-chain, wherein each binding unit comprises two IgM heavy chains, each comprising a heavy chain variable region (VH) and an IgM constant region and two light chains, each comprising a light chain variable region (VL) and a light chain constant region,
wherein an associated VH and VL specifically bind to CD20 or a subunit thereof,
wherein the VH comprises three immunoglobulin complementarity determining regions: HCDR1, HCDR2, and HCDR3, and the VL comprises three immunoglobulin complementarity determining regions: LCDR1, LCDR2, and LCDR3, wherein the HCDR1, HCDR2, HCDR3, LCDR1, LCDR2, and LCDR3 comprise, respectively, the amino acid sequence of SEQ ID NO: 55; SEQ ID NO: 56; SEQ ID NO: 57; SEQ ID NO: 58; SEQ ID NO: 59; and SEQ ID NO: 60,
wherein the modified J-chain comprises a J-chain or functional fragment or variant thereof and an scFv molecule that specifically binds to CD3,
and wherein the scFv comprises the amino acid sequence of SEQ ID NO: 31.

Embodiment 34. A method of treating cancer in a subject in need thereof, the method comprising administering a T cell engaging molecule to the subject, wherein the T cell engaging molecule is a multimeric binding molecule comprising five bivalent binding units and a modified J-chain, wherein each binding unit comprises two IgM heavy chains, each comprising a heavy chain variable region (VH) and an IgM constant region and two light chains, each comprising a light chain variable region (VL) and a light chain constant region,
wherein an associated VH and VL specifically bind to CD20 or a subunit thereof,
wherein the VH comprises three immunoglobulin complementarity determining regions: HCDR1, HCDR2, and HCDR3, and the VL comprises three immunoglobulin complementarity determining regions: LCDR1, LCDR2, and LCDR3, wherein the HCDR1, HCDR2, HCDR3, LCDR1, LCDR2, and LCDR3 comprise, respectively, the amino acid sequence of SEQ ID NO: 55; SEQ ID NO: 56; SEQ ID NO: 57; SEQ ID NO: 58; SEQ ID NO: 59; and SEQ ID NO: 60,
wherein the modified J-chain comprises a J-chain or functional fragment or variant thereof and an scFv molecule that specifically binds to CD3,
wherein the scFv comprises the amino acid sequence of SEQ ID NO: 31, and
wherein the subject had previously been administered chimeric antigen receptor (CAR)-expressing T cells.

Embodiment 35. The method of any one of embodiments 31 to 34, wherein the CAR-T cells are lisocabtagene maraleucel, axicabtagene ciloleucel, tisagenlecleucel, or brexucabtagene autoleucel.

Embodiment 36. The method of any one of embodiments 1 to 35, wherein the IgM heavy chain constant regions or multimerizing fragments or variants thereof are human IgM constant regions.

Embodiment 37. The method of embodiment 36, wherein the IgM heavy chain constant regions each comprise the amino acid sequence SEQ ID NO: 1, SEQ ID NO: 2, or a multimerizing fragment or variant thereof.

Embodiment 38. The method of any one of embodiments 1 to 37, wherein the J-chain or functional fragment or variant thereof comprises SEQ ID NO: 7.

Embodiment 39. The method of any one of embodiments 1 to 38, wherein the modified J-chain further comprises an albumin.

Embodiment 40. The method of embodiment 39, wherein the albumin comprises human serum albumin.

Embodiment 41. The method of embodiment 40, wherein the J-chain or fragment or variant thereof comprises SEQ ID NO: 34.

Embodiment 42. The method of any one of embodiments 1 to 41, wherein the heavy chain comprises SEQ ID NO: 61 and the light chain comprises SEQ ID NO: 62.

Embodiment 43. The method of any one of embodiments 1 to 42, wherein the subject is human.

Embodiment 44. The method of any one of embodiments 1 to 43, wherein the cancer is a CD20 positive cancer.

Embodiment 45. The method of any one of embodiments 1 to 44, wherein the cancer is a leukemia, lymphoma, or myeloma.

Embodiment 46. The method of any one of embodiments 1 to 45, wherein the cancer is non-Hodgkin lymphoma (NHL).

Embodiment 47. The method of embodiment 46, wherein the NHL is diffuse large B-cell lymphoma (DLBCL), follicular lymphoma (FL), mantle cell lymphoma (MCL), or marginal zone lymphoma (MZL).

Embodiment 48. The method of any one of embodiments 1 to 47, wherein the cancer is relapsed or refractory cancer.

The following examples are offered by way of illustration and not by way of limitation.

### EXAMPLES

### Example 1: Phase I Clinical Trial

Methods

Patients with CD20-positive relapsed/refractory (R/R) non-Hodgkin lymphoma (NHL) were enrolled in a first in human, dose escalation Phase I study (ClinicalTrials.gov Identifier: NCT04082936). Patients were required to have had ≥2 prior lines of therapy, including an anti-CD20 mAb. Patients that were not refractory to prior CAR-T therapy were allowed with approval from medical monitor. Table 3 shows a summary of enrolled patient characteristics.

TABLE 3
Patient Characteristics

| Characteristic | Indolent (n = 10) | Aggressive (n = 6) |
|---|---|---|
| Median age (range) | 66.5 (47-75) | 61 (46-82) |
| Histology | FL = 8; MZL = 2 | DLBCL = 4; MCL = 2 |
| Prior therapies, median (range) | 4 (2-6) | 4 (2-6) |
| Prior auto SCT, n (%) | 2 (20%) | 2 (33%) |
| Prior CAR-T, n (%) | 1 (10%) | 3 (50%) |

DLBCL: diffuse large B-cell lymphoma, FL: follicular lymphoma, MCL: mantle cell lymphoma, MZL: marginal zone lymphoma, CAR-T: chimeric antigen receptor T-cell, SCT: stem cell transplant

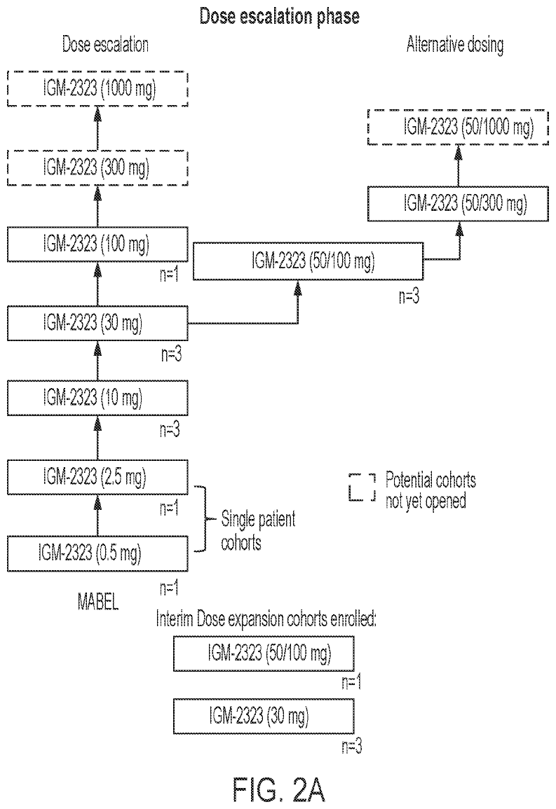

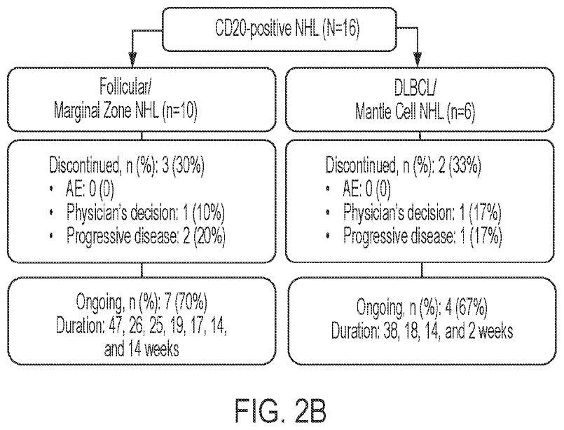

The current or already completed cohorts and planned cohorts are shown in FIG. 2A. Patients received IGM-2323, prepared as described in U.S. Pat. No. 10,618,978, intravenously on a weekly basis for a 21-day cycle. Patients in the dose escalation group received, or will receive, the amount of IGM-2323 according to their assigned cohort for multiple doses.

Patients in the alternative dosing group received one or two doses of the initial amount of their assigned cohort, then the dose was increased if the patient had not had fever, chills, or an elevated C-reactive protein (CRP) level. If the patient had any of these symptoms, the dose at the prior infusion was repeated. This method was repeated until the maximum dose for the cohort was reached. FIG. 2A shows the starting dose and maximum dose per cohort. This alternative dosing can allow for the optimization of individual immune activation while preserving or amplifying T cell activity and function over time.

Intra-subject dose escalation was permitted post-tumor assessment in the dose escalation group, and the new dose was the highest cleared dose. Subjects received up to 10 mg dexamethasone premedication for first infusion only and subsequent premedication was per investigator discretion. FIG. 2B shows the number of patients that have continued or discontinued treatment.

Results

These results include study patients enrolled and treated prior to Oct. 30, 2020 (data cut-off). Table 4 shows a summary of adverse events (AEs) that occurred after treatment with IGM-2323. AEs were graded using the NCI Common Toxicity Criteria for Adverse Events (CTCAE) version 5.0. (available on World Wide Web at ctep.cancer.gov/protocolDevelopment/electronic applications/ctc.htm).

TABLE 4
Adverse Events Occurring in ≥20% of patients

| | Any | | | |
|---|---|---|---|---|
| Preferred Term | grade | Grade 1 | Grade 2 | Grade ≥3 |
| (n = 16 patients) | (%) | (%) | (%) | (%) |
| Fatigue | 9 (56) | 8 (50) | 1 (6) | 0 |
| Hypophosphatemia | 7 (44) | 3 (19) | 4 (25) | 0 |
| Chills | 6 (38) | 4 (25) | 2 (13) | 0 |
| Pyrexia | 6 (38) | 4 (25) | 1 (6) | 0 |
| CRS | 4 (25) | 3 (19) | 1 (6) | 0 |
| Infusion related | 4 (25) | 2 (13) | 2 (13) | 0 |
| reaction | | | | |
| Anemia | 4 (25) | 2 (13) | 2 (13) | 0 |

IGM-2323 was well tolerated with no dose limiting toxicities, no immune cell associated neurotoxicity (ICANS) symptoms, and no Grade 3 or higher cytokine release syndrome (CRS). Four out of 5 patients with creatinine increase were assessed as unrelated to study treatment. CRS grading was by American Society for Transplantation and Cellular Therapy (ASTCT) criteria, and all CRS patients were also captured under pyrexia and/or chills. Three of 4 infusion related reaction (IRR) patients were also captured under CRS. Investigators observed one Grade 2 CRS, but only two CRS events required treatment with post-infusion steroids (usually just acetaminophen). No CRS was observed in the three patients treated in 50/100 cohort.

A single patient with pre-existing severe hypertension on four anti-hypertensive medications treated at 100 mg Cycle 1 Day 1 dose experienced Grade 2 CRS, chills, and increased creatinine and Grade 1 pyrexia, fatigue, and hypophosphatemia after the first infusion. No CRS symptoms were observed at subsequent infusions of study drug up to 100 mg, with or without dexamethasone pre-medication. This patient had cytokine elevation after Cycle 1 Day 1 dose and had a best response of stable disease (SD) (+6%) but was not included in further analyses.

One patient enrolled 2 weeks prior to data cut-off was also included in Table 3 but was not included in further analyses.

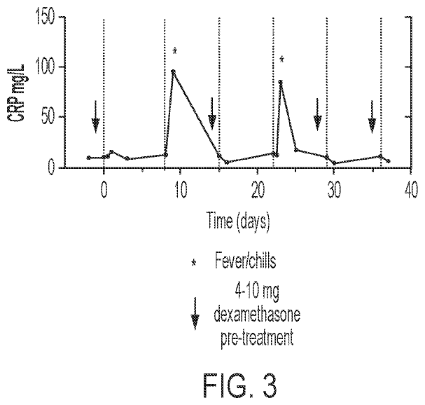

Regarding the observed post-infusion chills and/or fever observed in a subset of patients, the events were transient (≤3 hours), low grade, and associated with CRP elevations at 24 hours. For the subset of patients with these symptoms, recurrence of the symptoms was prevented with low dose dexamethasone premedication on subsequent infusions. An example of the association of CRP levels and fever/chills, as well as the ability of dexamethasone pre-treatment to prevent such symptoms are demonstrated in FIG. 3. The patient was in the 30 mg dosing cohort. For this patient, increased CRP levels and fever/chills directly correlated. Additionally, 10 mg of dexamethasone was administered for the first, third, and fifth doses, but not for the second and fourth doses. Administration of dexamethasone prevented the elevated CRP, fever, and chills for this patient.

A waterfall plot of the best overall response for each patient is shown in FIG. 4A. Nine of 14 patients showed evidence of tumor size reduction, and 12/14 having SD. One of 3 DLBCL, and one of 7 FL had a partial response. Eight of 9 patients with indolent NHL achieved best overall response of stable disease. Additionally, B cell depletion/reduction was observed in 6 of 6 patients with circulating B cells at baseline. The PR from the 50/100 mg cohort shown in FIG. 4A had lymph node size decreased to below normal with marked decrease in positron emission tomography (PET) activity. The PR cut-off was determined according to the Lugano 2014 criteria (Cheson et al., 2014, J Clin Oncol, 32(27): 3059-3067).

Out of 14 patients, two patients had prior CAR-T therapy. One of these patients was 63 years old, had R/R DLBCL, and was assigned to the 30 mg cohort. FIG. 4B shows an anti-CD3 immunohistochemistry (IHC) image of a pre-treatment tumor and FIG. 4C shows an anti-CD3 IHC image of a new PET-avid lesion at 8 weeks. The biopsy at 8 weeks shows intense T cell infiltration with only scant lymphoma cells. The biopsy also found >95% CD3+ T cell infiltrates by flow cytometry post-treatment. The lesion completely resolved by positron emission tomography-computerized tomography (PET-CT) at 12 weeks (data not shown).

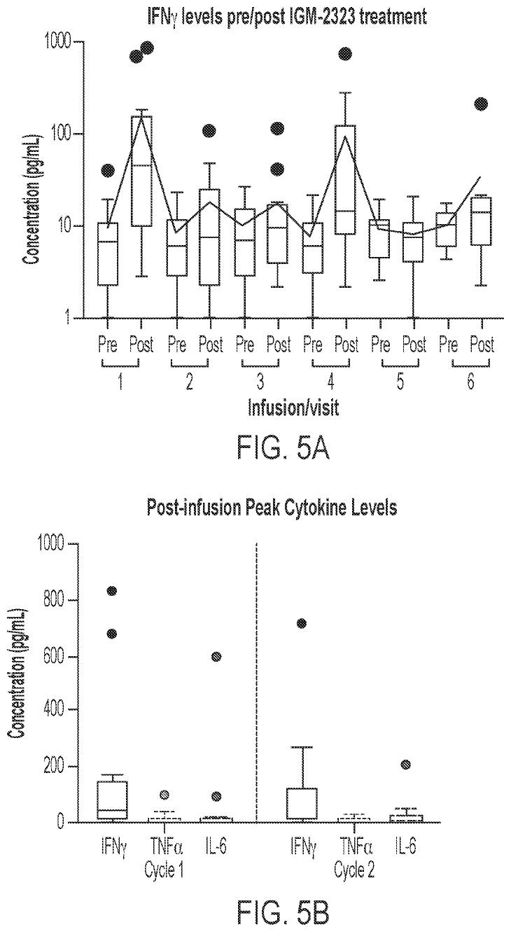

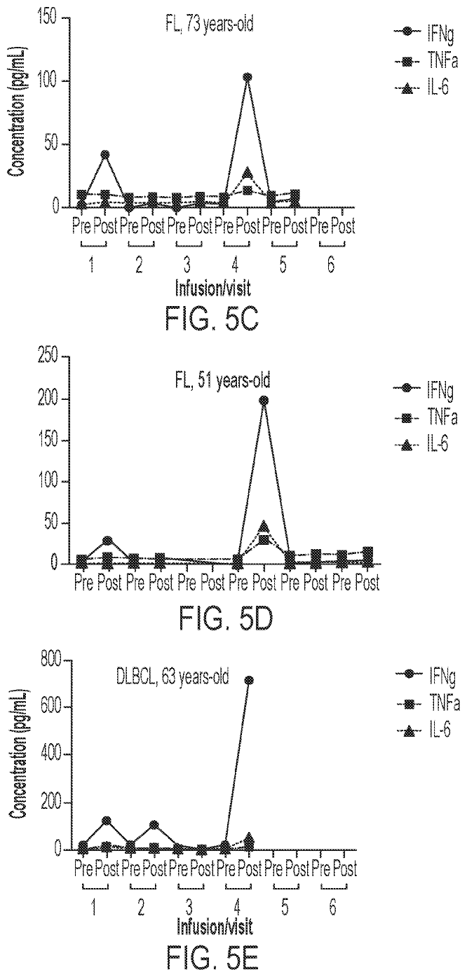

Cytokine levels in frozen plasma were assessed at a central laboratory. FIGS. 5A-5B (14 patients) and FIGS. 5C-5E (exemplary individual patients) show the highest concentrations obtained during dense sampling up to 72 hours for Infusion 1 and 4, and sparse sampling up to 24 hours for Infusions 2, 3, 5, and 6.

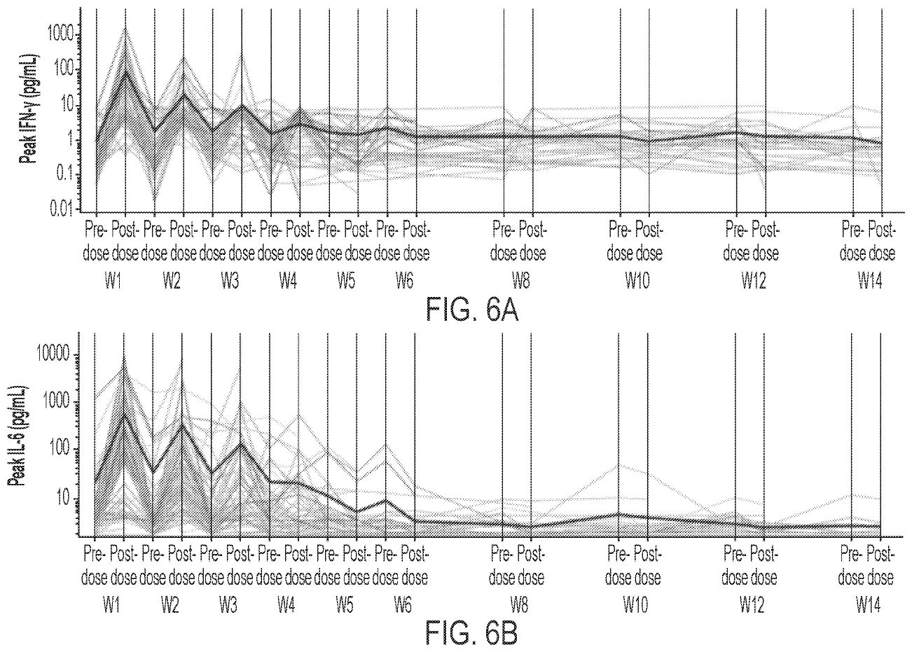

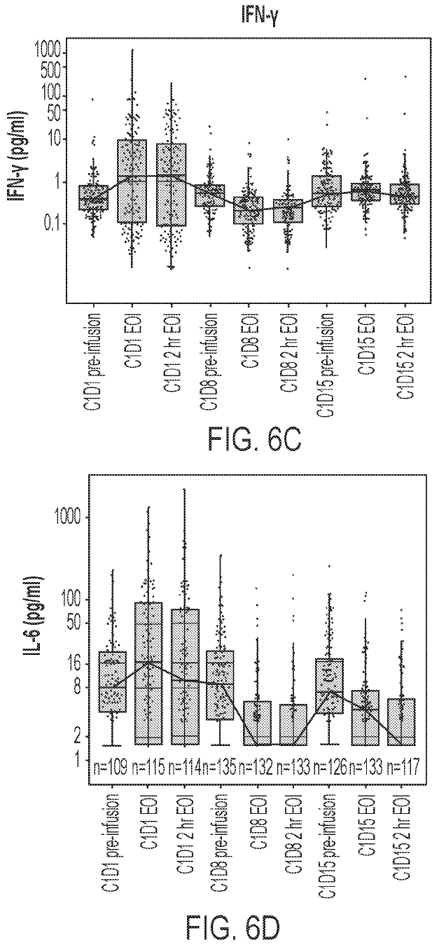

IGM-2323 treatment led to transient, repeatable, and IFNγ-dominant immune activity. IFNγ was detectable above baseline levels in 12/12 patients treated at ≥10 mg IGM-2323. In most patients treated IFNγ levels were much greater than IL-6 and TNFα. For patients dosed with ≥30 mg IGM-2323, 9/9 show repeatable IFNγ spikes and 4/9 show higher IFNγ spikes at later infusions suggesting that IGM-2323 can maintain or build up T cell activity over time. This is a different pattern compared to traditional IgG T cell engagers, such as odronextamab as reported by Brouwer-Visser et al. (25^{th} European Hematology Association (EHA) Virtual Congress, Jun. 11-21, 2020, poster EP1270) and mosunetuzumab as reported by Hernandez et al. (61^{st} ASH Annual Meeting & Exposition, Dec. 7-10, 2019, Orlando, Florida, USA, poster P-1585). According to Brouwer-Visser et al., patients treated with odronextamab exhibited increased levels of IFNγ, IL-6, IL-10, G-CSF, and TNFα between end of induction and 4 hours after first split infusion. According to the plots of IFNγ and IL-6 serum levels in Brouwer-Visser et al., reproduced as FIGS. 6A and 6B, the initial average peak IL-6 concentration was ˜9× greater than the initial average peak IFNγ concentration. Additionally, “subsequent post-dosing cytokine peaks continuously decreased with each treatment week.” Similarly, as reproduced in FIGS. 6C and 6D, Hernandez et al. showed that the initial average peak IL-6 concentration was ˜8× greater than the initial average peak IFNγ concentration in patients treated with mosunetuzumab.

### Example 2: Phase I Clinical Trial Update

Methods

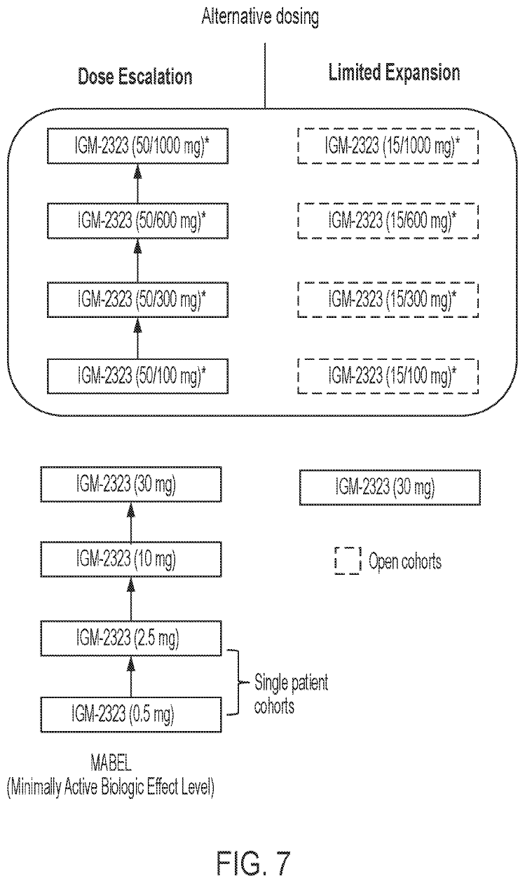

The clinical trial described in Example 1 was continued with additional patients. The current or already completed cohorts and planned cohorts are shown in FIG. 7. Patients in the alternative dosing group received one or two doses of the initial amount of their assigned cohort, then the dose was increased if the patient had not had hypotension, fever, chills, or an elevated C-reactive protein (CRP) level. If the patient had any of these symptoms, the dose at the prior infusion was repeated. This method was repeated until the maximum dose for the cohort was reached. FIG. 7 shows the starting dose and maximum dose per cohort, and Table 5 shows the planned doses in each cohort if the patient did not have one of the symptoms.

TABLE 5
Planned Doses in Each Cohort

| Dose Level (mg) | N |
|---|---|
| 0.5 | 1 |
| 2.5 | 1 |
| 10 | 3 |
| 30 | 6 |
| 100 | 1 |
| 15/30/50/100 | 1 |
| 15/50/100 | 3 |
| 15/50/100/300 | 2 |
| 15/50/100/600 | 0 |
| 15/50/100/1000 | 2 |
| 50/50/100 | 7 |
| 50/100/200 | 1 |
| 50/100/200/300 | 3 |
| 50/100/300 | 1 |
| 50/100/600 | 5 |
| 50/100/1000 | 3 |

Intra-subject dose escalation was permitted post-tumor assessment in the dose escalation group, and the new dose was the highest cleared dose. Subjects received up to 20 mg dexamethasone premedication for first infusion only and subsequent premedication was per investigator discretion.

Results

These results include study patients enrolled and treated prior to Sep. 10, 2021 (data cut-off). The baseline characteristics of all patients dosed as of the data cut-off are shown in Table 6.

TABLE 6
Baseline Characteristics

N = 40 (%)

Age at consent (years)

| | |
|---|---|
| Median (Range) | 64 (36-84) |

Tumor Type

| | |
|---|---|
| DLBCL | 18 (45%) |
| FL | 14 (35%) |
| MZL | 4 (10.0%) |
| MCL | 4 (10.0%) |

Gender

| | |
|---|---|
| Male | 28 (70.0%) |
| Female | 12 (30.0%) |

Prior ASCT

| | |
|---|---|
| Yes | 3 (8%) |
| No | 37 (92.0%) |

Prior CAR-T therapy

| | |
|---|---|
| Yes | 8 (20.0%) |
| No | 32 (80.0%) |

Prior Lines of Treatment

| | |
|---|---|
| 2 | 14 (35%) |
| 3 | 10 (25%) |
| 4 or more | 16 (40%) |

Disease stage at study entry, n (%)

| | |
|---|---|
| I-II | 7 (18%) |
| III-IV | 33 (82%) |

Table 7 and Table 8 show a summary of adverse events of special interest, which includes cytokine release syndrome (CRS), infusion related reaction (IRR) immune effector cell-associated neurotoxicity syndrome (ICANS), and neutropenia adverse events (AEs) that occurred after treatment with IGM-2323 from all patients and for the subset of patients dosed with the alternative dosing, respectively. AEs were graded using the NCI Common Toxicity Criteria for Adverse Events (CTCAE) version 5.0. (available on World Wide Web at ctep.cancer.gov/protocolDevelopment/electronic_applications/ctc.htm).

TABLE 7
Adverse Events of Special Interest in All Patients

| All Patients | Grade 1 | Grade 2 | Grade 3 | Grade 4 | Grade 5 |
|---|---|---|---|---|---|
| (N = 40) | n (%) | n (%) | n (%) | n (%) | n (%) |
| CRS | 7 (18%) | 2 (5%) | 1 (3%) | 0 | 0 |
| IRR | 3 (8%) | 7 (18%) | 2 (5%) | 0 | 0 |
| ICANS | 0 | 0 | 0 | 0 | 0 |
| Neutropenia | 1 (3%) | 0 | 1 (3%) | 1 (3%) | 0 |

TABLE 8
Adverse Events of Special Interest in Alternatively Dosed Patients

| Alternative | | | | | |
|---|---|---|---|---|---|
| Dosing | | | | | |
| Cohorts | Grade 1 | Grade 2 | Grade 3 | Grade 4 | Grade 5 |
| (N = 28) | n (%) | n (%) | n (%) | n (%) | n (%) |
| CRS | 3 (11%) | 1 (4%) | 1 (4%) | 0 | 0 |
| IRR | 3 (11%) | 3 (11%) | 1 (4%) | 0 | 0 |
| ICANS | 0 | 0 | 0 | 0 | 0 |
| Neutropenia | 1 (4%) | 0 | 0 | 0 | 0 |

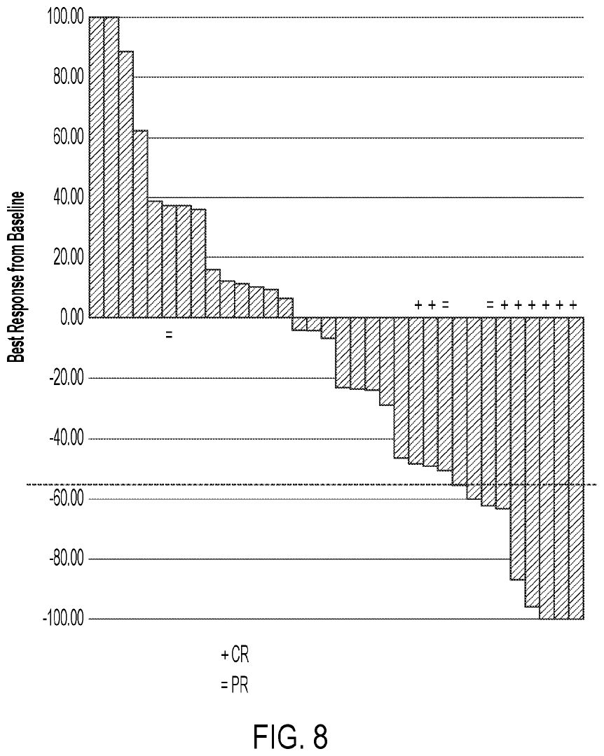

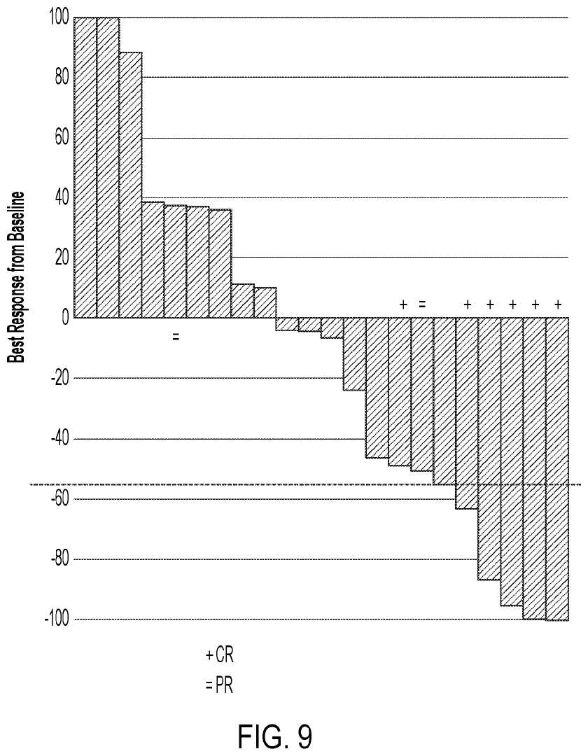

A waterfall plot of the best overall response for each patient for all patients and for alternative dosing patients are shown in FIG. 8 and FIG. 9, respectively. 11 of 34 total patients that had a baseline and on treatment scan have had either a complete response (CR) or a partial response (PR) and 20 showed evidence of tumor size reduction. Of the alternative dose patients, there were 6 CR, and 13 of 22 patients that had a baseline and on treatment scan showed evidence of tumor size reduction. A list of each responder, along with their tumor type, type of best response, starting dose, and dose at the time of response is shown in Table 8. Response rates, including comparisons of patients assigned to a cohort with an initial maximum dose ≤100 vs. >100, for all patients, DLBCL patients, MCL patients, FL patients, and MZL patients are shown in Tables 9-14, respectively. The PR and CR cut-offs were determined according to the Lugano 2014 criteria (Cheson et al., 2014, J Clin Oncol, 32(27): 3059-3067).

TABLE 9
Overview of Responders

| | Best | Starting | Dose at |
|---|---|---|---|
| Tumor Type | Response | Dose | Response |
| MZL | CR | 10 mg | 100 mg |
| DLBCL | CR | 30 mg | 100 mg |
| FL | PR | 30 mg | 100 mg |
| FL | CR | 50 mg* | 100 mg |
| FL | CR | 50 mg* | 100 mg |
| DLBCL | CR | 50 mg* | 100 mg |
| MCL | PR | 50 mg* | 100 mg |
| FL | CR | 50 mg* | 300 mg |
| DLBCL | PR | 50 mg* | 600 mg |
| DLBCL | CR | 50 mg* | 100 mg |
| DLBCL | CR | 15 mg* | 100 mg |

TABLE 10
Response Rate of All Patients

| | All Doses | Doses ≤100 mg | Doses >100 mg |
|---|---|---|---|
| Response | (n = 38) | (n = 21) | (n = 17) |
| ORR | 29% (11) | 43% (9) | 12% (2) |
| CR | 21% (8) | 33% (7) | 6% (1) |
| PR | 8% (3) | 10% (2) | 6% (1) |

TABLE 11
Response Rate of DLBCL Patients

| DLBCL | All Doses | Doses ≤100 mg | Doses >100 mg |
|---|---|---|---|
| Response | (n = 16) | (n = 8) | (n = 8) |
| ORR | 31% (5) | 50% (4) | 13% (1) |
| CR | 25% (4) | 50% (4) | 0% (0) |
| PR | 6% (1) | 0% (0) | 13% (1) |

TABLE 12
Response Rate of MCL Patients

| MCL | All Doses | Doses ≤100 mg | Doses >100 mg |
|---|---|---|---|
| Response | (n = 4) | (n = 3) | (n = 1) |
| ORR | 25% (1) | 33% (1) | 0% (0) |
| CR | 0% (0) | 0% (0) | 0% (0) |
| PR | 25% (1) | 33% (1) | 0% (0) |

TABLE 13
Response Rate of FL Patients

| FL | All Doses | Doses ≤100 mg | Doses >100 mg |
|---|---|---|---|
| Response | (n = 14) | (n = 8) | (n = 7) |
| ORR | 27% (4) | 38% (3) | 14% (1) |
| CR | 20% (3) | 25% (2) | 14% (1) |
| PR | 7% (1) | 13% (1) | 0% (0) |

TABLE 14
Response Rate of MZL Patients

| MZL | All Doses | Doses ≤100 mg | Doses >100 mg |
|---|---|---|---|
| Response | (n = 4) | (n = 2) | (n = 2) |
| ORR | 25% (1) | 50% (1) | 0% (0) |
| CR | 25% (1) | 50% (1) | 0% (0) |
| PR | 0% (0) | 0% (0) | 0% (0) |

### Example 3: Phase I Clinical Trial Update

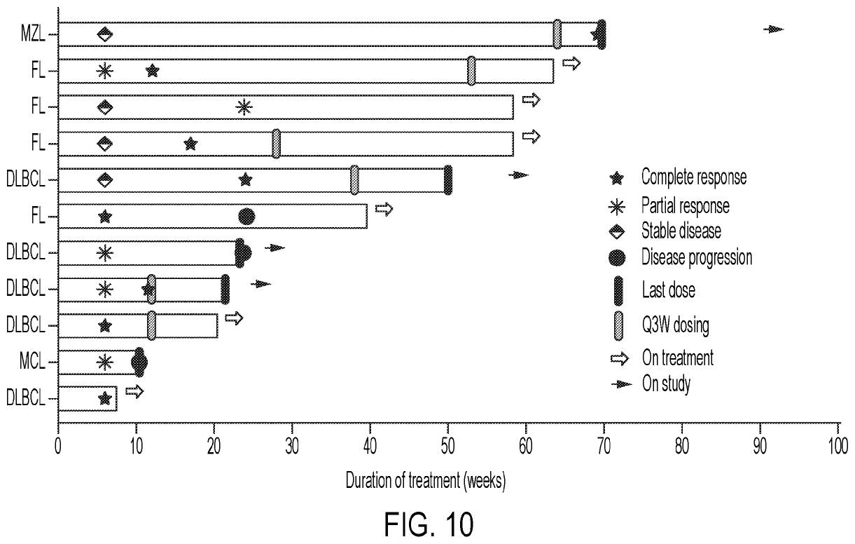

The clinical trial described in Example 1 and Example 2 was continued. No additional patients were evaluated. However, additional data was analyzed. The response timelines for the 11 responding patients previously described in Table 9 are shown in FIG. 10. Response rates (e.g., overall response rate (ORR), complete response (CR), or partial response (PR)), including comparisons of patients assigned to cohorts with various initial maximum doses for all patients, DLBCL patients, MCL patients, FL patients, and MZL patients are shown in Tables 15-19, respectively. The PR and CR cut-offs were determined according to the Lugano 2014 criteria (Cheson et al., 2014, J Clin Oncol, 32(27): 3059-3067).

TABLE 15
Response Rate at Various Assigned Cohort Maximum Doses for All Patients

| | <100 mg | 100 mg | 200 mg | 300 mg | 600 mg | 1000 mg | Total |
|---|---|---|---|---|---|---|---|
| | (n = 11) | (n = 10) | (n = 1) | (n = 7) | (n = 5) | (n = 4) | (N = 38) |
| ORR | 3 (27) | 6 (60) | 0 | 1 (14) | 1 (20) | 0 | 11 (29) |
| CR | 2 (18) | 5 (50) | 0 | 1 (14) | 0 | 0 | 8 (21) |
| PR | 1 (9) | 1 (10) | 0 | 0 | 1 (20) | 0 | 3 (8) |

TABLE 16
Response Rate at Various Assigned Cohort Maximum Doses for DLBCL Patients

| DLBCL | <100 mg | 100 mg | 200 mg | 300 mg | 600 mg | 1000 mg | All doses |
|---|---|---|---|---|---|---|---|
| n (%) | (n = 2) | (n = 6) | (n = 1) | (n = 3) | (n = 3) | (n = 1) | (n = 16) |
| ORR | 1 (50) | 3 (50) | 0 | 0 | 1 (33) | 0 | 5 (31) |
| CR | 1 (50) | 3 (50) | 0 | 0 | 0 | 0 | 4 (25) |
| PR | 0 | 0 | 0 | 0 | 1 (33) | 0 | 1 (6) |

TABLE 17
Response Rate at Various Assigned Cohort Maximum Doses for MCL Patients

| MCL | <100 mg | 100 mg | 200 mg | 300 mg | 600 mg | 1000 mg | All doses |
|---|---|---|---|---|---|---|---|
| n (%) | (n = 2) | (n = 1) | (n = 0) | (n = 0) | (n = 1) | (n = 0) | (n = 4) |
| ORR | 0 | 1 (100) | NA | NA | 0 | NA | 1 (25) |
| CR | 0 | 0 | NA | NA | 0 | NA | 0 |
| PR | 0 | 1 (100) | NA | NA | 0 | NA | 1 (25) |

TABLE 18
Response Rate at Various Assigned Cohort Maximum Doses for FL Patients

| FL | <100 mg | 100 mg | 300 mg | 600 mg | 1000 mg | All doses |
|---|---|---|---|---|---|---|
| n (%) | (n = 5) | (n = 3) | (n = 3) | (n = 1) | (n = 2) | (n = 14) |
| ORR | 1 (20) | 2 (67) | 1 (33) | 0 | 0 | 4 (29) |
| CR | 0 | 2 (67) | 1 (33) | 0 | 0 | 3 (21) |
| PR | 1 (20) | 0 | 0 | 0 | 0 | 1 (7) |

TABLE 19
Response Rate at Various Assigned Cohort Maximum Doses for MZL Patients

| MZL | <100 mg | 100 mg | 300 mg | 600 mg | 1000 mg | All doses |
|---|---|---|---|---|---|---|
| n (%) | (n = 2) | (n = 0) | (n = 1) | (n = 0) | (n = 1) | (n = 4) |
| ORR | 1 (50) | NA | 0 | NA | 0 | 1 (25) |
| CR | 1 (50) | NA | 0 | NA | 0 | 1 (25) |
| PR | 0 | NA | 0 | NA | 0 | 0 |

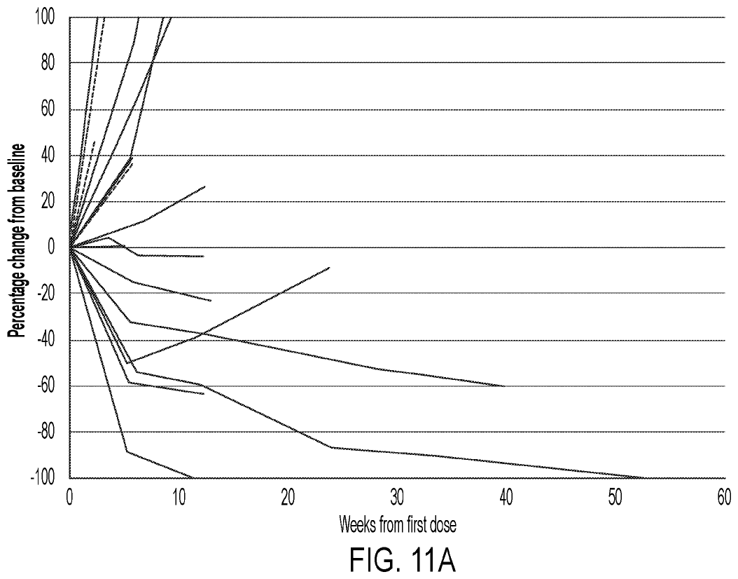

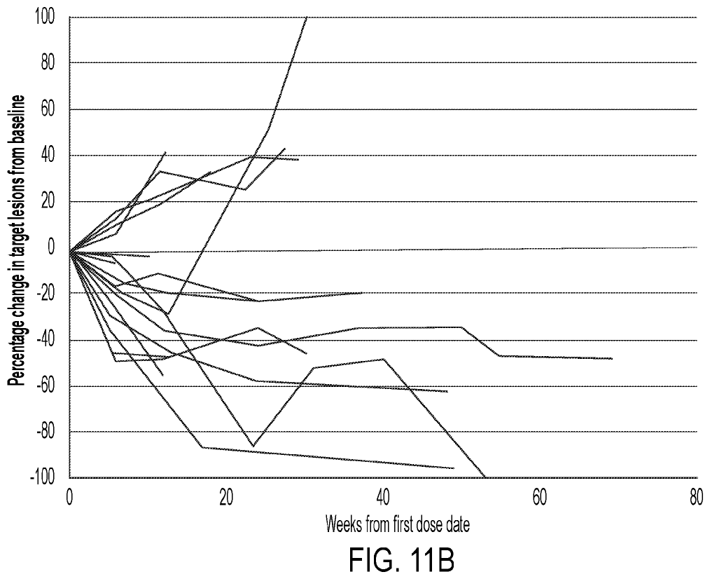

Plots of tumor responses for aggressive and indolent NHL are shown in FIGS. 11A and 11B, respectively. Responses were rapid and durable.

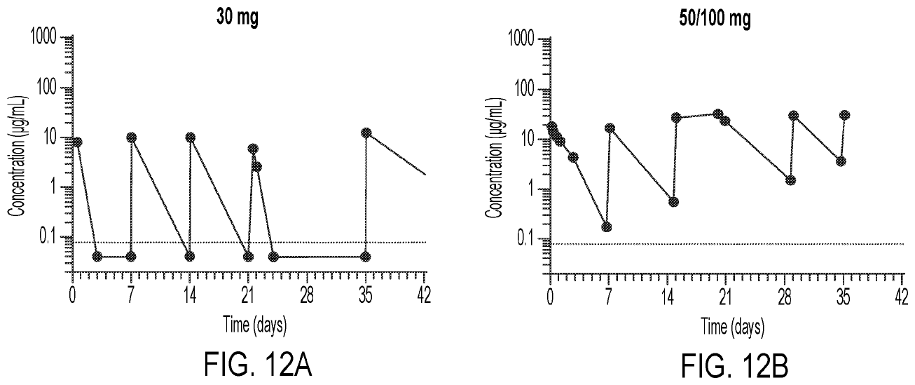

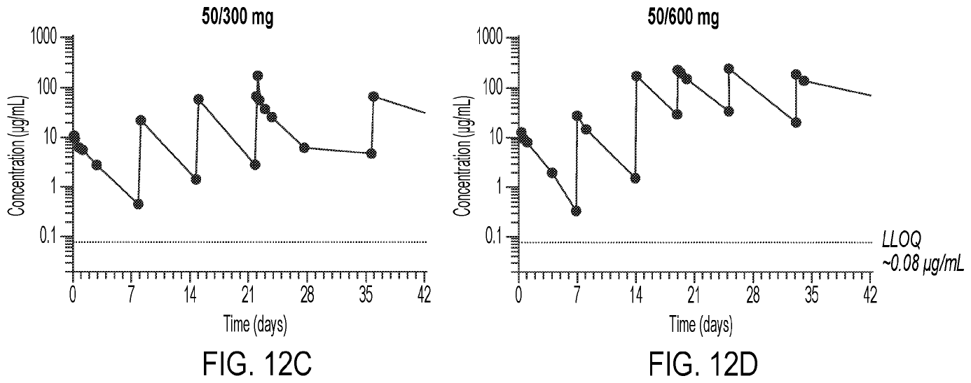

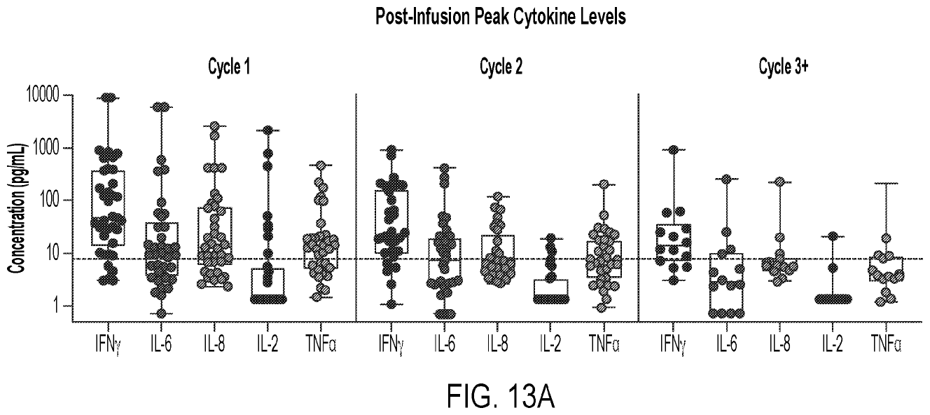

Blood samples were obtained throughout the dosing cycle and the concentration of IGM-2323 was determined using ELISA according to standard methods. The resulting concentrations are shown in FIG. 12 for various cohorts. The preliminary population estimate of half-life (t_{1/2}) is ˜1.5 days. No drug induced antidrug antibodies have been detected.

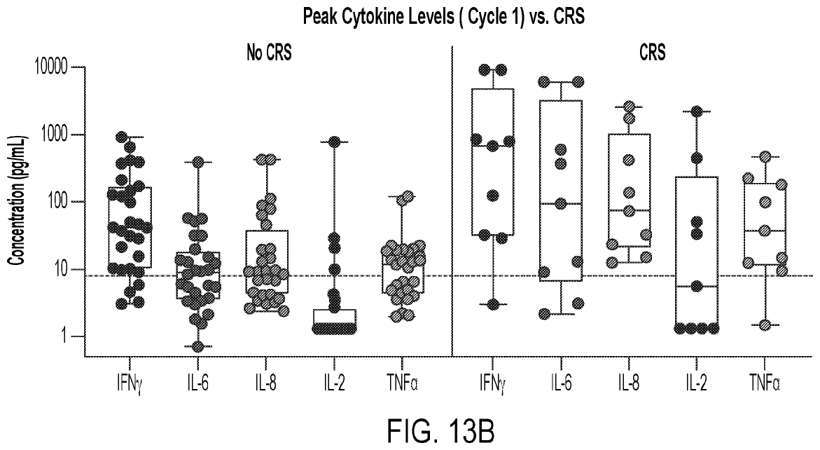

Cytokine levels in frozen plasma were assessed at a central laboratory for 39 patients. FIG. 13A shows the highest concentrations of 5 cytokines obtained during sampling for Cycles 1, 2, and 3+.

As seen in Example 1, IGM-2323 treatment led to transient (˜2-12 hours), repeatable, and IFNγ-dominant immune activity. IGM-2323 was biologically active, as measured by cytokine activity, starting at 10 mg. In most patients treated IFNγ levels were much greater than IL-6, IL-8, IL-2, and TNFα.

Peak cytokine levels in Cycle 1 in patients with any grade cytokine release syndrome (CRS) and no CRS are shown in FIG. 13B. Poly-cytokine response appears to be associated with CRS. The timing of the poly-cytokine increase matched the CRS onset.

Overall, IGM-2323 was active against relapsed/refractory NHL and had a highly favorable safety profile.

### Example 4: B-cell Binding of IGM-2323 in the Presence of Rituximab

NHL patients are commonly treated with rituximab, an anti-CD20 IgG1 monoclonal antibody that binds nearly an identical epitope as IGM-2323 (Klein, C. et al., mAbs, 2013, 22-22 and Bornstein, G., et al., Invest New Drugs, 2010, 28(5): 561-574). The following examples were performed to determine if residual rituximab would inhibit IGM-2323. Rituximab has been reported to remain in a patient's serum for weeks to months after treatment, as reported by Viswabandya A. et al., J Glob Oncol. 2019, 5:1-13 and Muller C. et al., Blood, 2012, 119(14): 3276-3284. Reported rituximab concentrations over time are reproduced in Table 20.

TABLE 20
Rituximab Trough Serum Levels and Cmax
concentrations After 8 14-day cycles treated
with 375 mg/m² rituximab

| Time after | |
|---|---|
| treatment | μg/mL, median (range) |
| 1 week | 172.6 (91.4-286.1) |
| 1 month | 91.4 (39.6-189.1) |
| 2 months | 54.0 (16.1-122.9) |
| 3 months | 30.1 (9.33-82.2) |
| 6 months | 5.1 (0.8-14.7) |
| 9 months | 1.1 (0-2.8) |

Methods for IGM-2323 Binding

An in vitro assay was performed to assess the binding of IGM-2323 in the presence of Rituximab® on the CD20-expressing B-cell lymphoma cell lines: Ramos, CA46, or rituximab-resistant Ramos. The rituximab-resistant Ramos cells were generated as follows. Ramos cells were subjected to multiple rounds of complement dependent cytotoxicity (CDC) using rituximab or an IgM comprising the VH and VL of rituximab and a J chain. Cells were incubated with the antibody and complement. The remaining live cells were allowed to recover and grow in media at 37° C. in a humidified 5% CO₂ incubator for several days. Afterwards, CDC was tested to evaluate resistance. The process was repeated several times on the recovered CDC-resistant pools until the rituximab-resistant cell line was generated. B-cell lymphoma cells (5×10⁶) were washed once with 10 mL 1× PBS (Gibco, cat #20012-043) and resuspended to a cell density of 1×10⁶ cells/mL. 5×10⁴ cells were pre-incubated with rituximab at 2, 10, 20, 100, or 200 μg/mL for 5 minutes at 4° C. 200 μg/mL of IGM-2323 was added to the wells and incubated for 5 minutes or 1 hour at 37° C. Cells were washed twice with 1×PBS and then stained with a mouse anti-human serum albumin (HSA) antibody (Novus Biologicals, cat #NB500-417AF647) for 5 min at 4° C. Cells were washed twice with FACS buffer (1× PBS+2% FBS+2 mM EDTA) and then resuspended in FACS buffer and propidium iodide (BD Biosciences, cat #556463). Cells were analyzed by CytoFLEX LX flow cytometer.

Methods for Rituximab Binding

An in vitro assay was performed to assess the binding of rituximab in the presence of IGM-2323 on Ramos cells. Ramos cells (5×10⁶) were washed once with 10 mL 1× PBS (Gibco, cat #20012-043) and resuspended to a cell density of 1×10⁶ cells/mL. 5×10⁴ cells were pre-incubated with Alexa Fluor 647-labeled rituximab at 2, 10, 20, 100, or 200 μg/mL for 5 minutes at 4° C. 1× PBS or 200 μg/mL of IGM-2323 were added to the wells and incubated for 5 minutes or 1 hour at 37° C. Cells were washed twice with FACS buffer (1× PBS+2% FBS+2 mM EDTA) and then resuspended in FACS buffer and propidium iodide (BD Biosciences, cat #556463). Cells were analyzed by CytoFLEX LX flow cytometer.

Methods for CD20×CD3 IgG Binding

An in vitro assay was performed to assess the binding of an anti-CD20×anti-CD3 IgG antibody (CD20×CD3 IgG) comprising one copy of SEQ ID NO: 63, two copies of SEQ ID NO: 64, and one copy of SEQ ID NO: 65 in the presence of rituximab on Ramos cells. Ramos cells (5×10⁶) were washed once with 10 mL 1× PBS (Gibco, cat #20012-043) and resuspended to a cell density of 1×10⁶ cells/mL. 5×10⁴ cells were pre-incubated with rituximab at 2, 10, 20, 100, or 200 μg/mL for 5 minutes at 4° C. 30 μg/mL of Alexa Fluor 647-labeled CD20×CD3 IgG was added to the wells and incubated for 5 minutes or 1 hour at 37° C. Cells were washed twice with FACS buffer (1× PBS+2% FBS+2 mM EDTA) and then resuspended in FACS buffer and propidium iodide (BD Biosciences, cat #556463). Cells were analyzed by CytoFLEX LX flow cytometer.

Results

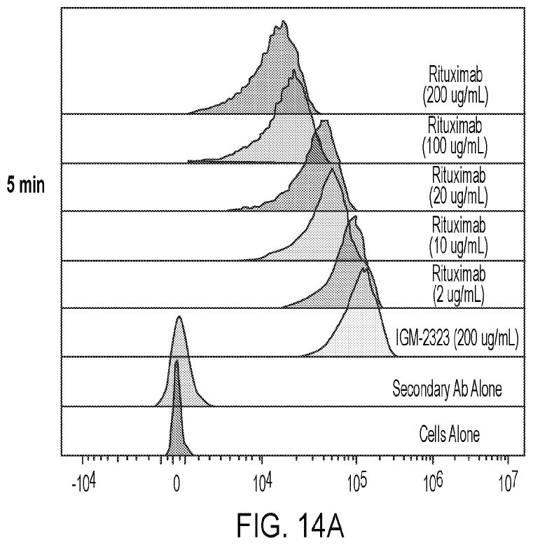

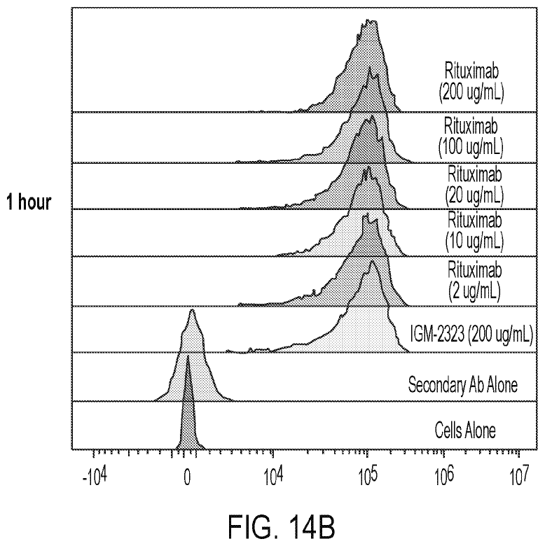

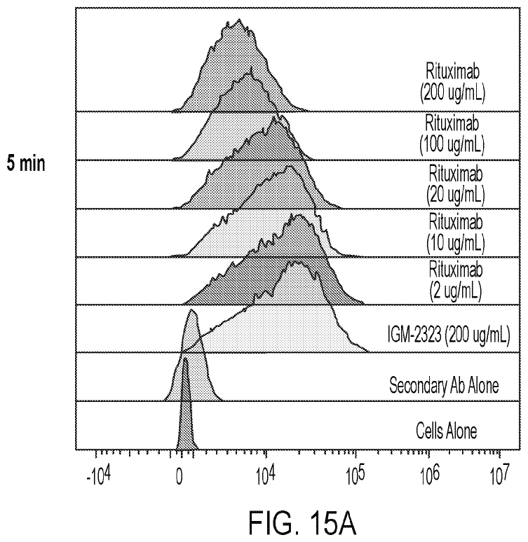

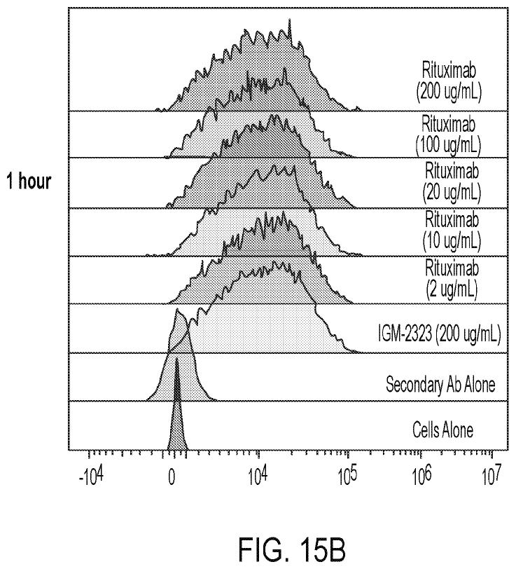

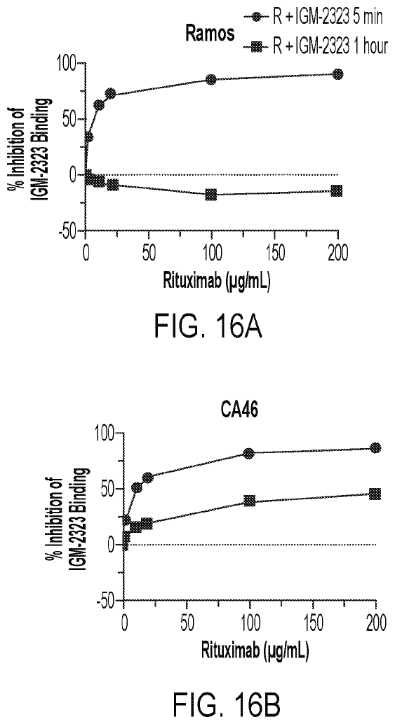

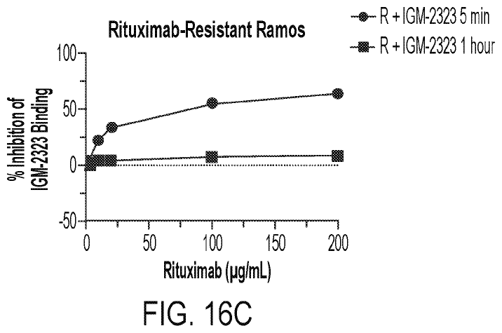

Binding of IGM-2323 to Ramos or rituximab-resistant Ramos cell in the presence of various amounts of rituximab is shown in FIGS. 14A-14B and 15A-15B, respectively. FIGS. 14A and 15A show binding after 5 minutes, and FIGS. 14B and 15B show binding after 1 hour. The percent inhibition for each condition was calculated by dividing the amount of binding at a particular concentration of rituximab by the amount of binding without rituximab and multiplying by 100 then subtracting from 100. The % inhibition of IGM-2323 binding Ramos, CA46, and rituximab-resistant Ramos cells is shown in FIGS. 16A, 16B, and 16C, respectively.

For Ramos cells, longer incubation time (1 hour vs. 5 minutes) of IGM-2323 prevented inhibition by rituximab, whereas for CA46 cells and rituximab-resistant Ramos cells, longer incubation time of IGM-2323 reduced inhibition by rituximab.

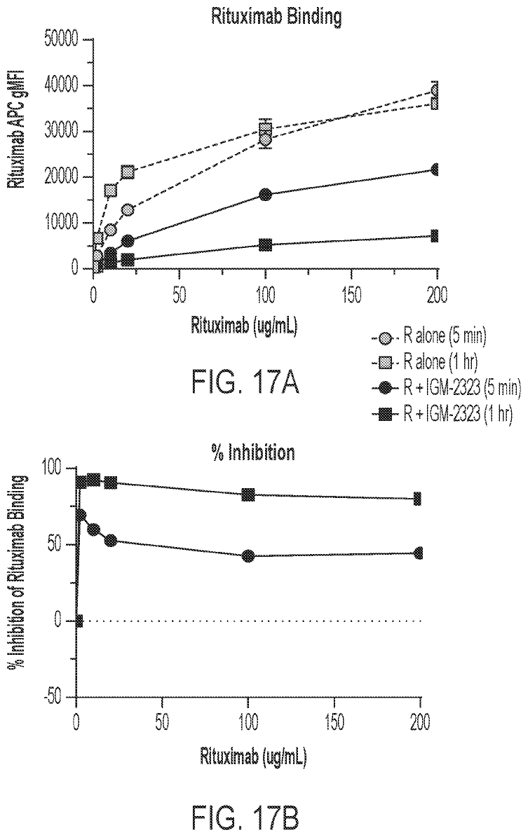

The amount of binding of rituximab with or without the presence of IGM-2323 was also measured and is shown in FIG. 17A. The percent inhibition of rituximab binding in the presence of IGM-2323 is shown in FIG. 17B. Longer incubation time of IGM-2323 decreased rituximab binding.

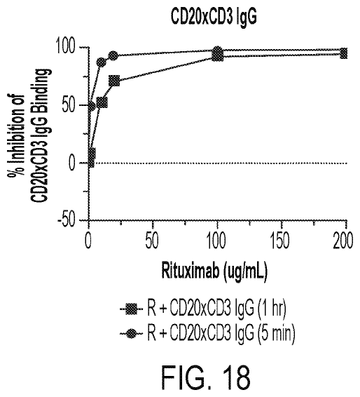

The percent inhibition of CD20×CD3 IgG binding in the presence of rituximab is shown in FIG. 18. Strong rituximab inhibition was still present with longer incubation of CD20×CD3 IgG in Ramos cells.

### Example 5: Complement-Dependent Cytotoxicity Imaging

To determine if the presence of rituximab inhibited complement dependent cytotoxicity (CDC) for IGM-2323, a series of CDC imaging experiments were performed.

1×10⁶ Ramos, CA46, or Rituximab-resistant Ramos cells were centrifuged for 5 min at 1500 rpm at room temperature and washed once in 10 mL RPMI-1640 media. Cells were resuspended in 1 mL media with 2 nM CELLTRACE™ OREGON GREEN™ (Thermo Fisher Scientific) and were incubated for 30 min at 37° C., washed twice in 10 mL media, and resuspended in 1 mL media. Cells were transferred to a 1.5 mL EPPENDORF TUBE®, centrifuged for 5 min at 300×g at room temperature, and resuspended in 120 μL media. 9 μM DRAQ7™ (BioLegend) was then added. Labeled cells were added to an EPPENDORF TUBE® with 4× concentrated rituximab, and incubated for 5 min at 37° C. Then 4× concentrated IGM-2323 was added and incubated for 5 min or 1 hour at 37° C. Finally, 50% Normal Human Serum (Quidel) was added and the 10 μL was loaded on a C-CHIP™ disposable hemacytometer (Neubauer Improved Grid, 100 μm Chamber Depth) (Incyto). The slide was added to a temperature- and CO₂-controlled LIONHEART™ Fx microscope (Biotek) and imaged with 4× objective with GFP and Cy5 filters, LED power of 10, integration time of 250 msec, and gain of 15. Analysis was done using the Biotek cell analysis software (Cell counting) to quantify the percentage of dead cells. The dead cell % was further analyzed with GraphPad Prism using a plateau followed by one phase decay nonlinear regression.

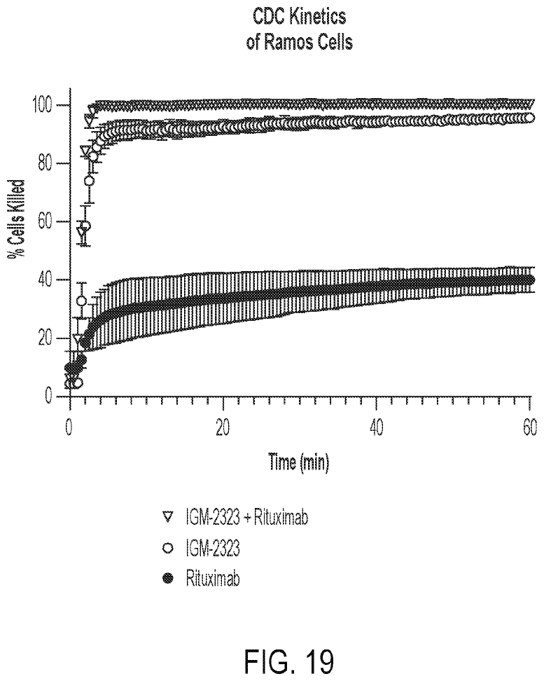

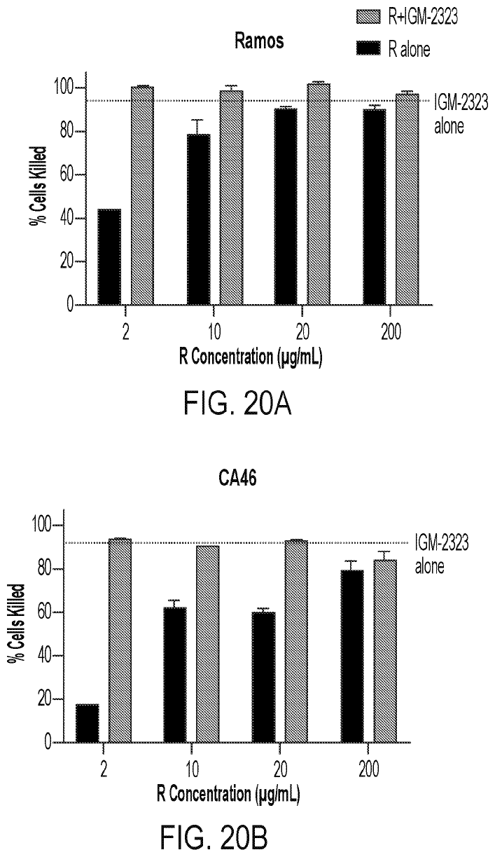

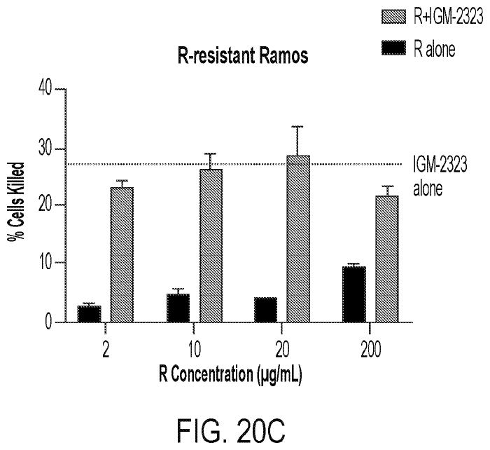

The % cells killed for 200 ng/mL IGM-2323 alone and with 2 ng/mL rituximab and for 2 ng/mL rituximab alone is shown in FIG. 19. At these concentrations, the combination of rituximab and IGM-2323 had greater killing than either molecule alone. The % cells killed for 200 ng/mL IGM-2323 with various concentrations of rituximab and for various concentrations of rituximab alone on Ramos, CA46, and rituximab-resistant Ramos cells is shown in FIGS. 20A-20C, respectively. The combination of IGM-2323 and rituximab enhanced CDC except when rituximab was at 200 ng/mL, where some inhibition of IGM-2323 was present.

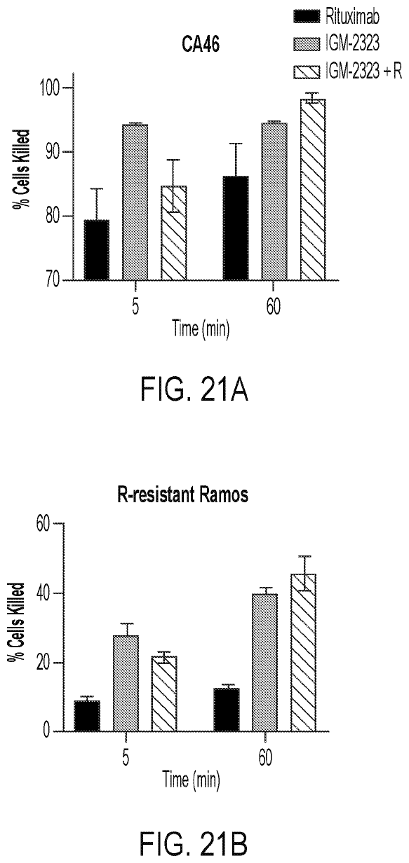

The % cells killed of CA46 and rituximab-resistant cells after 5 or 60 minutes of treatment with 200 ng/mL IGM-2323 alone and with 200 ng/mL rituximab or with 200 ng/mL rituximab alone is shown in FIGS. 21A and 21B, respectively. The longer incubation of the combination of IGM-2323 and rituximab enhanced CDC over either molecule alone.

### Example 6: T Cell Dependent Cellular Cytotoxicity (TDCC) and T Cell Activation Assay (TCA)

To determine if the presence of rituximab inhibited T cell dependent cellular cytotoxicity (TDCC) and T cell activation (TCA) for IGM-2323, a series of TDCC and TCA experiments were performed.

2.5×10⁴ B-cell lymphoma cell lines (Ramos, CA46, and rituximab-resistant Ramos) were co-cultured with 1.25×10 5 primary pan CD3+ T cells at a 5:1 effector to target (E:T) ratio. The B-cell lines were labeled with 3 nM CELLTRACE™ OREGON GREEN (Thermo Fisher Scientific) for 30 min at 37° C., and subsequently washed with RPMI-1640 medium. The pan T cells were CD3 negatively selected T cells isolated from whole blood of healthy donors (Discovery Life Sciences). Cells were incubated in the presence of 4-fold serial dilutions of IGM-2323 from 200 to 0.003052 μg/mL or CD20×CD3 IgG antibody from to 0.000469 μg/mL with 3-fold serial dilution of rituximab from 200 to 2.47 μg/mL in 200 μL of RPMI-1640 medium per well on a 96-well round bottom plate. After 48 hours at 37° C. in a 5% CO₂ incubator, the plates were centrifuged at 4° C. and incubated for 30 minutes at 4° C. with EBIOSCIENCE™ Fixable Viability Dye EFLUOR™ 780 (Thermo Fisher Scientific), ALEXA FLUOR® 700 anti-human CD3 antibody (BioLegend), BRILLIANT VIOLET 421™ anti-human CD4 antibody (BioLegend), BRILLIANT VIOLET 605™ anti-human CD8 (BioLegend) antibody, ALEXA FLUOR® 647 anti-human CD25 antibody (BioLegend), PerCP-Cyanine5.5 anti-human CD69 antibody (BioLegend), and BRILLIANT VIOLET 650™ anti-human CD279 (PD-1) (BioLegend) antibody in FACS buffer. The plates were then centrifuged, and the cells are fixed in 2% paraformaldehyde for 15 minutes at 4° C. Cells were washed in FACS buffer and resuspended in 100 μL of FACS buffer for acquisition on a CytoFLEX LX flow cytometer (Beckman Coulter). Percent killing of target cells and T cell activation were quantified by comparison between treated samples and a non-treatment control.

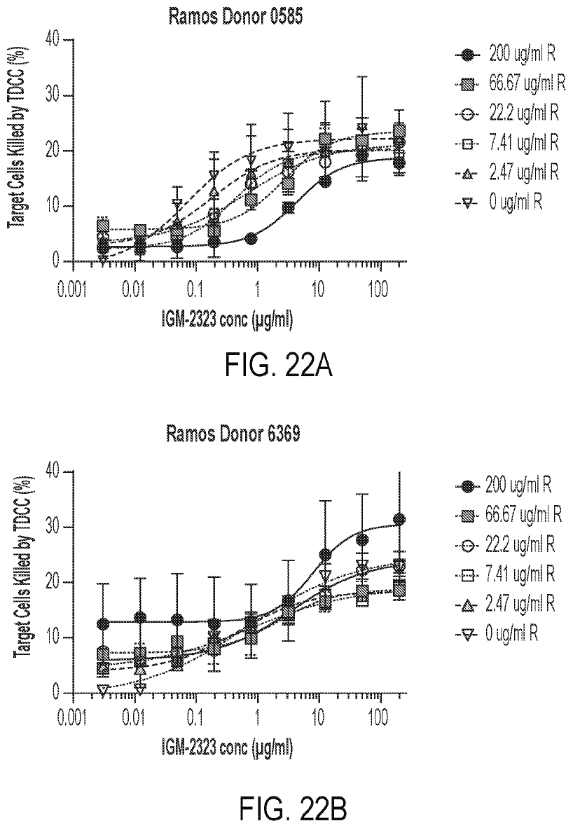

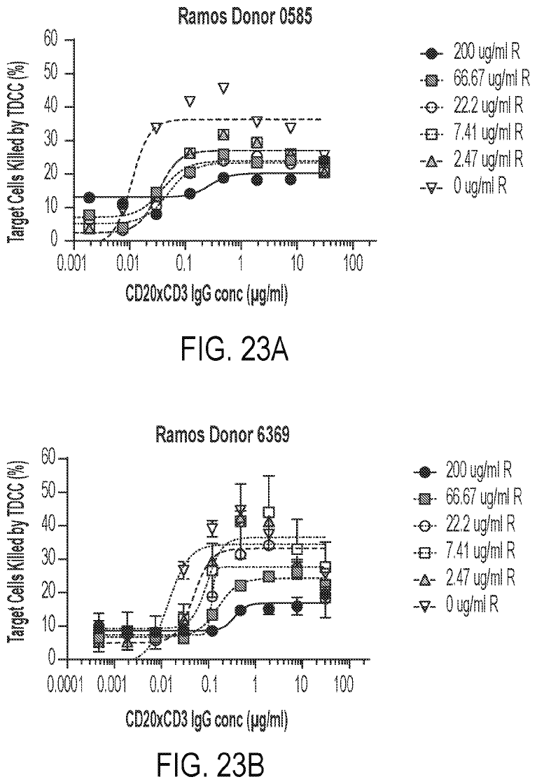

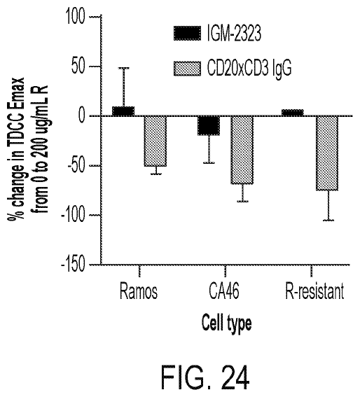

The percent of TDCC killing of Ramos cells for T cell donor 0585 and donor 6369 with various concentration of IGM-2323 and rituximab are shown in FIGS. 22A and 22B, respectively, and the percent of TDCC killing of Ramos cells for T cell donor 0585 and donor 6369 with various concentration of CD20×CD3 IgG and rituximab are shown in FIGS. 23A and 23B, respectively. CD20×CD3 IgG was more inhibited by the presence of rituximab than IGM-2323 on Ramos cells. The percent change in TDCC maximum killing activity (Emax) from 0 to 200 μg/mL rituximab was calculated by determining the Emax at for each assay with 0 μg/mL and 200 μg/mL rituximab, and then calculating the percent change. An average was taken for the two donors. Donor 6369 for the rituximab-resistant Ramos cells with IGM-2323 was excluded from the calculate because there was little to no killing for those conditions. The results are shown in FIG. 24. Only a minor impact to the Emax of IGM-2323 was observed at the Cmax of rituximab (200 ng/mL). In contrast, rituximab pre-treatment resulted in a distinctly lower Emax of CD20×CD3 IgG.

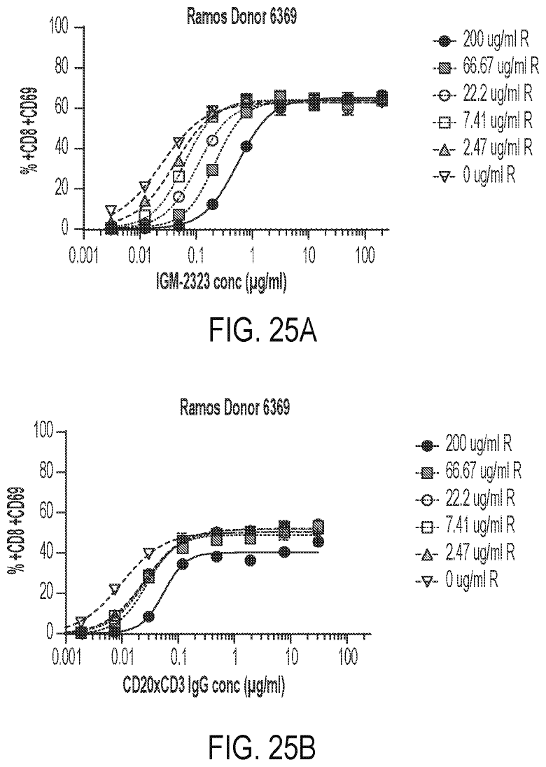

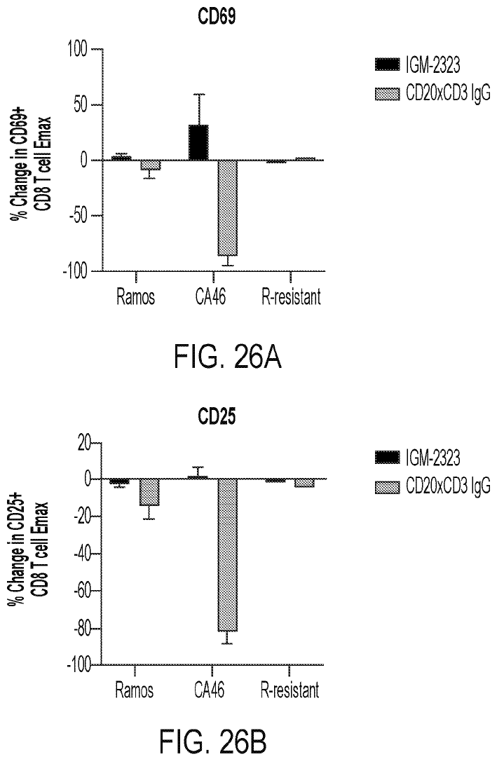

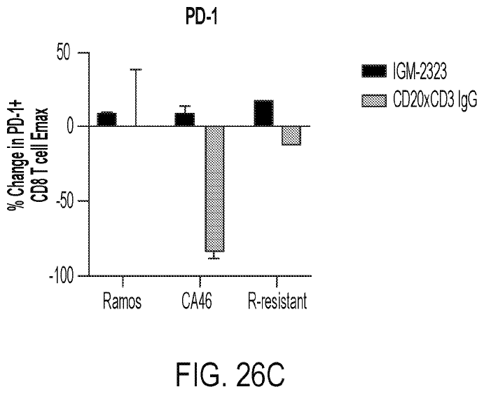

The percent of CD69 positive CD8 T cells, an exemplary TCA assay, for T cell donor 0585 and donor 6369 with various concentration of IGM-2323 and rituximab are shown in FIGS. 25A and 25B, respectively. The percent change in maximum percentage of cells with the activation marker (Emax) from 0 to 200 μg/mL rituximab was calculated by determining the Emax for each assay with 0 μg/mL and 200 μg/mL rituximab, and then calculating the percent change. An average was taken for the two donors. The results for CD69+ CD8 T cells, CD25+ CD8 T cells, and PD-1+ CD8 T cells are shown in FIGS. 26A-26C, respectively.

TABLE 21
Sequences in the Disclosure

| SEQ ID | | |
|---|---|---|
| NO: | Nickname/Source | Sequence |
| 1 | Human IgM Constant | GSASAPTLFPLVSCENSPSDTSSVAVGCLAQDFLPDSITFSWKYKN |
| | region IMGT allele | NSDISSTRGFPSVLRGGKYAATSQVLLPSKDVMQGTDEHVVCKVQH |
| | IGHM*03 (GenBank: | PNGNKEKNVPLPVIAELPPKVSVFVPPRDGFFGNPRKSKLICQATG |
| | pir\|S37768\|) | FSPRQIQVSWLREGKQVGSGVTTDQVQAEAKESGPTTYKVTSTLTI |
| | | KESDWLSQSMFTCRVDHRGLTFQQNASSMCVPDQDTAIRVFAIPPS |
| | | FASIFLTKSTKLTCLVTDLTTYDSVTISWTRQNGEAVKTHTNISES |
| | | HPNATFSAVGEASICEDDWNSGERFTCTVTHTDLPSPLKQTISRPK |
| | | GVALHRPDVYLLPPAREQLNLRESATITCLVTGFSPADVFVQWMQR |
| | | GQPLSPEKYVTSAPMPEPQAPGRYFAHSILTVSEEEWNTGETYTCV |
| | | VAHEALPNRVTERTVDKSTGKPTLYNVSLVMSDTAGTCY |
| 2 | Human IgM Constant | GSASAPTLFPLVSCENSPSDTSSVAVGCLAQDFLPDSITFSWKYKN |
| | region IMGT allele | NSDISSTRGFPSVLRGGKYAATSQVLLPSKDVMQGTDEHVVCKVQH |
| | IGHM*04 (GenBank: | PNGNKEKNVPLPVIAELPPKVSVFVPPRDGFFGNPRKSKLICQATG |
| | sp\|P01871.4\|) | FSPRQIQVSWLREGKQVGSGVTTDQVQAEAKESGPTTYKVTSTLTI |
| | | KESDWLGQSMFTCRVDHRGLTFQQNASSMCVPDQDTAIRVFAIPPS |
| | | FASIFLTKSTKLTCLVTDLTTYDSVTISWTRQNGEAVKTHTNISES |
| | | HPNATFSAVGEASICEDDWNSGERFTCTVTHTDLPSPLKQTISRPK |
| | | GVALHRPDVYLLPPAREQLNLRESATITCLVTGFSPADVFVQWMQR |
| | | GQPLSPEKYVTSAPMPEPQAPGRYFAHSILTVSEEEWNTGETYTCV |
| | | VAHEALPNRVTERTVDKSTGKPTLYNVSLVMSDTAGTCY |
| 3 | Human IgA1 heavy | ASPTSPKVFPLSLCSTQPDGNVVIACLVQGFFPQEPLSVTWSESGQ |
| | chain constant | GVTARNFPPSQDASGDLYTTSSQLTLPATQCLAGKSVTCHVKHYTN |
| | region, e.g., | PSQDVTVPCPVPSTPPTPSPSTPPTPSPSCCHPRLSLHRPALEDLL |
| | amino acids 144 to | LGSEANLTCTLTGLRDASGVTFTWTPSSGKSAVQGPPERDLCGCYS |
| | 496 of GenBank | VSSVLPGCAEPWNHGKTFTCTAAYPESKTPLTATLSKSGNTFRPEV |
| | AIC59035.1 | HLLPPPSEELALNELVTLTCLARGFSPKDVLVRWLQGSQELPREKY |
| | | LTWASRQEPSQGTTTFAVTSILRVAAEDWKKGDTFSCMVGHEALPL |
| | | AFTQKTIDRLAGKPTHVNVSVVMAEVDGTCY |
| 4 | Human IgA2 heavy | ASPTSPKVFPLSLDSTPQDGNVVVACLVQGFFPQEPLSVTWSESGQ |
| | chain constant | NVTARNFPPSQDASGDLYTTSSQLTLPATQCPDGKSVTCHVKHYTN |
| | region, e.g., | SSQDVTVPCRVPPPPPCCHPRLSLHRPALEDLLLGSEANLTCTLTG |
| | amino acids 1 to | LRDASGATFTWTPSSGKSAVQGPPERDLCGCYSVSSVLPGCAQPWN |
| | 340 of GenBank | HGETFTCTAAHPELKTPLTANITKSGNTFRPEVHLLPPPSEELALN |
| | P01877.4 | ELVTLTCLARGFSPKDVLVRWLQGSQELPREKYLTWASRQEPSQGT |
| | | TTYAVTSILRVAAEDWKKGETFSCMVGHEALPLAFTQKTIDRMAGK |
| | | PTHINVSVVMAEADGTCY |
| 5 | Precursor Human | MLLFVLTCLLAVEPAISTKSPIFGPEEVNSVEGNSVSITCYYPPTS |
| | Secretory | VNRHTRKYWCRQGARGGCITLISSEGYVSSKYAGRANLTNFPENGT |
| | Component | FVVNIAQLSQDDSGRYKCGLGINSRGLSFDVSLEVSQGPGLINDTK |
| | | VYTVDLGRTVTINCPFKTENAQKRKSLYKQIGLYPVLVIDSSGYVN |
| | | PNYTGRIRLDIQGTGQLLFSVVINQLRLSDAGQYLCQAGDDSNSNK |
| | | KNADLQVLKPEPELVYEDLRGSVTFHCALGPEVANVAKFLCRQSSG |
| | | ENCDVVVNTLGKRAPAFEGRILLNPQDKDGSFSVVITGLRKEDAGR |
| | | YLCGAHSDGQLQEGSPIQAWQLFVNEESTIPRSPTVVKGVAGGSVA |
| | | VLCPYNRKESKSIKYWCLWEGAQNGRCPLLVDSEGWVKAQYEGRLS |
| | | LLEEPGNGTFTVILNQLTSRDAGFYWCLINGDTLWRTTVEIKIIEG |
| | | EPNLKVPGNVTAVLGETLKVPCHFPCKFSSYEKYWCKWNNTGCQAL |
| | | PSQDEGPSKAFVNCDENSRLVSLTLNLVTRADEGWYWCGVKQGHFY |
| | | GETAAVYVAVEERKAAGSRDVSLAKADAAPDEKVLDSGFREIENKA |
| | | IQDPRLFAEEKAVADTRDQADGSRASVDSGSSEEQGGSSRALVSTL |
| | | VPLGLVLAVGAVAVGVARARHRKNVDRVSIRSYRTDISMSDFENSR |
| | | EFGANDNMGASSITQETSLGGKEEFVATTESTTETKEPKKAKRSSK |
| | | EEAEMAYKDFLLQSSTVAAEAQDGPQEA |
| 6 | Precursor Human J | MKNHLLFWGVLAVFIKAVHVKAQEDERIVLVDNKCKCARITSRIIR |
| | Chain | SSEDPNEDIVERNIRIIVPLNNRENISDPTSPLRTRFVYHLSDLCK |
| | | KCDPTEVELDNQIVTATQSNICDEDSATETCYTYDRNKCYTAVVPL |
| | | VYGGETKMVETALTPDACYPD |
| 7 | Mature Human J | QEDERIVLVDNKCKCARITSRIIRSSEDPNEDIVERNIRIIVPLNN |
| | Chain | RENISDPTSPLRTRFVYHLSDLCKKCDPTEVELDNQIVTATQSNIC |
| | | DEDSATETCYTYDRNKCYTAVVPLVYGGETKMVETALTPDACYPD |
| 8 | J Chain Y102A | QEDERIVLVDNKCKCARITSRIIRSSEDPNEDIVERNIRIIVPLNN |
| | mutation | RENISDPTSPLRTRFVYHLSDLCKKCDPTEVELDNQIVTATQSNIC |
| | | DEDSATETCATYDRNKCYTAVVPLVYGGETKMVETALTPDACYPD |
| 9 | ″5″ Peptide | GGGGS |
| | linker | |
| 10 | ″10″ Peptide | GGGGSGGGGS |
| | linker | |
| 11 | ″15″ Peptide | GGGGSGGGGSGGGGS |
| | linker | |
| 12 | ″20″ Peptide | GGGGSGGGGGGGGSGGGGS |
| | linker | |
| 13 | ″25″ Peptide | GGGGSGGGGSGGGGSGGGGSGGGGS |
| | Linker | |
| 14 | WO2015095392 | EVQLVESGGGLVQPKGSLKLSCAASGFTENTYAMNWVRQAPGKGLE |
| | Anti-CD-3 VH | WVARIRSKYNNYATYYADSVKDRFTISRDDSQSILYLQMNNLKTED |
| | | TAMYYCVRHGNFGNSYVSWFAYWGQGTLVTVSS |
| 15 | WO2015095392-Anti- | QAVVTQESALTTSPGETVTLTCRSSTGAVTTSNYANWVQEKPDHLF |
| | CD-3 VL | TGLIGGTNKRAPGVPARFSGSLIGDKAALTITGAQTEDEAIYFCAL |
| | | WYSNLWVFGGGTKLTVL |
| 16 | US5834597A-Anti- | QVQLVQSGAEVKKPGASVKVSCKASGYTFISYTMHWVRQAPGQGLE |
| | CD-3 VH | WMGYINPRSGYTHYNQKLKDKATLTADKSASTAYMELSSLRSEDTA |
| | | VYYCARSAYYDYDGFAYWGQGTLVTVSS |
| 17 | US5834597A-Anti- | DIQMTQSPSSLSASVGDRVTITCSASSSVSYMNWYQQKPGKAPKRL |
| | CD-3 VL | IYDTSKLASGVPSRFSGSGSGTDFTLTISSLQPEDFATYYCQQWSS |
| | | NPPTFGGGTKVEIK |
| 18 | AA WO2018208864- | EVQLLESGGGLVQPGGSLRLSCAASGFTFDTYAMNWVRQAPGKGLE |
| | Anti-CD-3 VH | WVARIRSKYNNYATYYADSVKDRFTISRDDSKSTLYLQMESLRAED |
| | | TAVYYCVRHANFGAGYVSWFAHWGQGTLVTVSS |
| 19 | AA WO2018208864- | QTVVTQEPSLSVSPGGTVTLTCGSSTGAVTTSNYANWVQQTPGQAP |
| | Anti-CD-3 VL | RGLIGGTDKRAPGVPDRFSGSLLGDKAALTITGAQAEDEADYYCAL |
| | | WYSNHWVFGGGTKLTVL |
| 20 | AB WO2018208864- | EVQLLESGGGLVQPGGSLRLSCAASGFTFDTYAMNWVRQAPGKGLE |
| | Anti-CD-3 VH | WVARIRSKYNNYATYYADSVKDRFTISRDDSKSTLYLQMESLRAED |
| | | TAVYYCVRHANFGAGYVSWFAHWGQGTLVTVSS |
| 21 | AB WO2018208864- | QTVVTQEPSLSVSPGGTVTLTCGSSTGAVTTSNYANWVQQTPGQAP |
| | Anti-CD-3 VL | RGLIGGTDKRAPGVPDRFSGSLLGDKAALTITGAQAEDEADYYCAL |
| | | WYSDLWVFGGGTKLTVL |
| 22 | AC WO2018208864- | QVQLVQSGAEVKKPGASVKVSCKASGFNIKDYYMHWVRQAPGQRLE |
| | Anti-CD-3 VH | WMGWIDLENANTIYDAKFQGRVTITRDTSASTAYMELSSLRSEDTA |
| | | VYYCARDAYGRYFYDVWGQGTLVTVSS |
| 23 | AC WO2018208864- | DIVMTQSPDSLAVSLGERATINCKSSQSLLNARTGKNYLAWYQQKP |
| | Anti-CD-3 VL | GQPPKLLIYWASTRESGVPDRFSGSGSGTDFTLTISSLQAEDVAVY |
| | | YCKQSYSRRTFGGGTKVEIK |
| 24 | AD WO2018208864- | QVQLVQSGAEVKKPGASVKVSCKASGFNIKDYYMHWVRQAPGQRLE |
| | Anti-CD-3 VH | WIGWIDLENANTVYDAKFQGRVTITRDTSASTAYMELSSLRSEDTA |
| | | VYYCARDAYGRYFYDVWGQGTLVTVSS |
| 25 | AD WO2018208864- | DIVMTQSPDSLAVSLGERATINCKSSQSLLNARTGKNYLAWYQQKP |
| | Anti-CD-3 VL | GQPPKLLIYWASTRESGVPDRFSGSGSGTDFTLTISSLQAEDVAVY |
| | | YCKQSYFRRTFGGGTKVEIK |
| 26 | AE WO2018208864- | QVQLVQSGAEVKKPGASVKVSCKASGFNIKDYYMHWVRQAPGQRLE |
| | Anti-CD-3 VH | WIGWIDLENANTVYDAKFQGRVTITRDTSASTAYMELSSLRSEDTA |
| | | VYYCARDAYGQYFYDVWGQGTLVTVSS |
| 27 | AE WO2018208864- | DIVMTQSPDSLAVSLGERATINCKSSQSLLNARTGKNYLAWYQQKP |
| | Anti-CD-3 VL | GQPPKLLIYWASTRESGVPDRFSGSGSGTDFTLTISSLQAEDVAVY |
| | | YCTQSYFRRTFGGGTKVEIK |
| 28 | 1.5.3 VH | EVQLVQSGAEVKKPGESLKISCKGSGYSFTSYWIGWVRQMPGKGLE |
| | | WMGIIYPGDSDTRYSPSFQGQVTISADKSITTAYLQWSSLKASDTA |
| | | MYYCARHPSYGSGSPNFDYWGQGTLVTVSS |
| 29 | 1.5.3 VL | DIVMTQTPLSSPVTLGQPASISCRSSQSLVYSDGNTYLSWLQQRPG |
| | | QPPRLLIYKISNRFSGVPDRFSGSGAGTDFTLKISRVEAEDVGVYY |
| | | CVQATQFPLTFGGGTKVEIK |
| 30 | HSA | DAHKSEVAHRFKDLGEENFKALVLIAFAQYLQQCPFEDHVKLVNEV |
| | | TEFAKTCVADESAENCDKSLHTLFGDKLCTVATLRETYGEMADCCA |
| | | KQEPERNECFLQHKDDNPNLPRLVRPEVDVMCTAFHDNEETFLKKY |
| | | LYEIARRHPYFYAPELLFFAKRYKAAFTECCQAADKAACLLPKLDE |
| | | LRDEGKASSAKQRLKCASLQKFGERAFKAWAVARLSQRFPKAEFAE |
| | | VSKLVTDLTKVHTECCHGDLLECADDRADLAKYICENQDSISSKLK |
| | | ECCEKPLLEKSHCIAEVENDEMPADLPSLAADFVESKDVCKNYAEA |
| | | KDVFLGMFLYEYARRHPDYSVVLLLRLAKTYETTLEKCCAAADPHE |
| | | CYAKVFDEFKPLVEEPQNLIKQNCELFKQLGEYKFQNALLVRYTKK |
| | | VPQVSTPTLVEVSRNLGKVGSKCCKHPEAKRMPCAEDYLSVVLNQL |
| | | CVLHEKTPVSDRVTKCCTESLVNRRPCFSALEVDETYVPKEFNAET |
| | | FTFHADICTLSEKERQIKKQTALVELVKHKPKATKEQLKAVMDDFA |
| | | AFVEKCCKADDKETCFAEEGKKLVAASQAALGL |
| 31 | V scFv | QVQLVQSGAEVKKPGASVKVSCKASGYTFISYTMHWVRQAPGQGLE |
| | | WMGYINPRSGYTHYNQKLKDKATLTADKSASTAYMELSSLRSEDTA |
| | | VYYCARSAYYDYDGFAYWGQGTLVTVSSGGGGSGGGGSGGGGSDIQ |
| | | MTQSPSSLSASVGDRVTITCSASSSVSYMNWYQQKPGKAPKRLIYD |
| | | TSKLASGVPSRFSGSGSGTDFTLTISSLQPEDFATYYCQQWSSNPP |
| | | TFGGGTKVEIK |
| 32 | V15J (″WT″, or | QVQLVQSGAEVKKPGASVKVSCKASGYTFISYTMHWVRQAPGQGLE |
| | wild-type) | WMGYINPRSGYTHYNQKLKDKATLTADKSASTAYMELSSLRSEDTA |
| | | VYYCARSAYYDYDGFAYWGQGTLVTVSSGGGGSGGGGSGGGGSDIQ |
| | | MTQSPSSLSASVGDRVTITCSASSSVSYMNWYQQKPGKAPKRLIYD |
| | | TSKLASGVPSRFSGSGSGTDFTLTISSLQPEDFATYYCQQWSSNPP |
| | | TFGGGTKVEIKGGGGSGGGGSGGGGSQEDERIVLVDNKCKCARITS |
| | | RIIRSSEDPNEDIVERNIRIIVPLNNRENISDPTSPLRTRFVYHLS |
| | | DLCKKCDPTEVELDNQIVTATQSNICDEDSATETCYTYDRNKCYTA |
| | | VVPLVYGGETKMVETALTPDACYPD |
| 33 | V15J* | QVQLVQSGAEVKKPGASVKVSCKASGYTFISYTMHWVRQAPGQGLE |
| | | WMGYINPRSGYTHYNQKLKDKATLTADKSASTAYMELSSLRSEDTA |
| | | VYYCARSAYYDYDGFAYWGQGTLVTVSSGGGGSGGGGSGGGGSDIQ |
| | | MTQSPSSLSASVGDRVTITCSASSSVSYMNWYQQKPGKAPKRLIYD |
| | | TSKLASGVPSRFSGSGSGTDFTLTISSLQPEDFATYYCQQWSSNPP |
| | | TFGGGTKVEIKGGGGSGGGGSGGGGSQEDERIVLVDNKCKCARITS |
| | | RIIRSSEDPNEDIVERNIRIIVPLNNRENISDPTSPLRTRFVYHLS |
| | | DLCKKCDPTEVELDNQIVTATQSNICDEDSATETCATYDRNKCYTA |
| | | VVPLVYGGETKMVETALTPDACYPD |
| 34 | V15J15HSA | QVQLVQSGAEVKKPGASVKVSCKASGYTFISYTMHWVRQAPGQGLE |
| | | WMGYINPRSGYTHYNQKLKDKATLTADKSASTAYMELSSLRSEDTA |
| | | VYYCARSAYYDYDGFAYWGQGTLVTVSSGGGGGGGGSGGGGSDIQ |
| | | MTQSPSSLSASVGDRVTITCSASSSVSYMNWYQQKPGKAPKRLIYD |
| | | TSKLASGVPSRFSGSGSGTDFTLTISSLQPEDFATYYCQQWSSNPP |
| | | TFGGGTKVEIKGGGGSGGGGSGGGGSQEDERIVLVDNKCKCARITS |
| | | RIIRSSEDPNEDIVERNIRIIVPLNNRENISDPTSPLRTRFVYHLS |
| | | DLCKKCDPTEVELDNQIVTATQSNICDEDSATETCYTYDRNKCYTA |
| | | VVPLVYGGETKMVETALTPDACYPDGGGGSGGGGSGGGGSDAHKSE |
| | | VAHRFKDLGEENFKALVLIAFAQYLQQCPFEDHVKLVNEVTEFAKT |
| | | CVADESAENCDKSLHTLFGDKLCTVATLRETYGEMADCCAKQEPER |
| | | NECFLQHKDDNPNLPRLVRPEVDVMCTAFHDNEETFLKKYLYEIAR |
| | | RHPYFYAPELLFFAKRYKAAFTECCQAADKAACLLPKLDELRDEGK |
| | | ASSAKQRLKCASLQKFGERAFKAWAVARLSQRFPKAEFAEVSKLVT |
| | | DLTKVHTECCHGDLLECADDRADLAKYICENQDSISSKLKECCEKP |
| | | LLEKSHCIAEVENDEMPADLPSLAADFVESKDVCKNYAEAKDVFLG |
| | | MFLYEYARRHPDYSVVLLLRLAKTYETTLEKCCAAADPHECYAKVF |
| | | DEFKPLVEEPQNLIKQNCELFKQLGEYKFQNALLVRYTKKVPQVST |
| | | PTLVEVSRNLGKVGSKCCKHPEAKRMPCAEDYLSVVLNQLCVLHEK |
| | | TPVSDRVTKCCTESLVNRRPCFSALEVDETYVPKEFNAETFTFHAD |
| | | ICTLSEKERQIKKQTALVELVKHKPKATKEQLKAVMDDFAAFVEKC |
| | | CKADDKETCFAEEGKKLVAASQAALGL |
| 35 | AA scFv | EVQLLESGGGLVQPGGSLRLSCAASGFTFDTYAMNWVRQAPGKGLE |
| | | WVARIRSKYNNYATYYADSVKDRFTISRDDSKSTLYLQMESLRAED |
| | | TAVYYCVRHANFGAGYVSWFAHWGQGTLVTVSSGGGGSGGGGSGGG |
| | | GSQTVVTQEPSLSVSPGGTVTLTCGSSTGAVTTSNYANWVQQTPGQ |
| | | APRGLIGGTDKRAPGVPDRFSGSLLGDKAALTITGAQAEDEADYYC |
| | | ALWYSNHWVFGGGTKLTVL |
| 36 | AA15J | EVQLLESGGGLVQPGGSLRLSCAASGFTFDTYAMNWVRQAPGKGLE |
| | | WVARIRSKYNNYATYYADSVKDRFTISRDDSKSTLYLQMESLRAED |
| | | TAVYYCVRHANFGAGYVSWFAHWGQGTLVTVSSGGGGSGGGGSGGG |
| | | GSQTVVTQEPSLSVSPGGTVTLTCGSSTGAVTTSNYANWVQQTPGQ |
| | | APRGLIGGTDKRAPGVPDRFSGSLLGDKAALTITGAQAEDEADYYC |
| | | ALWYSNHWVFGGGTKLTVLGGGGSGGGGSGGGGSQEDERIVLVDNK |
| | | CKCARITSRIIRSSEDPNEDIVERNIRIIVPLNNRENISDPTSPLR |
| | | TRFVYHLSDLCKKCDPTEVELDNQIVTATQSNICDEDSATETCYTY |
| | | DRNKCYTAVVPLVYGGETKMVETALTPDACYPD |
| 37 | AA15J* | EVQLLESGGGLVQPGGSLRLSCAASGFTFDTYAMNWVRQAPGKGLE |
| | | WVARIRSKYNNYATYYADSVKDRFTISRDDSKSTLYLQMESLRAED |
| | | TAVYYCVRHANFGAGYVSWFAHWGQGTLVTVSSGGGGSGGGGSGGG |
| | | GSQTVVTQEPSLSVSPGGTVTLTCGSSTGAVTTSNYANWVQQTPGQ |
| | | APRGLIGGTDKRAPGVPDRFSGSLLGDKAALTITGAQAEDEADYYC |
| | | ALWYSNHWVFGGGTKLTVLGGGGSGGGGSGGGGSQEDERIVLVDNK |
| | | CKCARITSRIIRSSEDPNEDIVERNIRIIVPLNNRENISDPTSPLR |
| | | TRFVYHLSDLCKKCDPTEVELDNQIVTATQSNICDEDSATETCATY |
| | | DRNKCYTAVVPLVYGGETKMVETALTPDACYPD |
| 38 | AA15 J15HSA | EVQLLESGGGLVQPGGSLRLSCAASGFTFDTYAMNWVRQAPGKGLE |
| | | WVARIRSKYNNYATYYADSVKDRFTISRDDSKSTLYLQMESLRAED |
| | | TAVYYCVRHANFGAGYVSWFAHWGQGTLVTVSSGGGGSGGGGSGGG |
| | | GSQTVVTQEPSLSVSPGGTVTLTCGSSTGAVTTSNYANWVQQTPGQ |
| | | APRGLIGGTDKRAPGVPDRFSGSLLGDKAALTITGAQAEDEADYYC |
| | | ALWYSNHWVFGGGTKLTVLGGGGSGGGGSGGGGSQEDERIVLVDNK |
| | | CKCARITSRIIRSSEDPNEDIVERNIRIIVPLNNRENISDPTSPLR |
| | | TRFVYHLSDLCKKCDPTEVELDNQIVTATQSNICDEDSATETCYTY |
| | | DRNKCYTAVVPLVYGGETKMVETALTPDACYPDGGGGSGGGGSGGG |
| | | GSDAHKSEVAHRFKDLGEENFKALVLIAFAQYLQQCPFEDHVKLVN |
| | | EVTEFAKTCVADESAENCDKSLHTLFGDKLCTVATLRETYGEMADC |
| | | CAKQEPERNECFLQHKDDNPNLPRLVRPEVDVMCTAFHDNEETFLK |
| | | KYLYEIARRHPYFYAPELLFFAKRYKAAFTECCQAADKAACLLPKL |
| | | DELRDEGKASSAKQRLKCASLQKFGERAFKAWAVARLSQRFPKAEF |
| | | AEVSKLVTDLTKVHTECCHGDLLECADDRADLAKYICENQDSISSK |
| | | LKECCEKPLLEKSHCIAEVENDEMPADLPSLAADFVESKDVCKNYA |
| | | EAKDVFLGMFLYEYARRHPDYSVVLLLRLAKTYETTLEKCCAAADP |
| | | HECYAKVFDEFKPLVEEPQNLIKQNCELFKQLGEYKFQNALLVRYT |
| | | KKVPQVSTPTLVEVSRNLGKVGSKCCKHPEAKRMPCAEDYLSVVLN |
| | | QLCVLHEKTPVSDRVTKCCTESLVNRRPCFSALEVDETYVPKEFNA |
| | | ETFTFHADICTLSEKERQIKKQTALVELVKHKPKATKEQLKAVMDD |
| | | FAAFVEKCCKADDKETCFAEEGKKLVAASQAALGL |
| 39 | AB scFv | EVQLLESGGGLVQPGGSLRLSCAASGFTFDTYAMNWVRQAPGKGLE |
| | | WVARIRSKYNNYATYYADSVKDRFTISRDDSKSTLYLQMESLRAED |
| | | TAVYYCVRHANFGAGYVSWFAHWGQGTLVTVSSGGGGSGGGGSGGG |
| | | GSQTVVTQEPSLSVSPGGTVTLTCGSSTGAVTTSNYANWVQQTPGQ |
| | | APRGLIGGTDKRAPGVPDRFSGSLLGDKAALTITGAQAEDEADYYC |
| | | ALWYSDLWVFGGGTKLTVL |
| 40 | AB15J | EVQLLESGGGLVQPGGSLRLSCAASGFTFDTYAMNWVRQAPGKGLE |
| | | WVARIRSKYNNYATYYADSVKDRFTISRDDSKSTLYLQMESLRAED |
| | | TAVYYCVRHANFGAGYVSWFAHWGQGTLVTVSSGGGGSGGGGSGGG |
| | | GSQTVVTQEPSLSVSPGGTVTLTCGSSTGAVTTSNYANWVQQTPGQ |
| | | APRGLIGGTDKRAPGVPDRFSGSLLGDKAALTITGAQAEDEADYYC |
| | | ALWYSDLWVFGGGTKLTVLGGGGSGGGGSGGGGSQEDERIVLVDNK |
| | | CKCARITSRIIRSSEDPNEDIVERNIRIIVPLNNRENISDPTSPLR |
| | | TRFVYHLSDLCKKCDPTEVELDNQIVTATQSNICDEDSATETCYTY |
| | | DRNKCYTAVVPLVYGGETKMVETALTPDACYPD |
| 41 | AB15J* | EVQLLESGGGLVQPGGSLRLSCAASGFTFDTYAMNWVRQAPGKGLE |
| | | WVARIRSKYNNYATYYADSVKDRFTISRDDSKSTLYLQMESLRAED |
| | | TAVYYCVRHANFGAGYVSWFAHWGQGTLVTVSSGGGGSGGGGSGGG |
| | | GSQTVVTQEPSLSVSPGGTVTLTCGSSTGAVTTSNYANWVQQTPGQ |
| | | APRGLIGGTDKRAPGVPDRFSGSLLGDKAALTITGAQAEDEADYYC |
| | | ALWYSDLWVFGGGTKLTVLGGGGSGGGGSGGGGSQEDERIVLVDNK |
| | | CKCARITSRIIRSSEDPNEDIVERNIRIIVPLNNRENISDPTSPLR |
| | | TRFVYHLSDLCKKCDPTEVELDNQIVTATQSNICDEDSATETCATY |
| | | DRNKCYTAVVPLVYGGETKMVETALTPDACYPD |
| 42 | AB15J15HSA | EVQLLESGGGLVQPGGSLRLSCAASGFTFDTYAMNWVRQAPGKGLE |
| | | WVARIRSKYNNYATYYADSVKDRFTISRDDSKSTLYLQMESLRAED |
| | | TAVYYCVRHANFGAGYVSWFAHWGQGTLVTVSSGGGGSGGGGSGGG |
| | | GSQTVVTQEPSLSVSPGGTVTLTCGSSTGAVTTSNYANWVQQTPGQ |
| | | APRGLIGGTDKRAPGVPDRFSGSLLGDKAALTITGAQAEDEADYYC |
| | | ALWYSDLWVFGGGTKLTVLGGGGSGGGGSGGGGSQEDERIVLVDNK |
| | | CKCARITSRIIRSSEDPNEDIVERNIRIIVPLNNRENISDPTSPLR |
| | | TRFVYHLSDLCKKCDPTEVELDNQIVTATQSNICDEDSATETCYTY |
| | | DRNKCYTAVVPLVYGGETKMVETALTPDACYPDGGGGSGGGGSGGG |
| | | GSDAHKSEVAHRFKDLGEENFKALVLIAFAQYLQQCPFEDHVKLVN |
| | | EVTEFAKTCVADESAENCDKSLHTLFGDKLCTVATLRETYGEMADC |
| | | CAKQEPERNECFLQHKDDNPNLPRLVRPEVDVMCTAFHDNEETFLK |
| | | KYLYEIARRHPYFYAPELLFFAKRYKAAFTECCQAADKAACLLPKL |
| | | DELRDEGKASSAKQRLKCASLQKFGERAFKAWAVARLSQRFPKAEF |
| | | AEVSKLVTDLTKVHTECCHGDLLECADDRADLAKYICENQDSISSK |
| | | LKECCEKPLLEKSHCIAEVENDEMPADLPSLAADFVESKDVCKNYA |
| | | EAKDVFLGMFLYEYARRHPDYSVVLLLRLAKTYETTLEKCCAAADP |
| | | HECYAKVFDEFKPLVEEPQNLIKQNCELFKQLGEYKFQNALLVRYT |
| | | KKVPQVSTPTLVEVSRNLGKVGSKCCKHPEAKRMPCAEDYLSVVLN |
| | | QLCVLHEKTPVSDRVTKCCTESLVNRRPCFSALEVDETYVPKEFNA |
| | | ETFTFHADICTLSEKERQIKKQTALVELVKHKPKATKEQLKAVMDD |
| | | FAAFVEKCCKADDKETCFAEEGKKLVAASQAALGL |
| 43 | AC scFv | QVQLVQSGAEVKKPGASVKVSCKASGFNIKDYYMHWVRQAPGQRLE |
| | | WMGWIDLENANTIYDAKFQGRVTITRDTSASTAYMELSSLRSEDTA |
| | | VYYCARDAYGRYFYDVWGQGTLVTVSSGGGGSGGGGGGGGSDIVM |
| | | TQSPDSLAVSLGERATINCKSSQSLLNARTGKNYLAWYQQKPGQPP |
| | | KLLIYWASTRESGVPDRFSGSGSGTDFTLTISSLQAEDVAVYYCKQ |
| | | SYSRRTFGGGTKVEIK |
| 44 | AC15J | QVQLVQSGAEVKKPGASVKVSCKASGFNIKDYYMHWVRQAPGQRLE |
| | | WMGWIDLENANTIYDAKFQGRVTITRDTSASTAYMELSSLRSEDTA |
| | | VYYCARDAYGRYFYDVWGQGTLVTVSSGGGGSGGGGSGGGGSDIVM |
| | | TQSPDSLAVSLGERATINCKSSQSLLNARTGKNYLAWYQQKPGQPP |
| | | KLLIYWASTRESGVPDRFSGSGSGTDFTLTISSLQAEDVAVYYCKQ |
| | | SYSRRTFGGGTKVEIKGGGGSGGGGSGGGGSQEDERIVLVDNKCKC |
| | | ARITSRIIRSSEDPNEDIVERNIRIIVPLNNRENISDPTSPLRTRF |
| | | VYHLSDLCKKCDPTEVELDNQIVTATQSNICDEDSATETCYTYDRN |
| | | KCYTAVVPLVYGGETKMVETALTPDACYPD |
| 45 | AC15J* | QVQLVQSGAEVKKPGASVKVSCKASGFNIKDYYMHWVRQAPGQRLE |
| | | WMGWIDLENANTIYDAKFQGRVTITRDTSASTAYMELSSLRSEDTA |
| | | VYYCARDAYGRYFYDVWGQGTLVTVSSGGGGGGGGSGGGGSDIVM |
| | | TQSPDSLAVSLGERATINCKSSQSLLNARTGKNYLAWYQQKPGQPP |
| | | KLLIYWASTRESGVPDRFSGSGSGTDFTLTISSLQAEDVAVYYCKQ |
| | | SYSRRTFGGGTKVEIKGGGGSGGGGSGGGGSQEDERIVLVDNKCKC |
| | | ARITSRIIRSSEDPNEDIVERNIRIIVPLNNRENISDPTSPLRTRF |
| | | VYHLSDLCKKCDPTEVELDNQIVTATQSNICDEDSATETCATYDRN |
| | | KCYTAVVPLVYGGETKMVETALTPDACYPD |
| 46 | AC15 J15HSA | QVQLVQSGAEVKKPGASVKVSCKASGFNIKDYYMHWVRQAPGQRLE |
| | | WMGWIDLENANTIYDAKFQGRVTITRDTSASTAYMELSSLRSEDTA |
| | | VYYCARDAYGRYFYDVWGQGTLVTVSSGGGGSGGGGGGGGSDIVM |
| | | TQSPDSLAVSLGERATINCKSSQSLLNARTGKNYLAWYQQKPGQPP |
| | | KLLIYWASTRESGVPDRFSGSGSGTDFTLTISSLQAEDVAVYYCKQ |
| | | SYSRRTFGGGTKVEIKGGGGSGGGGSGGGGSDAHKSEVAHRFKDLG |
| | | EENFKALVLIAFAQYLQQCPFEDHVKLVNEVTEFAKTCVADESAEN |
| | | CDKSLHTLFGDKLCTVATLRETYGEMADCCAKQEPERNECFLQHKD |
| | | DNPNLPRLVRPEVDVMCTAFHDNEETFLKKYLYEIARRHPYFYAPE |
| | | LLFFAKRYKAAFTECCQAADKAACLLPKLDELRDEGKASSAKQRLK |
| | | CASLQKFGERAFKAWAVARLSQRFPKAEFAEVSKLVTDLTKVHTEC |
| | | CHGDLLECADDRADLAKYICENQDSISSKLKECCEKPLLEKSHCIA |
| | | EVENDEMPADLPSLAADFVESKDVCKNYAEAKDVFLGMFLYEYARR |
| | | HPDYSVVLLLRLAKTYETTLEKCCAAADPHECYAKVFDEFKPLVEE |
| | | PQNLIKQNCELFKQLGEYKFQNALLVRYTKKVPQVSTPTLVEVSRN |
| | | LGKVGSKCCKHPEAKRMPCAEDYLSVVLNQLCVLHEKTPVSDRVTK |
| | | CCTESLVNRRPCFSALEVDETYVPKEFNAETFTFHADICTLSEKER |
| | | QIKKQTALVELVKHKPKATKEQLKAVMDDFAAFVEKCCKADDKETC |
| | | FAEEGKKLVAASQAALGL |
| 47 | AD scFv | QVQLVQSGAEVKKPGASVKVSCKASGFNIKDYYMHWVRQAPGQRLE |
| | | WIGWIDLENANTVYDAKFQGRVTITRDTSASTAYMELSSLRSEDTA |
| | | VYYCARDAYGRYFYDVWGQGTLVTVSSGGGGSGGGGSGGGGSDIVM |
| | | TQSPDSLAVSLGERATINCKSSQSLLNARTGKNYLAWYQQKPGQPP |
| | | KLLIYWASTRESGVPDRFSGSGSGTDFTLTISSLQAEDVAVYYCKQ |
| | | SYFRRTFGGGTKVEIK |
| 48 | AD15J | QVQLVQSGAEVKKPGASVKVSCKASGFNIKDYYMHWVRQAPGQRLE |
| | | WIGWIDLENANTVYDAKFQGRVTITRDTSASTAYMELSSLRSEDTA |
| | | VYYCARDAYGRYFYDVWGQGTLVTVSSGGGGSGGGGSGGGGSDIVM |
| | | TQSPDSLAVSLGERATINCKSSQSLLNARTGKNYLAWYQQKPGQPP |
| | | KLLIYWASTRESGVPDRFSGSGSGTDFTLTISSLQAEDVAVYYCKQ |
| | | SYFRRTFGGGTKVEIKGGGGSGGGGSGGGGSQEDERIVLVDNKCKC |
| | | ARITSRIIRSSEDPNEDIVERNIRIIVPLNNRENISDPTSPLRTRF |
| | | VYHLSDLCKKCDPTEVELDNQIVTATQSNICDEDSATETCYTYDRN |
| | | KCYTAVVPLVYGGETKMVETALTPDACYPD |
| 49 | AD15J* | QVQLVQSGAEVKKPGASVKVSCKASGFNIKDYYMHWVRQAPGQRLE |
| | | WIGWIDLENANTVYDAKFQGRVTITRDTSASTAYMELSSLRSEDTA |
| | | VYYCARDAYGRYFYDVWGQGTLVTVSSGGGGSGGGGSGGGGSDIVM |
| | | TQSPDSLAVSLGERATINCKSSQSLLNARTGKNYLAWYQQKPGQPP |
| | | KLLIYWASTRESGVPDRFSGSGSGTDFTLTISSLQAEDVAVYYCKQ |
| | | SYFRRTFGGGTKVEIKGGGGSGGGGSGGGGSQEDERIVLVDNKCKC |
| | | ARITSRIIRSSEDPNEDIVERNIRIIVPLNNRENISDPTSPLRTRF |
| | | VYHLSDLCKKCDPTEVELDNQIVTATQSNICDEDSATETCATYDRN |
| | | KCYTAVVPLVYGGETKMVETALTPDACYPD |
| 50 | AD15J15HSA | QVQLVQSGAEVKKPGASVKVSCKASGFNIKDYYMHWVRQAPGQRLE |
| | | WIGWIDLENANTVYDAKFQGRVTITRDTSASTAYMELSSLRSEDTA |
| | | VYYCARDAYGRYFYDVWGQGTLVTVSSGGGGSGGGGSGGGGSDIVM |
| | | TQSPDSLAVSLGERATINCKSSQSLLNARTGKNYLAWYQQKPGQPP |
| | | KLLIYWASTRESGVPDRFSGSGSGTDFTLTISSLQAEDVAVYYCKQ |
| | | SYFRRTFGGGTKVEIKGGGGSGGGGSGGGGSQEDERIVLVDNKCKC |
| | | ARITSRIIRSSEDPNEDIVERNIRIIVPLNNRENISDPTSPLRTRF |
| | | VYHLSDLCKKCDPTEVELDNQIVTATQSNICDEDSATETCYTYDRN |
| | | KCYTAVVPLVYGGETKMVETALTPDACYPDGGGGSGGGGSGGGGSD |
| | | AHKSEVAHRFKDLGEENFKALVLIAFAQYLQQCPFEDHVKLVNEVT |
| | | EFAKTCVADESAENCDKSLHTLFGDKLCTVATLRETYGEMADCCAK |
| | | QEPERNECFLQHKDDNPNLPRLVRPEVDVMCTAFHDNEETFLKKYL |
| | | YEIARRHPYFYAPELLFFAKRYKAAFTECCQAADKAACLLPKLDEL |
| | | RDEGKASSAKQRLKCASLQKFGERAFKAWAVARLSQRFPKAEFAEV |
| | | SKLVTDLTKVHTECCHGDLLECADDRADLAKYICENQDSISSKLKE |
| | | CCEKPLLEKSHCIAEVENDEMPADLPSLAADFVESKDVCKNYAEAK |
| | | DVFLGMFLYEYARRHPDYSVVLLLRLAKTYETTLEKCCAAADPHEC |
| | | YAKVFDEFKPLVEEPQNLIKQNCELFKQLGEYKFQNALLVRYTKKV |
| | | PQVSTPTLVEVSRNLGKVGSKCCKHPEAKRMPCAEDYLSVVLNQLC |
| | | VLHEKTPVSDRVTKCCTESLVNRRPCFSALEVDETYVPKEFNAETF |
| | | TFHADICTLSEKERQIKKQTALVELVKHKPKATKEQLKAVMDDFAA |
| | | FVEKCCKADDKETCFAEEGKKLVAASQAALGL |
| 51 | AE scFv | QVQLVQSGAEVKKPGASVKVSCKASGFNIKDYYMHWVRQAPGQRLE |
| | | WIGWIDLENANTVYDAKFQGRVTITRDTSASTAYMELSSLRSEDTA |
| | | VYYCARDAYGQYFYDVWGQGTLVTVSSGGGGGGGGSGGGGSDIVM |
| | | TQSPDSLAVSLGERATINCKSSQSLLNARTGKNYLAWYQQKPGQPP |
| | | KLLIYWASTRESGVPDRFSGSGSGTDFTLTISSLQAEDVAVYYCTQ |
| | | SYFRRTFGGGTKVEIK |
| 52 | AE15J | QVQLVQSGAEVKKPGASVKVSCKASGFNIKDYYMHWVRQAPGQRLE |
| | | WIGWIDLENANTVYDAKFQGRVTITRDTSASTAYMELSSLRSEDTA |
| | | VYYCARDAYGQYFYDVWGQGTLVTVSSGGGGSGGGGSGGGGSDIVM |
| | | TQSPDSLAVSLGERATINCKSSQSLLNARTGKNYLAWYQQKPGQPP |
| | | KLLIYWASTRESGVPDRFSGSGSGTDFTLTISSLQAEDVAVYYCTQ |
| | | SYFRRTFGGGTKVEIKGGGGSGGGGSGGGGSQEDERIVLVDNKCKC |
| | | ARITSRIIRSSEDPNEDIVERNIRIIVPLNNRENISDPTSPLRTRF |
| | | VYHLSDLCKKCDPTEVELDNQIVTATQSNICDEDSATETCYTYDRN |
| | | KCYTAVVPLVYGGETKMVETALTPDACYPD |
| 53 | AE15J* | QVQLVQSGAEVKKPGASVKVSCKASGFNIKDYYMHWVRQAPGQRLE |
| | | WIGWIDLENANTVYDAKFQGRVTITRDTSASTAYMELSSLRSEDTA |
| | | VYYCARDAYGQYFYDVWGQGTLVTVSSGGGGSGGGGSGGGGSDIVM |
| | | TQSPDSLAVSLGERATINCKSSQSLLNARTGKNYLAWYQQKPGQPP |
| | | KLLIYWASTRESGVPDRFSGSGSGTDFTLTISSLQAEDVAVYYCTQ |
| | | SYFRRTFGGGTKVEIKGGGGSGGGGSGGGGSQEDERIVLVDNKCKC |
| | | ARITSRIIRSSEDPNEDIVERNIRIIVPLNNRENISDPTSPLRTRF |
| | | VYHLSDLCKKCDPTEVELDNQIVTATQSNICDEDSATETCATYDRN |
| | | KCYTAVVPLVYGGETKMVETALTPDACYPD |
| 54 | AE15J15HSA | QVQLVQSGAEVKKPGASVKVSCKASGFNIKDYYMHWVRQAPGQRLE |
| | | WIGWIDLENANTVYDAKFQGRVTITRDTSASTAYMELSSLRSEDTA |
| | | VYYCARDAYGQYFYDVWGQGTLVTVSSGGGGSGGGGGGGGSDIVM |
| | | TQSPDSLAVSLGERATINCKSSQSLLNARTGKNYLAWYQQKPGQPP |
| | | KLLIYWASTRESGVPDRFSGSGSGTDFTLTISSLQAEDVAVYYCTQ |
| | | SYFRRTFGGGTKVEIKGGGGSGGGGSGGGGSQEDERIVLVDNKCKC |
| | | ARITSRIIRSSEDPNEDIVERNIRIIVPLNNRENISDPTSPLRTRF |
| | | VYHLSDLCKKCDPTEVELDNQIVTATQSNICDEDSATETCYTYDRN |
| | | KCYTAVVPLVYGGETKMVETALTPDACYPDGGGGSGGGGSGGGGSD |
| | | AHKSEVAHRFKDLGEENFKALVLIAFAQYLQQCPFEDHVKLVNEVT |
| | | EFAKTCVADESAENCDKSLHTLFGDKLCTVATLRETYGEMADCCAK |
| | | QEPERNECFLQHKDDNPNLPRLVRPEVDVMCTAFHDNEETFLKKYL |
| | | YEIARRHPYFYAPELLFFAKRYKAAFTECCQAADKAACLLPKLDEL |
| | | RDEGKASSAKQRLKCASLQKFGERAFKAWAVARLSQRFPKAEFAEV |
| | | SKLVTDLTKVHTECCHGDLLECADDRADLAKYICENQDSISSKLKE |
| | | CCEKPLLEKSHCIAEVENDEMPADLPSLAADFVESKDVCKNYAEAK |
| | | DVFLGMFLYEYARRHPDYSVVLLLRLAKTYETTLEKCCAAADPHEC |
| | | YAKVFDEFKPLVEEPQNLIKQNCELFKQLGEYKFQNALLVRYTKKV |
| | | PQVSTPTLVEVSRNLGKVGSKCCKHPEAKRMPCAEDYLSVVLNQLC |
| | | VLHEKTPVSDRVTKCCTESLVNRRPCFSALEVDETYVPKEFNAETF |
| | | TFHADICTLSEKERQIKKQTALVELVKHKPKATKEQLKAVMDDFAA |
| | | FVEKCCKADDKETCFAEEGKKLVAASQAALGL |
| 55 | 1.5.3 HCDR1 | GYSFTSYWIG |
| | US10, 787,520 | |
| 56 | 1.5.3 HCDR2 | IIYPGDSDTRYSPSFQG |
| | US10,787, 520 | |
| 57 | 1.5.3 HCDR3 | HPSYGSGSPNFDY |
| | US10, 787,520 | |
| 58 | 1.5.3 LCDR1 | RSSQSLVYSDGNTYLS |
| | US10, 787,520 | |
| 59 | 1.5.3 LCDR2 | KISNRFS |
| | US10, 787, 520 | |
| 60 | 1.5.3 LCDR3 | VQATQFPLT |
| | US10, 787, 520 | |
| 61 | IGM-2323 Heavy | EVQLVQSGAEVKKPGESLKISCKGSGYSFTSYWIGWVRQMPGKGLE |
| | Chain | WMGIIYPGDSDTRYSPSFQGQVTISADKSITTAYLQWSSLKASDTA |
| | | MYYCARHPSYGSGSPNFDYWGQGTLVTVSSGSASAPTLFPLVSCEN |
| | | SPSDTSSVAVGCLAQDFLPDSITFSWKYKNNSDISSTRGFPSVLRG |
| | | GKYAATSQVLLPSKDVMQGTDEHVVCKVQHPNGNKEKNVPLPVIAE |
| | | LPPKVSVFVPPRDGFFGNPRKSKLICQATGFSPRQIQVSWLREGKQ |
| | | VGSGVTTDQVQAEAKESGPTTYKVTSTLTIKESDWLSQSMFTCRVD |
| | | HRGLTFQQNASSMCVPDQDTAIRVFAIPPSFASIFLTKSTKLTCLV |
| | | TDLTTYDSVTISWTRQNGEAVKTHTNISESHPNATFSAVGEASICE |
| | | DDWNSGERFTCTVTHTDLPSPLKQTISRPKGVALHRPDVYLLPPAR |
| | | EQLNLRESATITCLVTGFSPADVFVQWMQRGQPLSPEKYVTSAPMP |
| | | EPQAPGRYFAHSILTVSEEEWNTGETYTCVVAHEALPNRVTERTVD |
| | | KSTGKPTLYNVSLVMSDTAGTCY |
| 62 | IGM-2323 Light | DIVMTQTPLSSPVTLGQPASISCRSSQSLVYSDGNTYLSWLQQRPG |
| | Chain | QPPRLLIYKISNRFSGVPDRFSGSGAGTDFTLKISRVEAEDVGVYY |
| | | CVQATQFPLTFGGGTKVEIKRTVAAPSVFIFPPSDEQLKSGTASVV |
| | | CLLNNFYPREAKVQWKVDNALQSGNSQESVTEQDSKDSTYSLSSTL |
| | | TLSKADYEKHKVYACEVTHQGLSSPVTKSFNRGEC |
| 63 | CD20xCD3 IgG Heavy | EVQLVESGGGLVQPGRSLRLSCVASGFTENDYAMHWVRQAPGKGLE |
| | Chain #1 | WVSVISWNSDSIGYADSVKGRFTISRDNAKNSLYLQMHSLRAEDTA |
| | | LYYCAKDNHYGSGSYYYYQYGMDVWGQGTTVTVSSASTKGPSVFPL |
| | | APCSRSTSESTAALGCLVKDYFPEPVTVSWNSGALTSGVHTFPAVL |
| | | QSSGLYSLSSVVTVPSSSLGTKTYTCNVDHKPSNTKVDKRVESKYG |
| | | PPCPPCPAPPVAGPSVFLFPPKPKDTLMISRTPEVTCVVVDVSQED |
| | | PEVQFNWYVDGVEVHNAKTKPREEQFNSTYRVVSVLTVLHQDWING |
| | | KEYKCKVSNKGLPSSIEKTISKAKGQPREPQVYTLPPSQEEMTKNQ |
| | | VSLTCLVKGFYPSDIAVEWESNGQPENNYKTTPPVLDSDGSFFLYS |
| | | RLTVDKSRWQEGNVFSCSVMHEALHNHYTQKSLSLSLG |
| 64 | CD20xCD3 IgG Light | EIVMTQSPATLSVSPGERATLSCRASQSVSSNLAWYQQKPGQAPRL |
| | Chain | LIYGASTRATGIPARFSGSGSGTEFTLTISSLQSEDFAVYYCQHYI |
| | | NWPLTFGGGTKVEIKRTVAAPSVFIFPPSDEQLKSGTASVVCLLNN |
| | | FYPREAKVQWKVDNALQSGNSQESVTEQDSKDSTYSLSSTLTLSKA |
| | | DYEKHKVYACEVTHQGLSSPVTKSENRGEC |
| 65 | CD20xCD3 IgG Heavy | EVQLVESGGGLVQPGRSLRLSCAASGFTFDDYTMHWVRQAPGKGLE |
| | Chain #2 | WVSGISWNSGSIGYADSVKGRFTISRDNAKKSLYLQMNSLRAEDTA |
| | | LYYCAKDNSGYGHYYYGMDVWGQGTTVTVASASTKGPSVFPLAPCS |
| | | RSTSESTAALGCLVKDYFPEPVTVSWNSGALTSGVHTFPAVLQSSG |
| | | LYSLSSVVTVPSSSLGTKTYTCNVDHKPSNTKVDKRVESKYGPPCP |
| | | PCPAPPVAGPSVFLFPPKPKDTLMISRTPEVTCVVVDVSQEDPEVQ |
| | | FNWYVDGVEVHNAKTKPREEQFNSTYRVVSVLTVLHQDWLNGKEYK |
| | | CKVSNKGLPSSIEKTISKAKGQPREPQVYTLPPSQEEMTKNQVSLT |
| | | CLVKGFYPSDIAVEWESNGQPENNYKTTPPVLDSDGSFFLYSRLTV |
| | | DKSRWQEGNVFSCSVMHEALHNRFTQKSLSLSLG |

1. A method of treating cancer in a subject in need thereof, comprising administering a dosage of 75 mg to 600 mg of a T cell engaging molecule to the subject,
wherein the T cell engaging molecule is a multimeric binding molecule comprising five bivalent binding units and a modified J-chain, wherein each binding unit comprises two IgM heavy chains, each comprising a heavy chain variable region (VH) and an IgM constant region and two light chains, each comprising a light chain variable region (VL) and a light chain constant region,
wherein an associated VH and VL specifically bind to CD20 or a subunit thereof,
wherein the VH comprises three immunoglobulin complementarity determining regions: HCDR1, HCDR2, and HCDR3, and the VL comprises three immunoglobulin complementarity determining regions: LCDR1, LCDR2, and LCDR3, wherein the HCDR1, HCDR2, HCDR3, LCDR1, LCDR2, and LCDR3 comprise, respectively, the amino acid sequence of SEQ ID NO: 55; SEQ ID NO: 56; SEQ ID NO: 57; SEQ ID NO: 58; SEQ ID NO: 59; and SEQ ID NO: 60,
wherein the modified J-chain comprises a J-chain or functional fragment or variant thereof and an scFv molecule that specifically binds to CD3,
and wherein the scFv comprises the amino acid sequence of SEQ ID NO: 31.

2. The method of claim 1, wherein the dosage is 100 mg to 300 mg.

3. The method of claim 2, wherein the subject had received a prior dosage of the T cell engaging molecule, wherein the prior dosage was less than 100 mg.

4. The method of claim 3, wherein the prior dosage comprised two or three prior dosages of the T cell engaging molecule, wherein the two or three prior dosages were less than 100 mg.

5. The method of claim 1, further comprising:
(a) administering a dosage of 10 mg to 50 mg of the T cell engaging molecule to the subject; and
(b) administering the dosage of 75 mg to 600 mg of the T cell engaging molecule to the subject at least 5 days after the administration of step (a).

6. The method of claim 5, further comprising (c) administering the dosage of the T cell engaging molecule administered to the subject in step (b) at least days after the administration of step (b).

7. The method of claim 5, wherein the dosage of the T cell engaging molecule in step (b) is 100 mg to 300 mg, 100 mg, or 300 mg.

8. A method of treating cancer in a subject in need thereof, comprising:
(a) administering to the subject a T cell engaging molecule at a determined dosage;
(b) monitoring the subject for an administration-related symptom; and
(c)(i) administering the T cell engaging molecule to the subject at the same or a reduced dosage relative to the determined dosage if the subject had the administration-related symptom, and
(c)(ii) administering the T cell engaging molecule to the subject at an increased dosage relative to the determined dosage if the subject did not have the administration-related symptom,
wherein the T cell engaging molecule is a multimeric binding molecule comprising five bivalent binding units and a modified J-chain, wherein each binding unit comprises two IgM heavy chains, each comprising a heavy chain variable region (VH) and an IgM constant region and two light chains, each comprising a light chain variable region (VL) and a light chain constant region,
wherein an associated VH and VL specifically bind to CD20 or a subunit thereof,
wherein the VH comprises three immunoglobulin complementarity determining regions: HCDR1, HCDR2, and HCDR3, and the VL comprises three immunoglobulin complementarity determining regions: LCDR1, LCDR2, and LCDR3, wherein the HCDR1, HCDR2, HCDR3, LCDR1, LCDR2, and LCDR3 comprise, respectively, the amino acid sequence of SEQ ID NO: 55; SEQ ID NO: 56; SEQ ID NO: 57; SEQ ID NO: 58; SEQ ID NO: 59; and SEQ ID NO: 60,
wherein the modified J-chain comprises a J-chain or functional fragment or variant thereof and an scFv molecule that specifically binds to CD3,
wherein the scFv comprises the amino acid sequence of SEQ ID NO: 31.

9. The method of claim 8, wherein the administration-related symptom comprises hypotension, chills, fever, elevated C reactive protein (CRP) level, or a combination thereof.

10. The method of claim 8, further comprising:
(d) repeating steps (b) and (c) one or more times.

11. The method of claim 8, wherein the increased dosage of the T cell engaging molecule in step (c)(ii) is:
(a) 25%-1000% greater than the determined dosage of the T cell engaging molecule;
(b) 30 mg to 300 mg;
(c) 100 mg to 300 mg; or
(d) 100 mg or 300 mg.

12. The method of claim 1, wherein the subject had previously received a cancer therapy, wherein the cancer therapy comprises chemotherapy, radiation therapy, or immunotherapy, wherein the immunotherapy is different from the T cell engaging molecule.

13. The method of claim 12, wherein the immunotherapy is rituximab.

14-21. (canceled)

22. The method of claim 1, wherein the J-chain or functional fragment or variant thereof comprises SEQ ID NO: 7.

23. (canceled)

24. The method of claim 1, wherein the J-chain or fragment or variant thereof comprises SEQ ID NO: 34.

25. The method of claim 1, wherein the heavy chain comprises SEQ ID NO: 61 and the light chain comprises SEQ ID NO: 62.

26. The method of claim 1, wherein the subject is human.

27. The method of claim 26, wherein the cancer is a CD20 positive cancer and/or the cancer is relapsed or refractory cancer.

28. The method of claim 26, wherein the cancer is a leukemia, lymphoma, or myeloma.

29. The method of claim 28, wherein the cancer is non-Hodgkin lymphoma (NHL).

30. (canceled)
